(19) United States
(12) Patent Application Publication
(10) Pub. No.: US 2005/0065118 A1
(43) Pub. Date: Mar. 24, 2005

(54) Organosulfur inhibitors of tyrosine phosphatases

(76) Inventors: Jing Wang, San Diego, CA (US); Darryl Rideout, San Diego, CA (US); Kalyanaraman Ramnarayan, San Diego, CA (US); Chung-Ying Tsai, San Diego, CA (US); Venkatachalapathi V. Yalamoori, San Diego, CA (US); Feiyue Wu, Poway, CA (US); Colin Loweth, San Diego, CA (US); Hassan ElAbdellaoui, San Diego, CA (US); Leah Fung, San Diego, CA (US); Thomas P. Brady, San Diego, CA (US)

Correspondence Address: James C Weseman;The Law Offices of James C Weseman, Suite 1600, 401 West A Street, San Diego, CA, 92101, US

(21) Appl. No.: 10/493,113

(22) PCT Filed: Oct. 16, 2002
(86) PCT No.: PCT/US02/33076

(30) Foreign Application Priority Data
Oct. 16, 2001 (US) 60329957

(51) Int. Cl. A61K 31/66; A61K 31/325

(52) U.S. Cl. 514/114; 514/483; 514/479

(57) ABSTRACT

Organosulfur modulators of tyrosine phosphatases and their use in the treatment of disease are disclosed.

### TECHNICAL FIELD

The present invention is directed to inhibiting the activity of tyrosine phosphatases that regulate signal transduction, and, more particularly, to the use of organosulfur compositions as tyrosine phosphatase inhibitors for the treatment of diseases which respond to phosphatase inhibition.

### BACKGROUND OF THE INVENTION

Cellular signal transduction is a fundamental mechanism whereby external stimuli that regulate cellular processes are relayed to the interior of cells. The biochemical pathways through which signals are transmitted within cells comprise a circuitry of directly or functionally connected interactive proteins. One of the key biochemical mechanisms of signal transduction involves the reversible phosphorylation of tyrosine residues on proteins. The phosphorylation state of a protein may affect its conformation and/or enzymatic activity as well as its cellular location. The phosphorylation state of a protein is modified through the reciprocal actions of protein tyrosine kinases (PTKs) and protein tyrosine phosphatases (TPs) at various specific tyrosine residues.

A common mechanism by which receptors regulate cell function is through an inducible tyrosine kinase activity which is either endogenous to the receptor or is imparted by other proteins that become associated with the receptor (Damell et al., 1994, Science 264:1415-1421; Heldin, 1995, Cell 80:213-223; Pawson, 1995, Nature 373:573-580).

Protein tyrosine kinases comprise a large family of transmembrane receptor and intracellular enzymes with multiple functional domains (Taylor et al., 1992 Ann. Rev. Cell Biol. 8:429-62). The binding of ligand allosterically transduces a signal across the cell membrane where the cytoplasmic portion of the PTKs initiates a cascade of molecular interactions that disseminate the signal throughout the cell and into the nucleus. Many receptor protein tyrosine kinase (RPTKs), such as epidermal growth factor receptor (EGFR) and platelet-derived growth factor receptor (PDGFR) undergo oligomerization upon ligand binding, and the receptors self-phosphorylate (via autophosphorylation or transphosphorylation) on specific tyrosine residues in the cytoplasmic portions of the receptor (Schlessinger and Ullrich, 1992, Neuron, 9:383-91, Heldin, 1995, Cell 80:213-223). Cytoplasmic protein tyrosine kinases (CPTKs), such as Janus kinases (e.g., JAK1, JAK2, TYK2) and Src kinases (e.g., src, ick, fyn), are associated with receptors for cytokines (e.g., IL-2, IL-3, IL-6, erythropoietin) and interferons, and antigen receptors. These receptors also undergo oligomerization and have tyrosine residues that become phosphorylated during activation, but the receptor polypeptides themselves do not possess kinase activity.

Like the PTKs, the protein tyrosine phosphatases (PTJs) comprise a family of transmembrane and cytoplasmic enzymes, possessing at least an approximately 230 amino acid catalytic domain containing a highly conserved active site with the consensus motif >I/VIHCXAGXXR>S/TIG. The substrates of PTPs may be PTKs which possess phosphotyrosine residues or the substrates of PTKs (Hunter, 1989, Cell 58:1013-16; Fischer et al., 1991, Science 253:401-6; Saito & Streuli, 1991, Cell Growth and Differentiation 2:59-65; Pot and Dixon, 1992, Biochemn. Biophys. Acta 1136:35-43).

Transmembrane or receptor-like PTPs (RPTPs) possess an extracellular domain, a single transmembrane domain, and one or two catalytic domains followed by a short cytoplasmic tail. The extracellular domains of these RPTPs are highly divergent, with small glycosylated segments (e.g., RPTPα, RPTPε), tandem repeats of immunoglobulin-like and/or fibronectin type Im domains (e.g., LAR) or carbonic anhydrase like domains (e.g., RPTPγ, RPTPβ). These extracellular features might suggest that these RPTPs function as a receptor on the cell surface, and their enzymatic activity might be modulated by ligands. Intracellular or cytoplasmic PTPs (CPTPs), such as PTP1C, PTP1D, typically contain a single catalytic domain flanked by several types of modular conserved domains. For example, PTP1C, a hemopoietic cell CPTP is characterized by two Src-homology homology 2 (SH2) domains that recognize short peptide motifs bearing phosphotyrosine (pTyr).

In general, these modular conserved domains influence the intracellular localization of the protein. SH2-containing proteins are able to bind pTyr sites in activated receptors and cytoplasmic phosphoproteins. Another conserved domain known as SH3 binds to proteins with proline-rich regions. A third type known as pleckstrin-homology (PH) domain has also been identified. These modular domains have been found in both CPTKs and CPTPs as well as in non-catalytic adapter molecules, such as Grbs (Growth factor Receptor Bound), which mediate protein-protein interactions between components of the signal transduction pathway (Skoliik et al., 1991, Cell 65:83-90; Pawson, 1995, Nature 373:573-580).

Multiprotein signaling complexes comprising receptor subunits, kinases, phosphatases and adapter molecules are assembled in subcellular compartments through the specific and dynamic interactions between these domains with their binding motifs. Such signaling complexes integrate the extracellular signal from the ligand-bound receptor and relay the signal to other downstream signaling proteins or complexes in other locations inside the cell or in the nucleus (Koch et al., 1991, Science 252:668-674; Pawson, 1994, Nature 373:573-580; Mauro et al., 1994, Trends Biochem Sci 19:151-155; Cohen et al., 1995, Cell 80:237-248).

The levels of tyrosine phosphorylation required for normal cell growth and differentiation at any time are achieved through the coordinated action of PTKs and PTPS. Depending on the cellular context, these two types of enzymes may either antagonize or cooperate with each other during signal transduction. An imbalance between these enzymes may impair normal cell functions leading to metabolic disorders and cellular transformation.

For example, insulin binding to the insulin receptor, which is a PTK, triggers a variety of metabolic and growth promoting effects such as glucose transport, biosynthesis of glycogen and fats, DNA synthesis, cell division and differentiation. Diabetes mellitus, which is characterized by insufficient or a lack of insulin signal transduction, can be caused by any abnormality at any step along the insulin signaling pathway (Olefsky, 1988, in “Cecil Textbook of Medicine,” 18th Ed., 2:1360-81).

It is also well known, for example, that the overexpression of PTKs, such as HER2, can play a decisive role in the development of cancer (Slamon et al., 1987, Science 235:77-82) and that antibodies capable of blocking the activity of this enzyme can abrogate tumor growth (Drebin et al., 1988, Oncogenze 2:387-394). Blocking the signal transduction capability of tyrosine kinases such as Flk-1 and the PDGF receptor have been shown to block tumor growth in animal models (Millauer et al., 1994, Nature 367:577; Ueno et al., Science 252:844-848).

Relatively less is known with respect to the direct role of tyrosine phosphatases in signal transduction; PTPs may play a role in human diseases. For example, ectopic expression of RPTPa produces a transformed phenotype in embryonic fibroblasts (Zheng et al., Nature 359:336-339), and overexpression of RPTPA in embryonal carcinoma cells causes the cells to differentiate into a cell type with neuronal phenotype (den Hertog et al., EMBO J. 12:3789-3798). The gene for human RPTPγ has been localized to chromosome 3p21 which is a segment frequently altered in renal and small lung carcinoma. Mutations may occur in the extracellular segment of RPTPγ which result in RPTPs that no longer respond to external signals (LaForgia et al., Wary et al., 1993, Cancer Res 52:478-482). Mutations in the gene encoding PTP1C (also known as HCP, SHP) are the cause of the motheaten phenotype in mice which suffer severe immunodeficiency, and systemic autoinmmune disease accompanied by hyperproliferation of macrophages (Schultz et al., 1993, Cell 73:1445-1454). PTP1D (also known as Syp or PTP2C) has been shown to bind through SH2 domains to sites of phosphorylation in PDGFR, EGFR and insulin receptor substrate 1 (IRS-1). Reducing the activity of PTP1D by microinjection of anti-PTP1D antibody has been shown to block insulin or EGF-induced mitogenesis (ciao et al., 1994, J Biol Chem 269:21244-21248).

### DISCLOSURE OF THE INVENTION

The present invention provides methods and compositions for the modulation of tyrosine phosphatase activity. Such compositions and methods will find use in the treatment of diseases caused by dysfunctional signal transduction.

In one aspect the present invention provides a method for inhibiting protein tyrosine phosphatase activity which comprises administering to a mammal an effective amount of a compound having the formula: 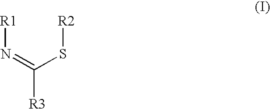
or a pharmaceutically-acceptable salt thereof, wherein:
R1, R2, and R3 are each independently selected from
   H, hydroxyl, alkoxy, alkylthio, nitro, amino or amido (each optionally substituted with alkyl, amino, cycloheteroalkyl, cycloalkyl fluoro, aryl, heteroaryl, cycloheteroalkyl, alkylthio, arylthio, cyano, OR′, OC═OR″, C═O—OR′″, or C═O—NR″″R″″);
   small alkyl (C1-C10) (optionally C1-C6) (optionally substituted with alkyl, amino, cycloheteroalkyl, cycloalkyl fluoro, aryl, heteroaryl, cycloheteroalkyl, alkylthio, P═O(OR″)₂, CH₂P═O(OR″)₂, CF₂P═O(OR″)₂, NHCOCOOR″, CH(COOR″)₂, arylthio, cyano, OR″, OC═OR″, C═O—OR′″, or C═O—NR″″R″″);
   phenyl and mono and disubstituted (at positions 3 and 4) phenyl (wherein the phenyl ring is independently substituted with alkyl, trifluoromethyl, mono and di halogen atoms, alkylthio, alkoxy, nitro, cyano, morphilino, cyclohexyl, phenyl, phenolic, dioxymethylene, nitro, acetylamino, OR′, P═O(OR″)₂, CH₂P═O(OR″)₂, CF₂P═O(OR″)₂, NHCOCOOR″, CH(COOR″)₂);
   heteroaryl, cycloheteroalkyl and cycloheteroalkyl (each optionally substituted with alkyl, halogen, alkylthio, alkoxy, or nitro, OR′, P═O(OR″)₂, CH₂P═O(OR″)₂, CF₂P═O(OR″)₂, NHCOCOOR″, CH(COOR″)_{2,});
   cycloalkyl (C3-C10) (optionally substituted with alkyl, fluoro, aryl, heteroaryl, cycloheteroalkyl, cycloheteroalkyl alkylthio, arylthio, cyano, P═O(OR″)₂, CH₂P═O(OR″)₂, CF₂P═O(OR″)₂, NHCOCOOR″, CH(COOR″)₂, OR′, OC═OR′, C═O—OR″, or C═O—NR′″R″″);
   alkenyl (C1-C10) (optionally substituted with alkyl, fluoro, aryl, heteroaryl, cycloheteroalkyl, cycloheteroalkyl, alkylthio, arylthio, cyano, P═O(OR′″)₂, CH₂P═O(OR″)₂, CF₂P═O(OR″)₂, NHCOCOOR″, CH(COOR″)₂, OR′, OC═OR′, C═O—OR″, or C═O—NR′″R″″);
   alkadienyl (C1-C10) (optionally substituted with alkyl, fluoro, aryl, heteroaryl, cycloheteroalkyl, cycloheteroalkyl, alkylthio, arylthio, cyano, P═O(OR″)₂, CH₂P═O(OR″)₂, CF₂P═O(OR″)₂, NHCOCOOR″, CH(COOR″)₂, OR′, —OC═OR5, —C═O—OR″, C═O—NR′″R″″);
   cycloalkenyl (C4-C10), optionally substituted with alkyl, fluoro, aryl, heteroaryl, cycloheteroalkyl, cycloheteroalkyl, alkylthio, arylthio, cyano, P═O(OR″)₂, CH₂P═O(OR″)₂, CF₂P═O(OR″)₂, NHCOCOOR″, CH(COOR″)₂, OR′, —OC═OR′, —C═O—OR″, C═O—NR′″R″″;
   bicycloalkyl (C5-C12), optionally substituted with alkyl, fluoro, aryl, heteroaryl, cycloheteroalkyl, cycloheteroalkyl, alkylthio, arylthio, cyano, P═O(OR″)₂, CH₂P═O(OR″)₂, CF₂P═O(OR″)₂, NHCOCOOR″, CH(COOR″)₂, OR′, —OC═OR′, —C═O—OR″, C═O—NR′″R″″;
   tricycloalkyl (C8-C14), optionally substituted with alkyl, fluoro, aryl, heteroaryl, cycloheteroalkyl, cycloheteroalkyl, alkylthio, arylthio, cyano, P═O(OR″)₂, CH₂P═O(OR″)₂, CF₂P═O(OR″)₂, NHCOCOOR″, CH(COOR″)₂, OR′, —OC═OR′, —C═O—OR″, C—O—NR′″R″″;
      where
   Each R′ is independently:
      hydrogen;
      alkyl (C1-C10), optionally substituted with alkyl, keto, fluoro, alkoxy, alkylthio, aryl, heteroaryl, cycloheteroalkyl, cycloheteroalkyl, cyano, aryloxy, cycloalkyl, COOR″, P═O(OR″)₂, CH₂P═O(OR″)₂, CF₂P═O(OR′)₂, NHCOCOOR″, CH(COOR″)₂;
      aryl, optionally substituted with alkyl, keto, fluoro, alkoxy, alkylthio, cyano, aryloxy, COOR″, P═O(OR″)₂, CH₂P═O(OR″)₂, CF₂P═O(OR″)₂, NHCOCOOR″, CH(COOR″)₂;
      heteroaryl, cycloheteroalkyl, cycloheteroalkyl, optionally substituted with alkyl, keto, fluoro, alkoxy, alkylthio, cyano, aryloxy, COOR″, P═O(OR″)₂, CH₂P═O(OR″)₂, CF₂P═O(OR″)₂, NHCOCOOR″, CH(COOR″)₂;
      cycloalkyl, optionally substituted with alkyl, keto, fluoro, alkoxy, alkylthio, aryl, heteroaryl, cycloheteroalkyl, cycloheteroalkyl, cyano, COOR″, P═O(OR″)₂, CH₂P═O(OR″)₂, CF₂P═O(OR″)₂, NHCOCOOR″, CH(COOR″)₂;
   Each R″ is independently
      hydrogen,
      alkyl (C1-C10), optionally substituted with alkyl, keto, fluoro, alkoxy, alkylthio, aryl, heteroaryl, cycloheteroalkyl, cycloheteroalkyl;
      aryl, optionally substituted with alkyl, keto, fluoro, alkoxy, alkylthio;
      heteroaryl, cycloheteroallyl, optionally substituted with alkyl, keto, fluoro, alkoxy, alkylthio;
      cycloalkyl, optionally substituted with alkyl, keto, fluoro, alkoxy, alkylthio, aryl, heteroaryl, cycloheteroalkyl;
   Each R′″ is independently
      alkyl (C1-C10), optionally substituted with alkyl, keto, fluoro, alkoxy, alkylthio, aryl, heteroaryl, cycloheteroalkyl, COOR″, P═O(OR″)₂, CH₂P—O(OR″)₂, CF₂P═O(OR″)₂, NHCOCOOR″, CH(COOR″)₂;
      aryl, optionally substituted with alkyl, keto, fluoro, alkoxy, alkylthio;
      heteroaryl, cycloheteroalkyl, optionally substituted with alkyl, keto, fluoro, alkoxy, alkylthio, COOR″, P═O(OR″)₂, CH₂P═O(OR″)₂, CF₂P═O(OR′″)₂, NHCOCOOR″, CH(COOR″)₂;
      cycloalkyl, optionally substituted with alkyl, keto, fluoro, alkoxy, alkylthio, aryl, heteroaryl, cycloheteroalkyl, COOR″, P═O(OR″)₂, CH₂P═O(OR″)₂, CF₂P═O(OR″)₂, NHCOCOOR″, CH(COOR″)₂; and
   Each R″″ is independently
      alkyl (C1-C10), optionally substituted with alkyl, keto, fluoro, alkoxy, alkylthio, aryl, heteroaryl, cycloheteroalkyl, COOR″, P—O(OR″)₂, CH₂P═O(OR″)₂, CF₂P═O(OR″)₂, NHCOCOOR″, CH(COOR″)₂;
      aryl, optionally substituted with alkyl, keto, fluoro, alkoxy, alkylthio, COOR″, P═O(OR″)₂, CH₂P═O(OR″)₂, CF₂P═O(OR′″)₂, NHCOCOOR″, CH(COOR″)₂;
      heteroaryl, cycloheteroalkyl, optionally substituted with alkyl, keto, fluoro, alkoxy, alkylthio, COOR″, P═O(OR″)₂, CH₂P═O(OR″)₂, CF₂P═O(OR″)₂, NHCOCOOR″, CH(COOR″)₂; and
      cycloalkyl, optionally substituted with alkyl, keto, fluoro, alkoxy, alkylthio, aryl, heteroaryl, cycloheteroalky, COOR″, P═O(OR″)₂, CH₂P═O(OR″)₂, CF₂P═O(OR″)₂, NHCOCOOR″, CH(COOR″)₂.
         And wherein each of R1, R2 and R3 are linked to their respective core atoms through C, N, O or S of the substiuent group, provided that if R2 is to be linked through O or S, then the core atom S is oxidized.

### BRIEF DESCRIPTION OF THE DRAWINGS

FIG. 1 depicts selected compounds of the invention, together with chemical names.

### DETAILED DESCRIPTION OF THE INVENTION

The present invention provides methods and compositions for the inhibition of tyrosine phosphatase activity. Such compositions and methods will find use in the treatment of diseases caused by dysfunctional signal transduction.

In one aspect the present invention provides a method for inhibiting protein tyrosine phosphatase activity which comprises administering to a mammal an effective amount of a compound having the formula: 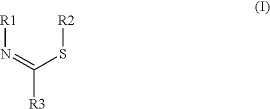
wherein R1, R2 and R3 are as further defined below, together with a pharmaceutically acceptable salt thereof.

The compounds of the present invention inhibit tyrosine phosphatases, including PTP-1B, and thus improve insulin sensitivity, among other benefits. The compounds therefore will find use in preventing or treating Type 1 and Type 2 diabetes [and associated complications such as hypertension, ischemic diseases of the large and small blood vessels, blindness, circulatory problems, kidney failure and atherosclerosis], syndrome X, metabolic syndrome, improving glucose tolerance, improving insulin sensitivity when there is insulin resistance, improving leptin sensitivity where there is leptin resistance, lowering body weight, and preventing or treating obesity. In addition, the compounds will be useful in preventing or treating cancer, neurodegenerative diseases, and the like.

The compounds of the present invention are generally characterized as nitrogen-containing organosulfur compounds having the formula (I) and their pharmaceutically acceptable salts: 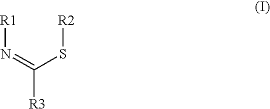
wherein:
R1, R2, and R3 are each independently selected from
   H, hydroxyl, alkoxy, alkylthio, nitro, amino or amido (each optionally substituted with alkyl, amino, cycloheteroalkyl, cycloalkyl fluoro, aryl, heteroaryl, cycloheteroalkyl, alkylthio, arylthio, cyano, OR′, OC═OR″, C═O—OR′″, or C═O—NR″″R″″),
   small alkyl (C1-C10) (optionally C1-C6) (optionally substituted with alkyl, amino, cycloheteroalkyl, cycloalkyl fluoro, aryl, heteroaryl, cycloheteroalkyl, alkylthio, P═O(OR″)₂, CH₂P═O(OR″)₂, CF₂P═O(OR″)₂, NHCOCOOR″, CH(COOR″)₂, arylthio, cyano, OR″, OC═OR″, C═O—OR′″, or C═O—NR″″R″″),
   phenyl and mono and disubstituted (at positions 3 and 4) phenyl (wherein the phenyl ring is independently substituted with alkyl, trifluoromethyl, mono and di halogen atoms, alkylthio, alkoxy, nitro, cyano, rnorphilino, cyclohexyl, phenyl, phenolic, dioxymethylene, nitro, acetylamino, OR′, P═O(OR″)₂, CH₂P═O(OR″)₂, CF₂P═O(OR″)₂, NHCOCOOR″, CH(COOR″)₂), heteroaryl, cycloheteroalkyl and cycloheteroalkyl (each optionally substituted with alkyl, halogen, alkylthio, alkoxy, or nitro, OR′, P═O(OR″)₂, CH₂P═O(OR″)₂, CF₂P═O(OR″)₂, NHCOCOOR″, CH(COOR″)_{2,}),
   cycloalkyl (C3-C10) (optionally substituted with alkyl, fluoro, aryl, heteroaryl, cycloheteroalkyl, cycloheteroalkyl alkylthio, arylthio, cyano, P═O(OR″)₂, CH₂P═O(OR″)₂, CF₂P—O(OR″)₂, NHCOCOOR″, CH(COOR″)₂, OR′, OC═OR′, C═O—OR″, or C═O—NR′″R″″)
   alkenyl (C1-C10) (optionally substituted with alkyl, fluoro, aryl, heteroaryl, cycloheteroalkyl, cycloheteroalkyl, alkylthio, arylthio, cyano, P═O(OR″)₂, CH₂P═O(OR″)₂, CF₂P═O(OR″)₂, NHCOCOOR″, CH(COOR″)₂, OR′, OC═OR′, C═O—OR″, or C═O—NR′″R″″)
   alkadienyl (C1-C10) (optionally substituted with alkyl, fluoro, aryl, heteroaryl, cycloheteroalkyl, cycloheteroalkyl, alkylthio, arylthio, cyano, P═O(OR″)₂, CH₂P═O(OR″)₂, CF₂P—O(OR″)₂, NHCOCOOR″, CH(COOR″)₂, OR′, —OC═OR5, —C═O—OR″, C═O—NR′″R″″)
   cycloalkenyl (C4-C10), optionally substituted with alkyl, fluoro, aryl, heteroaryl, cycloheteroalkyl, cycloheteroalkyl, alkylthio, arylthio, cyano, P═O(OR″)₂, CH₂P═O(OR″)₂, CF₂P═O(OR″)₂, NHCOCOOR″, CH(COOR″)₂, OR′, —OC═OR′, —C═O—OR″, C—O—NR′″R″″
   bicycloalkyl (C5-C12), optionally substituted with alkyl, fluoro, aryl, heteroaryl, cycloheteroalkyl, cycloheteroalkyl, alkylthio, arylthio, cyano, P═O(OR″)₂, CH₂P═O(OR″)₂, CF₂P═O(OR″)₂, NHCOCOOR″, CH(COOR″)₂, OR′, —OC═OR′, —C═O—OR″, C═O—NR′″R″″
   tricycloalkyl (C8-C14), optionally substituted with alkyl, fluoro, aryl, heteroaryl, cycloheteroalkyl, cycloheteroalkyl, alkylthio, arylthio, cyano, P═O(OR″)₂, CH₂P═O(OR″)₂, CF₂P═O(OR″)₂, NHCOCOOR″, CH(COOR″)₂, OR′, —OC═OR′, —C═O—OR″, C—O—NR′″R″″
   where
   Each R′ is independently:
      hydrogen,
      alkyl (C1-C10), optionally substituted with alkyl, keto, fluoro, alkoxy, alkylthio, aryl, heteroaryl, cycloheteroalkyl, cycloheteroalkyl, cyano, aryloxy, cycloalkyl, COOR″, P═O(OR″)₂, CH₂P═O(OR″)₂, CF₂P═O(OR″)₂, NHCOCOOR″, CH(COOR″)₂;
      aryl, optionally substituted with alkyl, keto, fluoro, alkoxy, alkylthio, cyano, aryloxy, COOR″, P═O(OR″)₂, CH₂P═O(OR″)₂, CF₂P═O(OR″)₂, NHCOCOOR″, CH(COOR″)₂;
      heteroaryl, cycloheteroalkyl, cycloheteroalkyl, optionally substituted with alkyl, keto, fluoro, alkoxy, alkylthio, cyano, aryloxy, COOR″, P═O(OR″)₂, CH₂P═O(OR″)₂, CF₂P═O(OR″)₂, NHCOCOOR″, CH(COOR″)₂;
      cycloalkyl, optionally substituted with alkyl, keto, fluoro, alkoxy, alkylthio, aryl, heteroaryl, cycloheteroalkyl, cycloheteroalkyl, cyano, COOR″, P═O(OR″)₂, CH₂P═O(OR″)₂, CF₂P═O(OR″)₂, NHCOCOOR″, CH(COOR″)₂;
   Each R″ is independently
      hydrogen,
      alkyl (C1-C10), optionally substituted with alkyl, keto, fluoro, alkoxy, alkylthio, aryl, heteroaryl, cycloheteroalkyl, cycloheteroalkyl;
      aryl, optionally substituted with alkyl, keto, fluoro, alkoxy, alkylthio;
      heteroaryl, cycloheteroalkyl, optionally substituted with alkyl, keto, fluoro, alkoxy, alkylthio;
      cycloalkyl, optionally substituted with alkyl, keto, fluoro, alkoxy, alkylthio, aryl, heteroaryl, cycloheteroalkyl;
   Each R′″ is independently
      alkyl (C1-C10), optionally substituted with alkyl, keto, fluoro, alkoxy, alkylthio, aryl, heteroaryl, cycloheteroalkyl, COOR″, P═O(OR″)₂, CH₂P═O(OR″)₂, CF₂P═O(OR″)₂, NHCOCOOR″, CH(COOR″)₂;
      aryl, optionally substituted with alkyl, keto, fluoro, alkoxy, alkylthio;
      heteroaryl, cycloheteroalkyl, optionally substituted with alkyl, keto, fluoro, alkoxy, alkylthio, COOR″, P═O(OR″)₂, CH₂P═O(OR″)₂, CF₂P═O(OR″)₂, NHCOCOOR″, CH(COOR″)₂;
      cycloalkyl, optionally substituted with alkyl, keto, fluoro, alkoxy, alkylthio, aryl, heteroaryl, cycloheteroalkyl, COOR″, P═O(OR″)₂, CH₂P═O(OR″)₂, CF₂P═O(OR″)₂, NHCOCOOR″, CH(COOR″)₂; and
   Each R″″ is independently
      alkyl (C1-C10), optionally substituted with alkyl, keto, fluoro, alkoxy, alkylthio, aryl, heteroaryl, cycloheteroalkyl, COOR″, P═O(OR″)₂, CH₂P═O(OR″)₂, CF₂P═O(OR″)₂, NHCOCOOR″, CH(COOR″)₂;
      aryl, optionally substituted with alkyl, keto, fluoro, alkoxy, alkylthio, COOR″, P═O(OR″)₂, CH₂P═O(OR″)₂, CF₂P═O(OR″)₂, NHCOCOOR″, CH(COOR″)₂;
      heteroaryl, cycloheteroalkyl, optionally substituted with alkyl, keto, fluoro, alkoxy, alkylthio, COOR″, P═O(OR″)₂, CH₂P═O(OR″)₂, CF₂P═O(OR″)₂, NHCOCOOR″, CH(COOR″)₂; and
      cycloalkyl, optionally substituted with alkyl, keto, fluoro, alkoxy, alkylthio, aryl, heteroaryl, cycloheteroalky, COOR″, P═O(OR″)₂, CH₂P═O(OR″)₂, CF₂P═O(OR″)₂, NHCOCOOR″, CH(COOR″)₂.
         And wherein each of R1, R2 and R3 are linked to their respective core atoms through C, N, O or S of the substituent group, provided that if R2 is to be linked through O or S, then the core atom S is oxidized.

In the compounds of formula (I), it is preferred that R1 be an aryl group optionally substituted with one or more halogen atoms; R2 be a phenylmethyl group optionally substituted at the 3 or 4 position with one or more aryl, perfluoroalkyl (C1-C4), or thiadiazolyl groups; and R3 be an benzoyl group optionally substituted with one or more perfluoroallyl (C1-C4) substituents.

Specific examples of groups that may be represented by R1 include 3-bromophenyl and 3,4-dichlorophenyl. Specific examples of groups that may be represented by R2 include 4-phenyl phenylmethyl, 4-(1,2,3-thiadiazol-4-yl)-phenylmethyl, and 3-trifluoromethylphenylmethyl. A specific example of a group represented by R3 includes 3-trifluoromethylbenzoyl.

Alternatively, R1 and R2 can be taken together with the core unit to which they are attached (formula I) to form a heterocyclic group having formula (II) as follows: 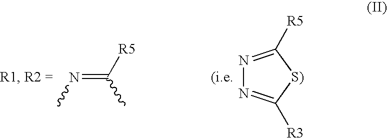
Where R3 is as defined previously.
Of the compounds of formula II, it is preferred that R3 is
(1) a phenyl group optionally substituted with one to three of the following and their combinations: halogen, hydroxy, aryloxy, nitro, carboxylic acid, CF₂P═O(OH)₂, NHCOCOOH, alkyl (C1-C10) or alkoxy (C1-C10) (optionally substituted with NR1R2, COOH, cycloheteroalkyl), alkylthio (C1-C4), 2′-hydroxyethoxy, alkoxycarbonylmethoxy (C1-C4), dialkylamino (C1-C4 where the two alkyls optionally form a heteroalicyclic ring), 2-(dialkylamino)-2-oxoethoxy (C1-C7 where the two alkyls optionally form a heteroalicyclic ring), difluoromethoxy, perfluoroalkyl (C1-C4), perfluoroalkylthio (C1-C4), perfluoroalkoxy (C1-C4), 2-carboxyvinyl, alkanoyl (C1-C5), alkoxycarbonyl (C1-C4), alkanoylamino (C1-C8), benzoylamino (optionally substituted with one or more perfluoroalkyl group (C1-C4) and/or CF₂P═O(OH)₂, NHCOCOOH,), aryl, aryloxy, arylcarbonyl, arylmethoxy, arylmethyl in which the methyl group is substituted with hydroxyl, O(CH₂)ₙCOOH (n=1-5), S(CH₂)ₙCOOH (n-1-5), (4-carboxy)benzyloxy, (3-carboxybenzyloxy), or the group ═N—O—CH₂R in which R is carboxyl, alkoxycarbonyl (C1-C4), hydrogen, or phenyl (optionally substituted with one or more halogens), or the group ═N—NHAr in which Ar is a phenyl (optionally substituted with one or more alkyl groups (C1-C4), and/or a carboxyl group, and/or CF₂P═O(OH)₂, NHCOCOOH), or the group-Y—(CH₂)ₙ-Z, where Y is O or S, n is 1, 2, or 3, and Z is hydrogen, methyl, branched alkyl (C3-C5), cycloalkyl (C3-C6), phenyl (optionally substituted with one or more of the following: halogen, trifluoroalkyl, carboxy, alkoxycarbonyl (C1-C4), CF₂P═O(OH)₂, NHCOCOOH, or carboxyl). It is also preferred that the aryl group in the aryl, aryloxy, arylcarbonyl, arylmethoxy and arylmethyl substituents within R3 will be phenyl or pyridyl (optionally substituted with one or more of the following and their combinations: amino, perfluoroalkyl (C1-C4), CF₂P═O(OH)₂, NHCOCOOH, CH═CH(CH2)ₙCOOH (n=1-5), 1-tetrazolo, 1-tetrazolo substituted at the 2 position with carboxyalkyl (n=1-5), N-(4-carboxyphenylamino)iminomethyl, perfluoroalkoxy (C1-C4), alkyl (C1-C10) or alkoxy (C1-C10) (optionally substituted with NR1R2, COOH, cycloheteroalkyl), alkoxycarbonyl (C1-C4), carboxy, nitro, carboxyalkyl (C1-C7), carboxyalkenyl (C1-C7), formyl, hydroxyalkyl (C1-C4), halogen, hydroxy, cyano, tetrazole (optionally substituted with alkyl (C1-C4), carboxyalkyl (C1-C4), or alkoxycarbonylmethyl (C1-C4)), CONHCH₂(CHOCH₂CH₂O), or hydroxyalkyl (C1-C4); or
(2) a pyridylthio group optionally substituted with one or more halogen and/or one or more nitro groups, methylenedioxyphenyl, benzo[3,4-c]1,2,5-oxadiazol-5-yl, 4-oxo-3-hydroquinazolin-2-yl, or a group having formula (III) as follows: 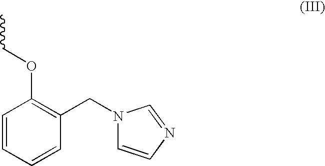
   in which the imidazole ring is optionally substituted with one or more halogens, and where R5 is as defined for R1, R2 and R3 above.
   Of these, it is preferred that R5 is an amino group with two substituents, where one substituent is arylcarbonyl, arylmethylcarbonyl, arylsulfonyl, aryldimethyloxycarbonyl, or aryloxymethylcarbonyl, [where the aryl group is phenyl, benzox[c]1,2,5-oxadiazol-5-yl, 1-furyl, 2-furyl 1-naphthyl or 2-naphthyl, optionally substituted with one or more of the following or their combinations: perfluoroalkyl (C1-C4), alkyl (C1-C4), nitro, alkoxycarbonyl (C1-C4), carboxyl, carboxyalkyl(C1-C4), CF₂P═O(OH)₂, NHCOCOOH, phenoxy (optionally substituted with alkoxy (C1-C4), CF₂P═O(OH)₂, NHCOCOOH, COOH, and/or halogen), or phenylalkoxy (C1-C4)], hydrogen, CF₂P═O(OH)₂, NHCOCOOH, or a phenyl group [optionally with one or more of the following substituents or combinations: hydroxy, halogen, nitro, CF2P═O(OH)₂, NHCOCOOH, carboxy, carboxyalkyl(C1-C4), carboxyalkylthio (C1-C6), phenyl, alkyl (C1-C10) or alkoxy (C1-C10) (optionally substituted with NR1R2, COOH, cycloheteroalkyl), perfluoroalkyl (C1-C4), alkoxycarbonyl (C1-C4), alkyllhio (C1-C4), phenylalkoxy (C1-C4), phenylsulfonylamino (each optionally substituted on phenyl with alkyl (C1-C4)), phenoxy (optionally substituted on phenyl with nitro, perfluoroalkyl (C1-C4), carboxymethyl, carboxy, CF₂P═O(OH)₂, NHCOCOOH, alkoxycarbonylmethyl (C1-C4)), carboxyalkyl(C1-C4), phenylalkylthio (C1-C4, optionally substituted on phenyl with alkoxy (C1-C4), and/or phenyl), aminosulfonyl, alkylaminosulfonyl (C1-C4), dialkylaminosulfonyl (C1-C4 where the two alkyls optionally form a heteroalicyclic ring).] The second substituent on the amino group forming R5 is hydrogen, alkyl (C1-C10) or alkoxy (C1-C10) (each optionally substituted with NR1R2, COOH, CF₂P═O(OH)₂, NHCOCOOH, cycloheteroalkyl), naphthylalkyl (C1-C4), phenylalkyl (C1-C4, with the phenyl group optionally substituted with phenyl, alkyl (C1-C4), halo, amino, amido, keto, CF₂P═O(OH)₂, NHCOCOOH, alkyl (C1-C10) or alkoxy (C1-C10) (optionally substituted with NR1R2, COOH, cycloheteroalkyl), nitro, carboxy, perfluoroalkylthio (C1-C4), halogen, CF₂P═O(OH)₂, NHCOCOOH, 1,2,3-thiadiazolyl, and/or alkoxy carbonyl (C1-C4)), alkyl (C1-C10), cycloalkyl (C4-C8, optionally substituted with alkyl (C1-C4)), or indanyl (optionally substituted with alkyl (C1-C4)). It is also preferred that R5 is a phenyhmethylthio group, optionally substituted with one or more halogens on the phenyl ring, or 2-oxo-2-(2-naphthylethylthio) optionally substituted with one or more alkyl groups (C1-C4).

Specific examples of groups that may be represented by R3 include: 3-nitrophenyl, 3,5-dinitrophenyl, 3,4-dihydroxyphenyl, 2-chlorophenyl, 2-trifluoromethylphenyl, 3-carboxyphenyl, 3-methylphenyl, 3-methoxyphenyl, 3-ethoxyphenyl, 3-trifiuoromethoxyphenyl, 4-carboxyphenylmethyl, 3-(3-(N-(4-carboxyphenylamino)iminomethyl)phenoxy)phenyl, 3-(3-(6-carboxy-hex-1-enyl)phenoxy)phenyl, 3-(3-carboxyphenylmethoxy)-5-(phenylmethoxy)phenyl, 3-(3-carboxyphenoxy)phenyl, 3,5-bis(phenylmethoxy)phenyl, 3,5-bis(3-methoxyphenylmethoxy)phenyl, 3,5-bis(3-trriluoromethylphenylmethoxy)phenyl, 3-(3-((2-carboxybutyl)-1-tetrazolo))phenoxy)phenyl, 3-((4-carboxyphenylamino)imino)(3-trifluoromethylphenyl)methyl)phenyl,-3-((carboxymethylthio)(3-trifluoromethylphenyl)methyl)phenyl, 3-(3-(1-hydroxy, 2,2 difluoro, 2-(dihydroxyphosphono)ethyl)phenoxy)phenyl, 3-trifluoromethylthiophenyl, 3-(4-methylpentanoylamino)phenyl), 4-dimethylaminophenyl, 4-ethoxyphenyl, 4-methylthiophenyl, 4-difluoromethoxyphenyl, 4-(2-carboxyvinyl)phenyl, 3,4-dinethoxyphenyl, 3-chloro-4-bromophenyl, 3-bromo-4-chlorophenyl, 3-nitro-4-morpholinophenyl, 3-phenylmethoxyphenyl, 3-[2-(4-methylpiperidyl)-2-oxoethoxy]phenoxy, 3-(1-naphthylsulfonylamino)phenyl, 4-phenylphenyl, 3,5-dimethoxyphenyl, 3,4-methylenedioxyphenyl, 3-phenylmethoxy-4-methoxyphenyl, benzo[3,4-c]1,2,5-oxadiazol-5-yl, 4-oxo-3-hydroquinazolin-2-yl, 3-(2′-hydroxyethoxy)phenyl, 3-(ethoxycarbonylmethoxy)phenyl, 3-n-butoxyphenyl, 4-ethylaminophenyl, 4-phenoxyphenyl, 3-(3′-trifluoromethylphenoxy)phenyl, 3-(3′aminophenoxy)phenyl, 3,5-bis(3′trifluoromethylphenoxy)phenyl, 6-(3′trifluoromethylphenoxy)-2-pyridyl, 3-(3′methoxycarbonylphenoxy)phenyl, 3-(3′trifluoromethylphenoxy)-5-hydroxyphenyl, 3-(3′-hydroxyphenoxy)phenyl, 3-(3′-(1-ethoxycarbonylmethyl-1,2,3,4-tetrazol-5-yl)phenoxy)phenyl, 3-(4′-t-butylphenoxy)phenyl, 3-(3′carboxyphenoxy)phenyl, 3-(3′nitrophenoxy)phenyl, -(3′carboxymethylphenoxy)phenyl, 3-(4′methoxyphenoxy)phenyl, 3-(4′methylphenoxy)phenyl, 3-(3′,5′-dichlorophenoxy)phenyl, 3-(3′,4′-dichlorophenoxy)phenyl, 3-(4′hydroxyphenoxy)phenyl, 3-(3′cyanophenoxy)phenyl, -3-(3′-(tetrazole-1-yl)phenoxy)phenyl, 3-(3′-(N-(1,3-dioxolan-2-ylmethyl)carbamoyl)phenoxy)phenyl, 3-(3′-hydroxymethylphenoxy)phenyl, 3-(3′(2″-carboxyethyl)phenoxy)phenyl, 3-(4′-t-butylphenylmethoxy)phenyl, 4-(3′,5′-bis(trifluoromethyl)benzoylamino)phenyl, 3-(3′-trifluoromethoxyphenylmethoxy)phenyl, 3-(3′-trifluorophenylmethoxy)phenyl, 3-(3′-trifluoromethylbenzoyl)phenyl, 3-((3′-trifluoromethylphenyl)hydroxymethyl)phenyl, 3-[3′-[2-aza-2-(ethoxycarbonylmethoxy)vinyl]phenoxy]phenyl, 3-[3′-[2-aza-2-(carboxymethoxy)vinyl]phenoxy]phenyl, 3-[3′-[2-aza-2-(4″-carboxyphenylamino)vinyl]phenoxy]phenyl, 3-{ethoxy [3-(trifluoromethyl)phenyl]methyl}phenyl, 3-{(carboxymethoxy)[3-(trifluoromethyl)phenyl]methyl}phenyl, 3-{(ethoxylcarbonylmethoxy)[3-(trifluoromethyl)phenyl]methyl}phenyl, 3-{(ethoxylcarbonylmethyl)[3-(trifluoromethyl)phenyl]methyl}phenyl, 3-{(carboxymethyl)[3-(trifluoromethyl)phenyl]methyl}phenyl, 3-{(2′,4′-dichlorophenylmethylthio)[3-(trifluoromethyl)phenyl]methyl}phenyl, 3-(1-(3′-trifluoromethylphenyl)-2-aza-2-(4″-carboxyphenylamino)vinyl)phenoxy, 3-(3′-(6-carboxyhex-1-enyl)phenoxy)phenyl, 3-{(3-methyl-1-butylthio)[3-(trifluoromethyl)phenyl]methyl}phenyl, 3-{(2-carboxyethylthio)[3-(trifluoromethyl)phenyl]methyl}phenyl, 3-{(ethoxycarbonylmethoxy)[3-(trifluoromethyl)phenyl]methyl}phenyl, 3,5-bis(phenylmethoxy)phenyl, (4,5-dichloroimidazolyl)methyl]phenoxy}methyl), 2-nitro-4-bromo-(2-pyridylthio).

Specific examples of groups that may be represented by R5 include 3-bromophenylamino, 4-bromophenylamino, 4-fluorophenylamino, 3-nitrophenylamino, 4-nitrophenylamino, 3-fluorophenylamino, 4-aminosulfonylphenylamino, 3-methylphenylamino, 3-hydroxyphenylamino, 3-carboxyphenylamino, 4-ethoxycarbonylphenylamino, 3-methoxyphenylamino, 3-methoxycarbonylphenylamino, 4-carboxyphenylamino, 3-trifluoromethylphenylamino, 4-acetylphenylamino, 4-ethylphenylamino, 4-isopropylphenylamino, 3,5-dinitrophenylamino, 4-(n-butyl)-phenylamino, 4-(n-decyl)amino, 4-ethoxycarbonylphenylamino, 4-methoxycarbonylphenylamino, 3,(4-carboxymethylphenoxy)phenylamino, 4-piperidinosulfonylphenylamino, 4-(4-nitrophenoxy)phenylamino, 3-(4-phenylphenylmethylthio)phenylamino, 3-(2-phenylphenylmethylthio)phenylamino, 3-carboxymethylphenylamino, 3-(2-carboxyethyl)phenylamino, 3-(3-trifluoromethylphenoxy)phenylamino, naphthalene-1-sulfonylamino, naphthalene-2-sulfonylamino, 3-(4-methoxylphenylmethylthio)phenylamino, 3-(3-phenylpropylthio)phenylamino, 2,5-dibromophenylamino, 3-chloro-4-fluorophenylamino, 2,3-dichlorophenylamino, 3,4-dichlorophenylamino, 3,4-dibromophenylamino, 2-chloro-5-nitrophenylamino, 2,4-dimethoxy-5-chlorophenylamino, 3-chloro-4-methylphenylamino, 3-chloro-4-bromophenylamino, 3-methyl-4-bromophenylamino, 4-(phenylsulfonylamino)phenylamino, 4-(4′-methylphenylsulfonylamino)-phenylamino, (3-chloro-4-bromophenyl)(2-phenylmethyl)amino, (3-chloro-4-bromophenyl)(2-trifluoromethylthiophenylmethyl)amino, (3-chloro-4-bromophenyl)(4-t-butylphenylmethyl)amino, (2,3,4,5-tetrachlorophenyl)(4-t-butylphenylmethyl)amino, (3,4-dichlorophenyl)(3-methoxycarbonylphenyl)methyl)amino, (3,4-dichlorophenyl)(4-methoxycarbonylphenyl)methyl)amino, (3,4-dichlorophenyl)(3-carboxyphenyl)methyl)amino, 3-phenylmethoxyphenylamino, 3-chloro-4-methylphenylamino, 2,3,4,5-tetrachlorophenylamino, (3,4-dichlorophenyl)[(4-carboxyphenyl)methyl]amino, (3,4-dichlorophenyl)[(4-methoxycarbonylphenyl)methyl]amino, (phenylmethyl)(3-chloro-4-bromophenyl)amino, (phenylmethyl)(3-bromophenyl)amino, (3,4-dichlorophenyl)[(4-phenylphenyl)methyl]amino, (3,4-dichlorophenyl)[4-(t-butylphenyl)methyl]amino, (3,4-dichlorophenyl)[(3-nitrophenyl)methyl]amino, (3,4-dichlorophenyl)[2-naphthylmethyl]amino, (methyl)(3,4-dichlorophenyl)amino, 3-trifluoromethylbenzoylamino, 3,5-bistrifluoromethylbenzoylamino, 3-methoxycarbonylbenzoylamino, 3-nitrobenzoylamino, 4-t-butylphenoxyacetylamino, 2-(4-chlorophenoxy)-2-methylpropanoylamino, benzo[c]1,2,5-oxadiazol-5-yl-amino, 2-(4-methoxyphenoxy)-5-nitrobenzoylamino, 5-(3,5-dichlorophenoxy)-2-furoylamino, 2-methyl-3-chlorobenzoyl, 4-(phenylmethoxy)phenylacetylarmino, 1-naphthoylamino, 2-(4-methylphenyl)ethylamino, 1-napthylmethylamino, 2-indanylamino, 3,3,5-trimethylcyclohexylamino, 2-heptylamino, 3,4-dichlorophenylmethylthio, 2,6-dichlorophenylmethylthio, and 2-oxo-2-(2-naphthyl)ethylthio.

Alternatively, R1 and R2 can be taken together with the core unit to which they are attached (formula I) to form a heterocyclic group having formula (IV) as follows: 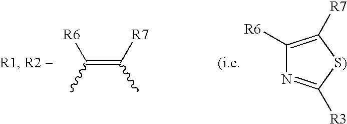
Wherein R3 is as defined previously.

Of these, it is preferred that that R3 be an arylamino group in which the aryl group is phenyl or pyridyl (optionally substituted with one or more of the following groups: phenyl, halogen, or hydroxy), or a phenylamino group (optionally substituted on the phenyl with one or more of the following: halogen, phenoxy, perfluoroalkyl (C1-C4), alkyl (C1-C4), or nitro), or a phenyl group optionally substituted with one or more nitro groups.

Specific examples of groups that may be represented by R3 include 2-hydroxy-5-chlorobenzoylamino, 2-hydroxy-5-bromobenzoylamino, 3-pyridinecarboxylamino, 4-bromobenzoylamino, 2-nitro-5-chlorobenzoylamino, 2,6-dimethoxy-3,5-dichlorobenzoylamino, 3-bromophenylamino, 4-phenoxyphenylamino, 3,4-dichlorophenylamino, 2,4,5-trichlorophenylamino, 3,5-dichlorophenylamino, bis(trifluoromethyl)phenylamino, and 3-nitrophenyl.

Wherein R6 is as defined above for R1, R2 and R3.

Of these, it is preferred that that R6 be hydrogen, naphthyl, or phenyl [optionally substituted with one or more of the following: phenyl, alkoxy (C1-C4), alkyl (C1-C4), nitro, cyano, halogen, dialkylamino (C1-C4, with the two alkyl groups optionally joined to form a heterocycle), alkoxycarbonyl (C1-C4), benzoyloxy].

Specific examples of groups that may be represented by R6 include hydrogen, 4-phenylphenyl, 3-methoxyphenyl, 4-methylphenyl, 4-nitrophenyl, 4-cyanophenyl, 3-chloro-4-methylphenyl, 3,4-dichlorophenyl, 3-methyl-4-chlorophenyl, 4-diethylaminophenyl, 4-N-pyrrolidinophenyl, 2-(ethoxycarbonyl)phenyl, 3-benzoyloxyphenyl, 4-benzoyloxyphenyl, 2-naphthyl.

Wherein R7 is as defined above for R1, R2 and R3.

Of these, it is preferred that that R7 be hydrogen, alkyl (C1-C4), benzoyl (optionally substituted with one or more of the following or their combinations: halogen, nitro, alkoxy (C1-C4)), phenyl (optionally substituted with one or more halogen or nitro group), phenylamino (optionally substituted with one or more halogens), or 2H,3H,4H-benzo[3,4-b]1,4-dioxepan-7-yl (optionally substituted with one or more alkyl(C1-C4)).

Specific examples of groups that may be represented by R7 include hydrogen, methyl, benzoyl, 4-bromobenzoyl, 3,4-dichlorobenzoyl, 2-nitrophenyl, 3-nitrophenyl, 4-chlorophenyl, 2H,3H,4H-benzo[3,4-b]1,4-dioxepan-7-yl, and 3-bromophenylamino.

Alternatively, R1 and R2 are linked through an aromatic ring, and taken together with the N═CR3—S unit to which they are attached, form a tricyclic heterocyclic group having formula (V) as follows: 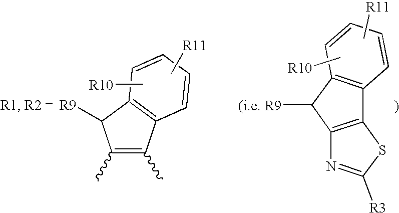
Where R3 is as defined above for R1, R2 and R3.
Where R9 is as defined above for R1, R2 and R3.
Where R10 is as defined above for R1, R2 and R3.
Where R11 is as defined above for R1, R2 and R3.

Of these, it is preferred that that R3 be phenylamino (optionally substituted on phenyl with one or more of the following: halogen, alkyl(C1-C4), perfluoroalkyl(C1-C4)). It is preferred that that R9 be hydrogen or alkyl(C1-C4). It is preferred that that R10 and R11, independently, be H, alkyl (C1-C4), or halogen. In a specific example, R3 is 2,4,5-trichlorophenylamino, and R9, R10, and R11 are hydrogen.

Alternatively, R1 and R2, taken together with the N═CR3—S unit to which they are attached, form a heterocyclic group having formula (VI) as follows: 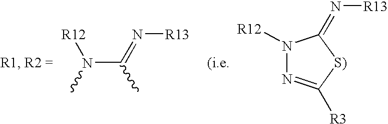
Where R1 is as defined above for R1, R2 and R3.
Where R12 is as defined above for R1, R2 and R3.
Where R13 is as defined above for R1, R2 and R3.
Of these, it is preferred that that R3 be phenyl, optionally substituted with one or more of the following: Halogen, nitro, alkyl (C1-C10), CF₂P═O(OH)₂, or alkoxy (C1-C10) (optionally substituted with NR1R2, COOH, cycloheteroalkyl), phenoxy (optionally substituted with perfluoroalkyl(C1-C4), carboxy, carboxymethyl, N-(4-carboxyphenylamino)iminomethylene, CF₂P═O(OH)₂, alkyl (C1-C10) or alkoxy (C1-C10) (optionally substituted with NR1R2, COOH, cycloheteroalkyl), and/or halogen). It is preferred that that R12 be alkyl (C1-C10, optionally substituted with carboxyl or CF₂P═O(OH)₂,) or alkoxy (C1-C10) (optionally substituted with NK1R2, COOH, cycloheteroalkyl), naphthylalkyl(C1-C4), or phenylalkyl(C1-C4, optionally substituted on phenyl with carboxyalkyl, carboxy, CF₂P═O(OH)₂, phenyl, alkyl (C1-C10) or alkoxy (C1-C10) (optionally substituted with NR1R2, COOH, cycloheteroalkyl), or alkoxycarbonylalkyl(C1-C4)). It is preferred that that R13 be branched alkyl (C1-C5), alkyl (C1-C5), cycloalkyl (C3-C7), phenyl (optionally substituted with one or more of the following or their combinations: halogen, alkoxycarbonyl(C1-C4), alkyl (C1-C10), piperidinosulfonyl, or alkoxy (C1-C10) (optionally substituted with NR1R2, COOH, cycloheteroalkyl)), cycloalkyl, alkyl (C1-C10) or alkoxy (C1-C10) (optionally substituted with NR1R2, COOH, cycloheteroalkyl), heteroaryl, and cycloheteroaryl. Specific examples of R3 are 3-nitrophenyl, 3-ethoxyphenyl, 3-phenoxyphenyl, 3-(3-carboxyphenoxy)phenyl, 4-carboxyphenyl, 3-(3-(N-(4-carboxyphenylamino)iminomethyl)phenoxy)phenyl, 3-(4-(dihydroxyphosphonodifluoromethyl)phenoxy)phenyl, and 3-(3-trifluoromethylphenoxy)phenyl. Specific examples of R12 include methyl, phenylmethyl, 3-methoxyphenylmethyl, 3-(methoxycarbonyl)phenylmethyl, 2-trifluoromethylphenylmethyl, 4-carboxyphenylmethyl, 4-(carboxymethyl)phenylmethyl, carboxylmethyl, 4-(dihydroxyphosphonodifluoromethyl)-butyl, 4-(dihydroxyphosphonodifluoromethyl)phenylmethyl, 4-(1,2,3-thiadiazole-4-yl)phenylmethyl, 4-t-butylphenylmethyl, 3-methoxycarbonylphenyl, 4-methoxycarbonylphenyl, 2-naphthylmethyl, and 4-phenylphenylmethyl. Specific examples of R13 include 3-bromophenyl, 3,4-dichlorophenyl, 3-chloro-4-bromophenyl, isopropyl, 4-(piperidinosulfonyl)phenyl, 3-(3-trifluoromethylphenoxy)phenyl, and 3-methoxycarbonylphenyl.

Alternatively, R1 and R3, taken together with the N═C—SR2 unit to which they are attached, form a heterocyclic group having formula (VII) as follows: 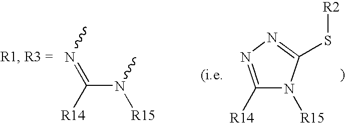
Where R2 is as defined above for R1, R2 and R3.
Where R14 is as defined above for R1, R2 and R3.
Where R15 is as defined above for R1, R2 and R3.
Of these, it is preferred that that R2 be 2-phenyl-2-oxoethylthio, optionally substituted on phenyl with one or more of the following or their combinations: nitro, halogen, alkyl (C1-C4). It is preferred that that R14 be phenyl, optionally substituted with one or more alkyl groups (C1-C6). It is preferred that that R15 be hydrogen or alkyl(C1-C4). A specific example of R2 is 2-(4-nitrophenyl)-2-oxoethylthio. A specific example of R14 is 4-n-pentylphenyl. A specific example of R15 is hydrogen.

Alternatively, R1 and R3, taken together with the N═C—SR2 unit to which they are attached, form a bicylic heterocyclic group having formula (VIII) as follows: 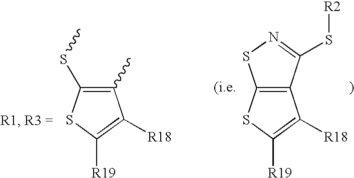
Where R2 is as defined above for R1, R2 and R3.
Where R18 is as defined above for. R1, R2 and R3.
Where R19 is as defined above for R1, R2 and R3.
Of these, it is preferred that that R2 be 2-phenyl-2-oxyethyl, optionally substituted on phenyl with one or more of the following or their combinations: halogen, alkyl (C1-C4). It is preferred that that R18 be amino, optionally substituted with one or two alkyl groups (C1-C4). It is preferred that that R19 be benzoyl, optionally substituted with one or more of the following or their combinations: halogen, alkyl (C1-C4).

Alternatively, R1, R2 and R3, taken together with the N═C—S unit to which they are attached, form a bicyclic heterocyclic group having formula (IX) as follows: 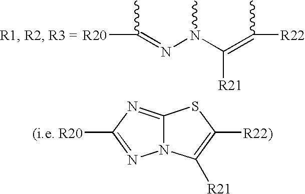
Where R20 is as defined above for R1, R2 and R3.
Where R21 is as defined above for R1, R2 and R3.
Where R22 is as defined above for R1, R2 and R3.
Of these, it is preferred that that R20 be phenyl, optionally substituted with one or more of the following or their combinations: halogen, alkyl (C1-C4). It is preferred that that R21 be hydrogen, alkyl (C1-C4), or phenyl, optionally substituted with one or more of the following or their combinations: hydroxy, alkyl (C1-C4). It is preferred that that R2 be hydrogen, phenylthioacyl (optionally substituted with one or more halogens), phenylaminoacylamino (optionally substituted on phenyl with one or more halogens), phenylhydrazinoacylamino (optionally substituted on phenyl with one or more halogens).
A specific examples of R20 is 4-chlorophenyl. Specific examples of R21 are methyl or 2,4-dihydroxyphenyl. Specific examples of R22 are hydrogen, 2,4-difluorophenylthioacyl, phenylaminocarbonylamino, 2,4-dichlorophenylaminocarbonylamino, and 2,4-dichlorophenylhydrozinocarbonylamino.

Alternatively, R1, R2 and R3, taken together with the N═C—S unit to which they are attached, form a bicyclic heterocyclic group having formula (X) as follows: 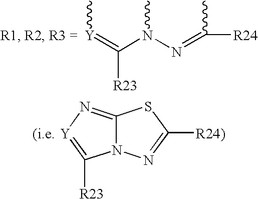
Where R23 is as defined above for R1, R2 and R3.
Where R24 is as defined above for R1, R2 and R3.
Of these, it is preferred that that Y be nitrogen or carbon substituted with an aromatic group which consists of phenyl (optionally substituted with one or more of the following or their combinations: halogen, phenyl, alkoxy (C1-C4)), phenylisoxazolyl, optionally substituted with one or more halogens, or 2H,3H,4H-benzo[3,4-b]1,4-dioxepan-7-yl, optionally substituted with one or more alkyl groups (C1-C4). It is preferred that that R23 be hydrogen, alkyl (C1-C4), or phenyl, optionally substituted with one or more halogens. It is preferred that that R24 be phenyl, optionally substituted with one or more of the following: halogen, nitro, alkoxy (C1-C4), or alkyl (C1-C4).
Specific examples of Y include nitrogen and carbon substituted with 4-bromophenyl, 4-chlorophenyl, 4-phenylphenyl, 3-(2,4-dichlorophenyl)isoxazol-5-yl], and 2H,3H,4H-benzo[3,4-b]1,4-dioxepan-7-yl. Specific examples of R23 include hydrogen, 4-chlorophenyl, or in which R1 and R2, together with the N═CR3—S unit to which they are attached, form a bicyclic heterocyclic group as follows: 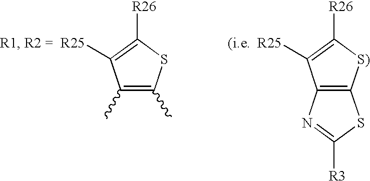
Where R25 is as defined above for R1, R2 and R3.
Where R26 is as defined above for R1, R2 and R3.
Of these, it is preferred that that R3 be benzoylamino, optionally substituted on the phenyl ring with one or more of the following or their combinations: halogen, alkyl (C1-C4), and optionally substituted on nitrogen with alkyl (C1-C4). It is preferred that that R25 be phenyl, optionally substituted with one or more of the following or their combinations: halogen, alkyl (C1-C4). It is preferred that that R26 be perfluoroalkyl (C1-C4).
A specific example of R3 is 4-chlorobenzoylamino. A specific example of R25 is phenyl. A specific example of R26 is trifluoromethyl.

The compounds of the present invention generally contain one or more asymmetric centers and thus give rise to optical isomers and diastereomers. The scope of the present invention includes all possible isomers and diastereomers, as well as their racemic and resolved, enantiomerically pure forms.

Certain of the present compounds contain olefinic double bonds and, unless specified to the contrary, the compounds of the present invention include both the E and Z geometric isomeric forms.

Pro-Drug Compounds of the Invention

Alternatively, the compounds of the present invention can be further modified to act as prodrugs. It is a well-known phenomenon in drug discovery that compounds such as enzyme inhibitors can display potency and selectivity in in vitro assays, yet not readily manifest the same activity in vivo. This lack of “bioavailability” may be due to a number of factors, such as poor absorption in the gut, first-pass metabolism in the liver, and poor uptake in the cells. Although the factors determining bioavailability are not completely understood, there are many techniques known by those skilled in the art to modify compounds, which are potent and selective in biochemical assays but show low or no activity in vivo, into drugs that are biologically and therapeutically active.

It is considered to be within the scope of the invention to modify any of the compounds of the invention (termed the ‘original compound’) by attaching chemical groups that will improve the bioavailability of the original compound. Examples of said modifications include changing of one or more carboxy groups to esters (for instance methyl esters, ethyl esters, acetoxymethyl esters or other acyloxy-methyl esters). Compounds of the invention so modified by attaching chemical groups are termed ‘modified compounds.’

Other examples of modified compounds are compounds that have been cyclized at specific positions (‘cyclic compounds’) which upon uptake in cells or mammals become hydrolyzed at the same specific position(s) in the molecule to yield the compounds of the invention, the original compounds, which are then said to be ‘non-cyclic’. For the avoidance of doubt, it is understood that the latter original compounds in most cases will contain other cyclic or heterocyclic structures that will not be hydrolyzed after uptake in cells or mammals.

Generally, said modified compounds will not show a behavior in biochemical assays similar to that of the original compound, i.e., the corresponding compounds of the invention without the attached chemical groups or said modifications. Said modified compounds may even be inactive in biochemical assays. However, after uptake in cells or mammals these attached chemical groups of the modified compounds may in turn be removed spontaneously or by endogenous enzymes or enzyme systems to yield compounds of the invention, original compounds. ‘Uptake’ is defined as any process that will lead to a substantial concentration of the compound inside cells or in mammals. After uptake in cells or mammals and after removal of said attached chemical group or hydrolysis of said cyclic compound, the compounds may have the same structure as the original compounds and thereby regain their activity and hence become active in cells and/or in vivo after uptake.

A number of techniques well known to those skilled in the art may be used to verify that the attached chemical groups have been removed or that the cyclic compound has been hydrolyzed after uptake in cells or mammals. One example of such techniques is as follows: A mammalian cell line, which can be obtained from the American Type Culture Collection (ATCC) or other similar governmental or commercial sources, is incubated with a modified compound. After incubation under appropriate conditions, the cells are washed, lysed and the lysate is isolated. A number of different procedures, well known to those skilled in the art, may in turn be used to extract and purify the modified compound (or a metabolite thereof) (the ‘purified compound’) from the lysate. The modified compound may or may not retain the attached chemical group or the cyclic compound may or may not have been hydrolyzed. Similarly, a number of different procedures may be used to structurally and chemically characterize the purified compound. Since the purified compound has been isolated from said cell lysate and hence has been taken up by said cell line, a comparison of the structurally and chemically characterized compound with that of the original compound (i.e. without the attached chemical group or other modification) will provide information on whether the attached chemical group as been removed in the cell or if the cyclic compound has been hydrolyzed.

As a further analysis, the purified compound may be subjected to enzyme kinetic analysis as described in detail in the present description. If the kinetic profile is similar to that of the original compound without the attached chemical group, but different from the modified compound, this result confirms that the chemical group has been removed or the cyclic compounds has been hydrolyzed. Similar techniques may be used to analyze compounds of the invention in whole animals and mammals.

A preferred form of prodrug is acetoxymethyl esters of the compounds of the present invention, which may be prepared by the following general procedure (C. Schultz et al., J. Biol. Chem. 1993, 268:6316-6322):

A carboxylic acid (1 eq) is suspended in dry acetonitrile (2 mL/0.1 mmol). Diisopropyl amine (3.0 eq) is added followed by bromomethyl acetate (1.5 eq). The mixture is stirred under nitrogen overnight at room temperature. Acetonitrile is removed under reduced pressure to yield an oil, which is diluted in ethylacetate and washed with water (3×). The organic layer is dried over anhydrous magnesium sulfate. Filtration, followed by solvent removal under reduced pressure, affords a crude oil. The product is purified by column chromatography on silica gel, using an appropriate solvent system.

As used herein, the term “attached” (or “−” or “bound”) signifies a stable covalent bond.

As used herein, the term “alkyl” includes a straight or branched chain saturated hydrocarbon group having from 1 to 20 carbons such as methyl, ethyl, isopropyl, n-butyl, s-butyl, t-butyl, n-amyl, isoamyl, n-hexyl, n-octyl and n-decyl, and includes such cyclic and alkyl-substituted cyclic alkyls that are possible within the given carbon number limitations.

As used herein, the terms “alkenyl” and “alkynyl” include straight or branched chain hydrocarbon groups having from 2 to 10 carbons and unsaturated by a double or triple bond respectively, such as vinyl, allyl, propargyl, 1-methylvinyl, but-1-enyl, but-2-enyl, but-2-ynyl, 1-methylbut-2-enyl, pent-1-enyl, pent-3-enyl, 3-methylbut-1-ynyl, 1,1-dimethylallyl, hex-2-enyl and 1-methyl-1-ethylallyl.

As used herein, the term “phenylalkyl” includes the aforementioned alkyl groups substituted by a phenyl group such as benzyl, phenethyl, phenopropyl, 1-benzylethyl, phenobutyl and 2-benzylpropyl.

As used herein, the term “aryl” includes a monocyclic or bicyclic rings, wherein at least one ring is aromatic including aromatic hydrocarbons or hetero-aromatic hydrocarbons.

As used herein, the term “hydroxy-alkyl” includes the aforementioned alkyl groups substituted by a single hydroxyl group such as 2-hydroxybutyl, 2-hydroxypropyl, 3-hydroxypropyl, 4-hydroxybutyl, 1-hydroxybutyl and 6-hydroxyhexyl.

As used herein, the terms “alkylthio, alkenylthio, alkynylthio, alkylthio, hydroxy-alkylthio and phenyl-alkylthio” mean the aforementioned alkyl, alkenyl, alkynyl, hydroxy-alkyl and phenyl-alkyl groups linked through a sulfur atom to group R.

As used herein, the term “substituted” means that the group in question, e.g., alkyl group, aryl group, etc., may bear one or more substituents including but not limited to halogen, hydroxy, cyano, amino, nitro, mercapto, carboxy and other substituents known to those skilled in the art.

As used herein, the term “saturated” means an organic compound with neither double nor triple bonds, and the term “unsaturated” means an organic compound containing either double or triple bonds.

Procedures for the Synthesis of Compounds and Intermediates

Procedure A

(3-[(2-Phenylphenyl) methylthio] phenylamine): 2-phenyl benzylbromide (2.47 g; 10 mmol) is added slowly to a stirred solution of 3-aminotiophenol (1.25 g; 10 mmol) in the mixture of ethanol (20 mL) and 1M NaOH (10 mL). The mixture is stirred for 30 minutes, the solvent evaporated and the residue is purified using a Biotage column. The product is eluted with EtoAc/Hexanes (4/1). Yield: 1.8 g (62%).

¹H NMR: (300 MHz, CDCl₃) 7.39δ (7H, m); 7.31δ (3H, m); 7.00δ (1H, t); 6.49δ (1H, s); 6.48δ (1H, d); 4.07δ (2H, s).

Procedure B

A mixture of 3-bromobenzaldehyde (1.000 g; 5.4 mmol), methyl 3-hydroxybenzoate (987 mg; 6.5 mmol) and potassium carbonate (1.494 g; 10.8 mmol) in dry pyridine (8 mL) is stirred under argon at RT. Copper (II) oxide (860 mg; 10.8 mmol) is added and the reaction mixture is refluxed for 12 hours. After cooling to RT, CH₂Cl₂ (SOmL) is added and the mixture is filtered through celite. The filter cake is washed with fresh CH₂Cl₂ (50 mL). The combined organics are concentrated in vacuo. The residue is purified by flash chromatography (ethyl acetate/hexanes, 1:10 to 1:4) to yield methyl 3-(3-carbonylphenoxy)benzoate (776 mg; 56%) as a yellow oil.

¹H NMR (300 MHz, CDCl₃): δ 9.97 (1H, s), 7.85 (1H, d, J=6.9 Hz), 7.69-7.24 (7H, m), 3.91 (3H, s).

Procedure C

3-[(2-phenylphenyl) methylthio]benzeneisothiocyanate: Thiophosgene (1.37 g; 12 mmol) is slowly added to a solution of 3-[(2-Phenylphenyl) methylthio]phenyl amine in a mixture of water (60 mL) and Methylenechloride (45 mL). The mixture is stirred under nitrogen at RT for two hours. The aqueous layer is separated, and the methylenechloride is washed with water. The organic layers are dried over anhydrous sodium sulfate and rotovaped to provide a brown oil. Yield: 2.2 g (70%).

Procedure D

The hydrazine hydrate (7.12 mL; 147 mmol) is dissolved in 220 mL of ethanol. This solution is stirred at 0° C. and 3,4-dichlorobenzenisothiocyanate (20.00 g; 98 mmol) is added dropwise, and the reaction mixture is warmed to RT and stirred for two hours. After being cooled to 0° C., the mixture is filtered and the solid washed by cold ethanol (40 mL). The solid is crystallized from ethanol to yield ([(3,4-dichlorophenyl)amino]hydrazinomethane-1-thione) (12.702 g; 55%) as a white solid.

¹H NMR (300 MHz, d₆-DMSO): δ 9.40 (1H, s), 8.19 (1H, s), 7.69 (1H, d), 7.53 (1H, d), 5.30 (3H, br s).

Procedure E

(3-(1,3-Dioxolan-2-yl)phenyl)[3-(trirluoromethyl)phenyl]methan-1-ol) A 1.0 mL aliquot of 2-(3-bromophenyl)-1,3-dioxolane is added into magnesium (610 mg; 25 mmol) and THF (5 mL) under Argon. After the reaction is started, the residue of 2-3-bromophenyl)-1,3-dioxolane (total: 5.73 g; 25 mmol) in TBP (20 mL) is added dropwise into the reaction mixture. The resulting solution is stirred at room temp for six hours, and then at 50-60° C. for 20 hours. After the mixture is cooled, a solution of 3-(trifluoromethyl)-benzaldehyde (4.35 g; 25 mmol) in TBF (20 mL) is added dropwise and the reaction is stirred at room temnp for 24 hours. The NH₄Cl saturated solution (50 mL) is added and stirred for 30 minutes. The organic layer is separated and the aqueous layer is extracted with ethyl acetate (2×100 mL). The organic layers are combined and dried over anhydrous Na₂SO₄. The solvent is removed and the residue is purified by column chromatography on silica gel. Eluting with ethyl acetate/hexanes (20:80) provide a colorless oil (3.23 g; 40%) as (3-(1,3-dioxolan-2-yl)phenyl)[3-(trifluoromethyl)phenyl]-methan-1-ol: MS 307.0 (M−17).

Procedure F

(Methyl-4-[(hydrazinylthioxomethyl) amino]benzoate: A mixture of methyl 4-isothiocyanato benzoate (193 mg; 1 mmol) and hydrazine (10 mg; 2 mmol) are stirred in toluene (7.5 mL) at RT for two hours. The solid is filtered, washed with a small volume of ethanol and hexanes and dried in vacuum. Yield: 192 mg (85%).

¹H NMR: (300 MHz, DMSO-d₆) 9.66δ (1H, s); 7.86δ (5H, m); 3.83δ (3H, s).

Procedure G

N-(3-bromophenyl)-2-[(3-nitrophenyl)carbonylamino]acetamide 3-nitrohippuric acid (250 mg; 1.116 mmol) is dissolved in methylene chloride (5 mL) containing a catalytic amount of DMAP, 1-ethyl-3-(3-dimethylaminopropyl)carbodiimide hydrochloride (640 mg; 3.34 mmol) and 3-bromoaniline (290 mg; 1.685 mmol). The solution is stirred for 18 hours at 25° C., diluted with enough methylene chloride to dissolve the resulting precipitate, and washed three times with 2N hydrochloric acid (aqueous) and saturated aqueous sodium chloride. The organic layer is dried with sodium sulfate, filtered, and stripped of solvent in vacuo. The resulting yellow solid is washed with 1:1 acetone/methylene chloride, then with 1:1 acetone/methanol to yield the title compound as a gray solid.

Procedure H

Iron powder (5.03 g; 56 mmol), water (5 mL) and hydrochloric acid (0.1 mL; 36 mmol) are added consecutively to a solution of methyl 2-[4-(3-nitrophenoxy)phenyl]acetate (1.3 g; 4.5 mmol) in ethanol (20 mL). After stirring at 95° C. for four hours, the solid is filtered while still hot and the filtrate is stripped of solvent in vacuo to yield methyl 2-[4-(3-aminophenoxy)phenyl]acetate as an oil. Yield: 1.1 g (95%).

¹H NMR: (300 MHz, DMSO-d₆) 7.24δ (2H, d); 6.96δ (3H, m); 6.31δ (1H, d); 6.13δ (1H, s); 6.10δ (1H, d); 5.23δ (2H, d); 3.65δ (2H, s); 3.62δ (3H, s).

Prophylaxis and Treatment of Disease

The compounds of the present invention inhibit tyrosine phosphatases, including PTP-1B, and thus improve insulin sensitivity, among other benefits. The compounds therefore will find use in preventing or treating Type 1 and Type 2 diabetes, improving glucose tolerance, improving insulin sensitivity when there is insulin resistance, lowering body weight, and preventing or treating obesity. In addition, the compounds will be useful in preventing or treating cancer, neurodegenerative diseases, and the like.

The present compounds may also be administered in combination with one or more further pharmacologically active substances e.g., selected from antiobesity agents, antidiabetics, antihypertensive agents, agents for the treatment and/or prevention of complications resulting from or associated with diabetes and agents for the treatment and/or prevention of complications and disorders resulting from or associated with obesity.

In a further aspect of the invention the present compounds may be administered in combination with one or more antiobesity agents or appetite regulating agents. Such agents may be selected from the group consisting of CART (cocaine amphetamine regulated transcript) agonists, NPY (neuropeptide Y) antagonists, MC4 (melanocortin 4) agonists, orexin antagonists, TNF (tumor necrosis factor) agonists, CRF (corticotropin releasing factor) agonists, CRF BP (corticotropin releasing factor binding protein) antagonists, urocortin agonists, B3 agonists, MSH (melanocyte-stimulating hormone) agonists, MCH (melanocyte-concentrating hormone) antagonists, CCK (cholecystokinin) agonists, serotonin re-uptake inhibitors, serotonin and noradrenaline re-uptake inhibitors, mixed serotonin and noradrenergic compounds, 5HT (serotonin) agonists, bombesin agonists, galanin antagonists, growth hormone, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, PPAR (peroxisome proliferator activated receptor) modulators, RXR (retinoid X receptor) modulators or TR B agonists.

In one embodiment of the invention the antiobesity agent is leptin. In other embodiments, the antiobesity agent is dexamphetamine or amphetamine, fenfluramine or dexfenfluramine, sibutramine, orlistat, mazindol or phentermine.

Suitable antidiabetics comprise insulin, GLP-1 (glucagons like peptide-1) derivatives such as those disclosed in WO 98/08871, which is incorporated herein by reference, as well as orally active hypoglycemic agents. The orally active hypoglycemic agents preferably comprise sulphonylureas, biguanides, meglitinides, oxadiazolidinediones, thizolidinediones, glucosidase inhibitors, glucagons antagonists such as those disclosed in WO 99/01423, GLP-1 agonists, potassium channel openers such as those disclosed in WO 98/26265 and WO 99/03861, insulin sensitizers, DPP-IV (dipeptidyl peptidase-IV) inhibitors, inhibitors of hepatic enzymes involved in stimulation of gluconeogensis and/or glycogenolysis, glucose uptake modulators, compounds modifying the lipid metabolism such as antihyperlipidemic agents and antilipedimic agents as HMG CoA inhibitors (statins), compounds lowering food intake, PPAR and RXR agonists and agents acting on the ATP-dependent potassium channel of the B-cells.

In one embodiment of the invention the present compounds are administered in combination with insulin. In further embodiments, the present compounds are administered in combination with a sulphonylurea e.g., tolbutamide, glibenclamide, glipizide or glicazide, a biguanide e.g. metformin, a meglitinide e.g., repaglinide, a thizolidinedione e.g., troglitazone, ciglitazone, pioglitazone, rosiglitazone or compounds disclosed in WO 97/41097 such as 5-[[4-[3-Methyl-4-oxo-3,4-dihydro-2-quinazolinyl]methoxy]phenyl-methyl]thiazolidine-2,4-dione or a pharmaceutically acceptable salt thereof, preferably the potassium salt.

Furthermore, the present compounds may be administered in combination with the insulin sensitizers disclosed in WO 99/19313 such as (−) 3-[4-[2-Phenoxazin-10-yl)ethoxy]phenyl]-2-ethoxypropanoic acid or a pharmaceutically acceptable salts thereof, preferably the arginine salt.

In further embodiments, the present compounds are administered in combination with an a-glucosidase inhibitor e.g. miglitol or acarbose, an agent acting on the ATP-dependent potassium channel of the B-cells e.g. tolbutamide, glibenclamide, glipizide, glicazide or repaglinide, nateglinide, an antihyperlipidemic agent or antilipidemic agent e.g., cholestyramine, colestipol, clofibrate, gemfibrozil, lovastatin, pravastatin, simvastatin, probucol or dextrothyroxine,

In still further embodiments, the present compounds are administered in combination with more than one of the above-mentioned compounds e.g., in combination with a sulphonylurea and metformin, a sulphonylurea and acarbose, repaglinide and metformin, insulin and a sulphonylurea, insulin and metformin, insulin, insulin and lovastatin, etc.

Furthermore, the present compounds may be administered in combination with one or more antihypertensive agents. Examples of antihypertensive agents are B-blockers such as alprenolol, atenolol, timolot, pindolol, propranolol and metoprolol, ACE (angiotensin converting enzyme) intubitors such as benazepril, captopril, analapril, fosinopril, lisinopril, quinapril and ramipril, calcium channel blockers such as nifedipine, felodipine, nicardipine, isradipine, rimodipine, diltiazem and verapamil, and a-blockers such as doxazosin, urapidil, prazosin and terazosin. Further reference can be made to Remington: The Science and Practice of Pharmacy, 19^{th} Edition, Gennaro, Ed., Mack Publishing Co., Easton, Pa., 1995.

It should be understood that any-suitable combination of the compounds according to the invention with one or more of the above-mentioned compounds and optionally one or more further pharmacologically active substances are considered to be within the scope of the present invention.

The therapeutically effective amounts of the present compounds will be a function of many variables, including the affinity of the inhibitor for the tyrosine phosphatase, any residual activity exhibited by competitive antagonists, the route of administration, the clinical condition of the patient, and whether the inhibitor is to be used for the prophylaxis or for the treatment of acute episodes.

In practicing the method of the present invention, the therapeutic preparation will be administered to a patient in need of treatment at a therapeutically effective dosage level. The lowest effective dosage levels can be determined experimentally by initiating treatment at higher dosage levels and reducing the dosage level until relief from reaction is no longer obtained. Generally, therapeutic dosage levels will range from about 0.01-100□g/kg of host body weight.

As discussed above, the present compounds can also administered in conjunction with other agents used in or proposed for the treatment of individual conditions as appropriate. However, when employed together with the present compounds, these agents may be employed in lesser dosages than when used alone.

Where combinations are contemplated, it is not intended that the present invention be limited by the particular nature of the combination. The present invention contemplates combinations as simple mixtures as well as chemical hybrids. One example of the latter is where the present compound is covalently linked to a pharmaceutical compound, or where two or more compounds are joined. For example, covalent binding of the distinct chemical moieties can be accomplished by any one of many commercially available cross-linking compounds.

In view of the therapeutic urgency attendant acute episodes, the present compounds may be intravenously infused or introduced immediately upon the development of symptoms. However, prophylaxis is suitably accomplished by intramuscular or subcutaneous administration. In this regard, the compositions are prepared as injectables, either as liquid solutions or suspensions; solid forms suitable for solution in, or suspension in, liquid prior to injection may also be prepared. These therapeutic preparations can be administered to mammals for veterinary use, such as with domestic animals, and clinical use in humans in a manner similar to other therapeutic agents. In general, the dosage required for therapeutic efficacy will vary according to the type of use and mode of administration, as well as the particularized requirements of individual hosts.

It is not intended that the present invention be limited by the particular nature of the therapeutic preparation. For example, such compositions can be provided together with physiologically tolerable liquid, gel or solid carriers, diluents, adjuvants and excipients. Such compositions are typically prepared as sprays (e.g. intranasal aerosols) for topical use. However, they may also be prepared either as liquid solutions or suspensions, or in solid forms including respirable and nonrespirable dry powders. Oral formulations (e.g. for gastrointestinal administration) usually include such normally employed additives such as binders, fillers, carriers, preservatives, stabilizing agents, emulsifiers, buffers and excipients as, for example, pharmaceutical grades of mannitol, lactose, starch, magnesium stearate, sodium saccharin, cellulose, magnesium carbonate, and the like. These compositions take the form of solutions, suspensions, tablets, pills, capsules, sustained release formulations, or powders, and typically contain 1%-95% of active ingredient, preferably 2%-70%.

The compounds of the present invention are often mixed with diluents or excipients that are physiologically tolerable and compatible. Suitable diluents and excipients are, for example, water, saline, dextrose, glycerol, or the like, and combinations thereof. In addition, if desired the compositions may contain minor amounts of auxiliary substances such as wetting or emulsifying agents, stabilizing or pH buffering agents.

Additional formulations which are suitable for other modes of administration, such as topical administration, include salves, tinctures, creams, lotions, and, in some cases, suppositories. For salves and creams, traditional binders, carriers and excipients may include, for example, polyalkylene glycols or triglycerides.

Determination of Activity

The compounds of the present invention are evaluated for biological activity as inhibitors of PTP-1B using, for example, a Malachite Green assay with pIRP as a substrate. The pIRP substrate includes a phosphotyrosine residue, and PTP-1B cleaves the phosphate group from the tyrosine, yielding the peptide and phosphate. The rate of the enzymatic reaction is determined by measuring the phosphate released during the reaction.

The reactants for the assay include 20 mM Tris-HCl, pH 7.4, 2 mM EDTA (ethylaminediamine tetraacetic acid) and 2 mM DTT (dithiothreitol) as the assay buffer, and 1 mM pIRP in assay buffer (1 mg in 0.59 mL buffer) as the substrate stock. The Malachite Green solution is prepared by adding 30 μL of 1% Tween 20 to 1 mL of Malachite Green Solution A. The stock of each compound to be tested is made up as 10 mM in DMSO (dimethylsulfoxide).

The compound to be tested is prepared as 1:5, 1:15.8, 1:50 and 1:158 dilutions from stock in a total volume of 100 μM DMSO. The reaction mixtures are prepared in a 96-well microtiter plate as 27.5 μL assay buffer, 3.5 μL of the diluted compound (to a final concentration of 100, 32, 10 and 3.2 μM), 10 μL of the pIRP substrate solution (to a final concentration of 200 μM) and 10 μL PTPase in assay buffer. The reactants are mixed well, the plate placed in a water bath at 30° C. and incubated for 3 minutes. The reaction is then terminated by adding 100 μL of Malachite Green solution per well, color is allowed to develop for 15 minutes, and the A₆₅₀ is measured by conventional means.

Unless otherwise indicated, this assay was used to determine activity for the selected compounds whose activity is recorded in the Table.

Alternatively, a pNPP assay can be used to screen compounds for tyrosine phosphatase inhibitory activity as follows: A 5× stock of pNPP (p-nitrophenol phosphate) substrate is prepared as 50 mM pNPP in assay buffer prepared as described above. Various tyrosine phosphatase solutions can be prepared as follows:
PTP-1B (SBI purified, 1 mg/mL) as a 1:250 dilution (to a final concentration of 4 μg/mL);
TC-PTP (NEB, 1000 units in 100 μL) as a 1:50 dilution (to a final concentration of 2U/10 μL (4 μg/mL));
CD45 (Calbiochem, 20 μg, 400 units in 100 μL) as a 1:50 dilution (to a final concentration of 0.8U/10 μL (4 μg/mL));
LAR (NEB, 1000 units in 200 μL) as a 1:75 dilution (to a final concentration of 0.7U/10 μL (4 μg/mL)); and
PTP-β (UBI, #14-350, 10,000 units, 40 μg/571 μL) as a 1:17.5 dilution (to a final concentration of 10U/10 μL (4 μg/mL));

The compound to be tested is prepared as 1:16.7 and 1:50 dilutions from stock in a total volume of 100 μM DMSO to give final concentrations of 626 and 200 μM. The reaction mixtures are prepared in a 96-well microtiter plate (on ice) as 55 μL assay buffer, 5 μL of the diluted compound (to a final concentration of 31.3 and 10 μM), 20 μL of the pNPP substrate solution (to a final concentration of 10 mM) and 20 μL PTease in assay buffer. The reactants are mixed well, the plate placed in a water bath at 30° C. and incubated for 10 minutes. The reaction is then terminated by adding 100 μL of 2M K₂CO₃ per well, and the absorbance is measured at 405 nm by conventional means.

Compounds which demonstrate inhibitory activity against tyrosine phosphatases can have application in the treatment of various diseases. For example, compounds which demonstrate inhibitory activity against PTP-1B can find use in the treatment of diabetes. Compounds which demonstrate such activity against CD45 can find use in the treatment of autoimmune diseases, inflammation, transplantation rejection reactions, and other diseases including arthritis, systemic lupus, Crohn's disease, inflammatory bowel disease, and other autoimmune disorders known to those skilled in the art. Compounds which demonstrate such activity against TC-PTP can find use in the treatment of cancer, typically as antiangiogenic agents.

In the case of compounds which demonstrate inhibitory activity against PTP-1B, one can test the compounds for blood glucose lowering effects in diabetic obese female ob/ob mice as follows: The mice will be of similar age and body weights and randomized into groups of ten mice. They have free access to food and water during the experiment.

The compounds are administered by either gavage, subcutaneous, intravenous or intraperitoneal injections. Examples of typical dose ranges for such evaluations are 0.1, 0.3, 1.0, 3.0, 10, 30, 100 mg per kg body weight. The blood glucose levels are measured twice before administration of the compounds of the invention. After administration of the compound, the blood glucose levels are measured at the following time points: 1, 2, 4, 6, and 8 hours. A positive response is defined either as (i) a more than 25 percent reduction in blood glucose levels in the group receiving the compound of the invention compared to the group receiving the vehicle at any time point or (ii) statistically significant (i.e., p<0.05) reduction in the area under the blood glucose curve during the whole period (i.e. 8 hrs) in the group treated with the compounds of the invention compared to controls. Compounds that show positive response can be used as development candidates for treatment of human diseases such as diabetes and obesity.

The invention now being generally described, the same will be better understood by reference to the following detailed examples, which are provided for illustration and are not to be considered as limiting the invention unless so specified. The structures of various of the disclosed compounds will be found depicted in FIG. 1.

### EXPERIMENTAL

In the experimental disclosure which follows, all weights are given in grams (g), milligrams (mg), micrograms (μg), nanograms (ng), or picograms (pg), all amounts are given in moles (mol), millimoles (mmol), micromoles (pmol), nanomoles (umol), picomoles (pmol), or femtomoles (fmol), all concentrations are given as percent by volume (%), proportion by volume (v:v), molar (M), millimolar (mM), micromolar (μM), nanomolar (nM), picomolar (pM), femtomolar (S), or normal (N), all volumes are given in liters (L), milliliters (mL), or microliters (μL), and linear measurements are given in millimeters (mm), micrometers (μm), or nanometers (nm) and mp is melting point, unless otherwise indicated.

### Example 1

N2-(3-bromophenyl)-5-(3-nitrophenyl)-1,3,4-thiadiazol-2-amine

To a solution of 3-bromophenyl isothiocyanate (6.5 g; 30.4 mmol) in toluene (150 mL) is added 3-nitrobenzhydrazide (5.0 g; 27.6 mmol) under argon. The reaction mixture is heated at reflux for two hours. The mixture is filtered while the toluene still is warm. The solid is washed with warm toluene (3×50 mL) and dried to yield N1-(3-bromophenyl)-2-(3-nitrobenzoyl) hydrazine-1-carbothioamide (10.7 g; 98%) (mp. 163-165° C.). The product is used for the next step without further purification.

To a slurry mixture of the above carbothioamide (5.0 g; 12.7 mmol) in toluene (5 mL) at 0° C. is dropped con. H₂SO₄ (2.5 mL). The reaction mixture is stirred at room temperature (RT) for three hours. The toluene is removed and ice-H₂O (50 mL) is added. The mixture is neutralized with NH₃.H₂O until pH 8 and filtered. The solid product is recrystallized with DMSO/MeOHEH₂O (3:5:10) to yield N2-(3-bromophenyl)-5-(3-nitrophenyl)-1,3,4-thiadiazol-2-amine (4.5 g; 94%). mp. 273-275° C. mass spec obsd M⁺376.88, M⁺-NO₂ 330.93, calcd exact mass 377.22 (C₁₄H₉BrN₄O₂S).

¹H NMR (DMSO-d₆) δ 7.23 (d, 1H), 7.34 (t, 1H), 7.52 (d, 1H), 7.82 (t, 1H), 8.10 (s, 1H), 8.32 (t, 2H), 8.62 (s, 1H).

¹³C NMR (DMSO-d₆) δ 116.5, 119.9, 120.7, 122.0, 124.6, 124.8, 131.0, 131.5, 133.1, 141.7, 148.3, 156.1.

### Example 2

Methyl (3-(3-{5-[(3,4-dichlorophenyl)amino]-1,3,4-thiadiazol-2-yl}phenoxy)benzoate

A solution of the product from Procedure B (methyl 3-(3-carbonylphenoxy)benzoate) (750 mg; 2.9 mmol) and (aminoamino)[(3,4-dichlorophenyl)amino]methane-1-thione (692 mg; 2.9 mmol) in dry ethanol (8 mL) under argon is refluxed for two hours. After cooling to RT, the mixture was filtered and the solid washed by ethanol. The solid was suspension in dry ethanol (5 mL) and iron (III) chloride hexahydrate (1.546 g; 5.7 mmol) was added. The reaction mixture was refluxed for two hours, then cooled to RT. The solid was collected by filter and washed by ethanol, then crystallized from ethyl acetate/hexanes to yield the title compound (348 mg; 25%) of as a yellow solid.

¹H NMR (300 MHz, d₆-DMSO): δ 10.92 (1H, s), 8.13 (1H, d, J=2.1 Hz), 7.79 (1H, d, J=7.5 Hz), 7.68-7.55 (5H, m), 7.49 (1H, dd, J=8.7, 2.1 Hz), 7.43 (1H, dd, J=8.1, 2.4 Hz), 7.21 (1H, d, J=8.1 Hz), 3.84 (3H, s).

### Example 3

(3,4-dichlorophenyl)[5-(3,5-dinitrophenyl)(1,3,4-thiadiazol-2-yl)]amine

To the product from Procedure D (59 mg; 0.25 mmol) was added 3,5-dinitrobenzoyl chloride (50 mg; 0.25 mmol) in dichloromethane (˜1 mL) at 0° C. (under argon). The reaction was allowed to rise to ambient temperature (˜25° C.) and stirred for one hour. The solvent was removed by vacuum and the residue was subjected to silica gel flash chromatography (3% methanol in dichloromethane). The resulting intermediate was dissolved in toluene (˜1.5 mL) with con. H₂SO₄ (˜1 eq) and refluxed for two hours. Water and ammonium hydroxide were added (to neutralize) and the precipitate was collected by vacuum filtration. Recrystallization (ethanol/water) yielded the title compound (61% yield). mp 305-306.5° C.

### Example 4

(4-{2[(3,4-dichlorophenyl)amino](1,3-thiazolfyl)}phenyl)diethylamine

To 3,4 dichlorobenzeneisothiocyanate (1 g; 4.8 mmol) in dry dioxane (15 mL) at RT was added ammonium hydroxide (0.8 mL; 0.023 mmol) and ammonium acetate (1.5 g; 19.5 mmol). The reaction mixture was refluxed for one hour. The solvent was removed under reduced pressure which afforded amino[(3,4-dichlorophenyl)amino]methane-1-thione (1.04 g; 98%) as white solid.

¹H NMR (CDCl₃) 9.88 (s, 1H); 7.91(s, 1H); 7.56 (d, 1H); 7.39 (d, 1H).

To a solution of amino[(3,4-dichlorophenyl)amino]methane-1-thione (100 mg; 4.32 mmol) in dioxane (3 mL) at RT was added alpha-bromo-4-(diethylamino)acetophenone (121 mg; 4.5 mmol). The reaction mixture was stirred at RT for one hour and pyridine (0.4 mL; 4.66 mmol) was added. After refluxing for five hours, the reaction mixture was concentrated under reduced pressure, quenched with water, and the resulting mixture was extracted with ethyl acetate. The organic layer was washed with water, then with brine, and dried over anhydrous sodium sulfate. Concentration under reduced pressure afforded a crude product which was purified by flash chromatography (50/50 Ethyl acetate/Hexane) to yield the title compound (128 mg; 75%) as a purple solid. The product had a HPLC retention time of 3.27 min (Column: C18, 5 μm, 50×3 mm); Solvent A=(H₂O, 10% AcOH); solvent B=(Acetonitrile, 10% AcOH) and LC/SM M+=393.

¹H NMR (CDCl₃) 7.70-7.67(m, 2H); 7.37-7.26(m, 3H); 6.72-6.62(m, 3H); 3.42-3.35(m, 4H); 1.21-1.16(m, 3H)

### Example 5

(3-Bromophenyl)-[5-(3-nitrophenyl)-1,3,4-thiadiazol-2-yl]-amine

A mixture of 3-nitrophenacylamine hydrochloride (620 mg; 2.8 mmol) and sodium bicarbonate (240 mg; 2.8 mmol) in H₂O (10 mL) was added to a solution of 3-bromophenylisothiocyanate (580 mg; 2.7 mmol) in acetone (23 mL). The resulting homogeneous solution was stirred at RT for 30 minutes, after which it was partitioned between ethyl acetate and water. The ethyl acetate layer was dried over Na₂SO₄ and evaporated to dryness to yield N-(3-nitrophenacyl)-N′-(3-bromophenyl)-thiourea (400 mg; 38%). The thiourea was suspended in acetic anhydride (10 mL) containing polyphosphoric acid (0.5 mL). After stirring for 12 hours at RT, the mixture was poured over ice. The resulting solids were isolated via filtration and triturated with 4/1 ethyl acetate/hexanes to yield pure title compound as a yellow solid (150 mg; 38%); mp 228-231° C., MS m/z 377.65 [MH⁺].

¹H NMR (300 MHz, DMSO-d₆) δ 7.17 (d, J=7.8 Hz, 1H), 7.30 (t, J=8.1 Hz, 1H), 7.52 (d, J=7.8 Hz, 1H), 7.69 (t, J=7.8 Hz, 1H), 7.99 (d, J=8.4 Hz, 2H), 8.09 (d, J=6.0 Hz, 2H), 8.31 (s, 1H), 10.72 (br s, 1H).

### Example 6

(3-bromophenyl)[2-(3-nitrophenyl)(1,3-thiazol-5-yl)]amine

Without further purification, the intermediate from Procedure G (20 mg; 0.053 mmol) was combined with 2,4-bis(4-methoxyphenyl)-1,3,2,4-dithiadiphosphetane-2,4-dithione (Lawesson's reagent, 100 mg; 0.1 mmol) in pyridine (3 mL) and heated to reflux for three hours. The reaction mixture was diluted with ethyl acetate, extracted 3× with 2N HCl, 1× with saturated aqueous potassium carbonate, and 1× with saturated aqueous sodium chloride. The organic layer was dried with sodium sulfate, filtered, and stripped of solvent in vacuo. The resulting solid was purified using silica chromatography using methylene chloride and 97:3 methylene chloride/methanol as solvents, yielding the title compound as an orange solid (12 mg; 60% yield). Mass Spectrum (electrospray, positive ion): calculated for MH+377, found 377; calculated for MNa+400, found 400.

### Example 7

(3-(3-{5[(3,4-dichlorophenyl)amino]-1,3,4-thiadiazol-2-yl}phenoxy)benzoic acid)

The product from Example 2 (methyl 3-(3-{5-[3,4-dichlorophenyl)amino]-1,3,4-thiadiazol-2-yl}phenoxy)benzoate (228 mg; 0.48 mmol) and LiOH (32 mg; 1.3 mmol) were suspension in THF/MeOH/H₂O (2/3/9) (30 mL). The mixture was stirred overnight at RT and concentrated in vacuo. The residue was washed by ethyl acetate, then 15% HCl aqueous solution was added until the pH was <7. The solid was collected by filter and washed by water, then crystallized from ethanol/ethyl acetate/hexanes yields 3-(3-{5-[(3,4-dichlorophenyl)amino]-1,3,4-thiadiazol-2-yl}phenoxy)benzoic acid (195 mg; 89%) as a white solid. mp 258-259° C.; MS (M+H)⁺458.2.

¹H NMR (300 MHz, d₆-DMSO): δ 13.21 (1H, br s), 10.94 (1H, s), 8.13 (1H, d, J=2.4 Hz), 7.70 (1H, d, J=8.4 Hz), 7.68-7.49 (7H, m), 7.40 (1H, dd, J=7.8, 2.1 Hz), 7.21 (1H, dd, J=8.1, 1.5 Hz).

### Example 8

(3,4-dichlorophenyl){[4-(tert-butyl)phenyl]methyl}[5-(3-ethoxyphenyl)(1,3,4-thiadiazol-2-yl)]amine and

(1-(2-[aza(3,4-dichlorophenyl)methylenel-3-{[4(tert-butyl)phenyl]methyl}(1,3,4-thiadiazolin-5-yl)-3-ethoxybenzene)

To a solution of (3,4-dichlorophenyl)[5-(3-ethoxyphenyl)(1,3,4-thiadiazol-2-yl)]amine (120 mg; 0.33 mmol) in dry DMF (5 mL) was added a solution of potassium tert-butoxide (0.33 mL; 1M; 0.33 mmol) in THF at RT under an argon atmosphere. After five minutes, 4-(tert-butyl)benzyl bromide (89 mg; 0.39 mmol) was injected, and the solution was stirred overnight. The DMF was evaporated (rotavap) under vacuum. The crude residue was purified by flash chromatography (ethyl acetate/hexanes, 100% hexanes to 1:4) to the isolation of the less polar isomer (1-(2-[aza(3,4-dichlorophenyl)methylene]-3-{[4-(tert-butyl)phenyl]methyl}(1,3,4-thiadiazolin-5-yl))-3-ethoxybenzene) (20 mg) in 12% yield R_{f}=0.60 (ethyl acetate/hexanes, 1:2).

¹H NMR (300 MHz, d₁-CDCl₃): δ 7.42-6.92 (11H, m), 5.29 (2H, s), 4.05 (2H, q), 1.42 (3H, t), 1.32 (9H, s).

The other, more polar isomer, (3,4-dichlorophenyl){[4-(tert-butyl)phenyl]methyl}[5-(3-ethoxyphenyl)(1,3,4-thiadazol-2-yl)]amine (122 mg; 72%) was also isolated. R_{f}=0.48 (ethyl acetate/hexanes, 1:2);

¹H NMR (300 MHz, d₁-CDCl₃): δ 7.49-6.92 (11H, m), 5.18 (2H, s), 4.07 (2H, q), 1.43 (3H, t), 1.30 (9H, s).

### Example 9

3-[3-(3-{5-[(3,4-Dichlorophenyl)amino]-1,3,4-thiadiazol-2-yl}phenoxy)phenyl]propanoic acid

Trimethlsilyl diazomethane (15 mL; 30 mmol) was added to an ice cold solution of 3-(3-hydroxyphenyl)propanoic acid (3.32 g; 20 mmol) in acetonitrile (40 mL). The mixture was stirred cold for 30 minutes and slowly brought to RT and continued to stir overnight. Acetic acid (1 mL) was added to quench the excess trimethylsilyl diazomethane. The reaction mixture was diluted with methanol (10 mL) and 1M hydrochloric acid (2 mL), followed by rotoevaparation of the solvents. The residue was purified over silica gel column. The compound was eluted with EtOAC/Hexanes (1/1) to yield methyl 3-(3-hydroxyphenyl)propanoate as an oil.

Yield: 3.2 g (90%). TLC (silica gel): R_{f}=0.8 (EtOAc/Hexane=1/1).

Methyl 3-[3-(3-formylphenoxy)phenyl]propanoate was prepared using Procedure B from methyl 3-(3-methoxyphenyl)propanoate (2.7 g; 15 mmol), 3-bromobenzaldehyde (1.75 mL; 15 mmol), copper oxide (2.4 g; 30 mmol) and potassium carbonate (4.14 g; 30 mmol) in pyridine as an oil. Yield: 1.7 g (40%).

¹H NMR: (300 MHz, CDCl₃) 9.96δ (1H, s); 7.60δ (1H, d); 7.50δ (1H, t); 7.45δ (1H, s); 7.25δ (2H, t); 7.01δ (1H, d) 6.88δ (1H, s); 6.86δ (1H, d); 3.66δ (3H, s); 2.95δ (2H, t); 2.63δ (2H, t).

Methyl 3-(3-{3-[(1E)-2-aza-2-({[(3,4-dichlorophenyl)amino]thioxomethyl}amino) vinyl]phenoxy}phenyl)propanoate was prepared using the procedure in Example 2 from methyl 3-[3-(3-formylphenoxy)phenyl]propanoate (426 mg; 1.5 mmol) and the product from Procedure D (354 mg; 1.5 mmol) to obtain a white solid. Yield: 450 mg (60%). Mass: M⁺: 502 (Calc.); 502 (Obsd.).

Ethyl 3-[3-(3-{5-[(3,4-dichlorophenyl)amino]-1,3,4-thiadiazol-2-yl}phenoxy) phenyl]propanoate was prepared using the procedure in Example 2 from methyl 3-(3-{3-[(1E)-2-aza-2-({[(3,4-dichlorophenyl)amino]thioxomethyl}amino)vinyl]phenoxy}phenyl)propanoate (400 mg; 0.8 mmol) and iron(III) chloride hexahydrate (432 mg; 1.6 mmol). This product was used in the next reaction without further purification. Yield: 250 mg (61%). Mass: M⁺: 514 (Calc.); 514 (Obsd).

3-[3-(3-{5-[(3,4-Dichlorophenyl)amino]-1,3,4-thiadiazol-2-yl}phenoxy) phenyl]propanoic acid: The title compound was prepared using the procedure for Example 7 from ethyl 3-[3-(3-{5-[(3,4-dichlorophenyl)amino]-1,3,4-thiadiazol-2-yl}phenoxy)phenyl]propanoate (150 mg; 0.29 mmol) and lithium hydroxide (24 mg; 1 mmol). Yield: 130 mg (91%). mp 194-196° C. Mass: M⁺: 486 (Calc.); 486 (Obsd.).

### Example 10

2-[4-(Phenylmethoxy)phenyl]-N-(5-{3-[3-(trifluoromethyl)phenoxy]phenyl}(1,3,4-thiadiazol-2-yl))acetamide

A mixture of the product from Example 23 (202 mg; 0.6 mmol) and 2-[4-(phenylmethoxy)phenyl]acetyl chloride (313 mg; 1.2 mmol), and dimethyl-4-pyridylamine (244 mg; 2.0 mmol) in CH₂Cl₂ (3 mL) was stirred at RT for 20 hours, then 1N HCl aqueous (10 mL) was added and stirred at RT for one hour. The aqueous layer was removed and the CH₂Cl₂ was concentrated. Water was added and filtration provided the title compound (107 mg; 32%), which was washed with water and then ethyl acetate: mp 194-195° C.; Mass (M)⁺562 (Calc.); 562 (Obsd.).

### Example 11

(3-bromophenyl)[5(3-{[3-trifluoromethyl)phenyl]methoxy]phenyl)(1,3,4-thiadiazol-2-yl)]amine

A mixture of 3-{5-[(3-bromophenyl)amino]-1,3,4-thiadiazol-2-yl}phenol (100 mg; 0.29 mmol) and 3-(bromomethyl)-1-(trifluoromethyl)benzene (82 mg; 0.34 mmol) in dry DMF (5 mL) were stirred under argon at RT. Cesium carbonate (56 mg; 0.17 mmol) was added and this mixture stirred overnight. The reaction mixture was heated at 50° C. for one hour. The DMF was evaporated (rotovap) under vacuum. The crude residue was purified by flash chromatography (ethyl acetate/hexanes, 1:10 to 1:2) to yield the title compound ((3-bromophenyl)[5-(3-{[3-(trifluoromethyl)phenyl]methoxy}phenyl)(1,3,4-thiadiazol-2-yl)]amine) (92 mg; 63%) as white solid. mp 186-187° C. NMR: GE 300, QE+, LCMS: Finnigan, SSQ 7000 Mass Spectrometer

### Example 12

3-[(3-{5[(3,4-Dichlorophenyl)amino](1,3,4-thiadiazol-2-yl)}phenyl)[3-(trifluoromethyl)phenyl]methylthio]propanoic acid

A mixture of 3-[(3-{5-[(3,4-dichlorophenyl)amino](1,3,4-thiadiazol-2-yl)}phenyl)[3-(trifluoromethyl)phenyl]methan-1-ol (248 mg; 0.5 mmol) and 3-mercaptopropionic acid (531 mg; 5.0 mmol) in trifluoroacetic acid (1 mL) was stirred at RT for 20 hours. The solvent and excess 3-mercaptopropionic acid was removed by rotovapor. The residue was purified by column chromatography on silica. Eluting with ethyl acetate provided the title compound as a slightly brown solid (40 mg; 16%): mp 140-141° C.; MS 583.9 (M).

### Example 13

(3,4-dichlorophenyl)[5-(3-{ethoxy[3-trifluoromethyl)phenyl]methyl}phenyl)(1,3,4-tridiazole-2-y10]amine

Phosphorus tribromide (0.300 mL; 6.66 mmol) was added dropwise to a solution of ((3-(1,3-dioxan-2-yl)phenyl)[3-trifluoromethyl)phenyl]methan-1-ol) (2.15 g; 6.66 mmol) in ether (20 mL) while stirring under nitrogen at 0° C. After stirring for 2.5 hours at RT, the reaction was quenched by slow addition of the reaction mixture onto ice (100 g). Following separation of layers, the aqueous layer was extracted with ether (3×75 mL). The organic layer was dried with magnesium sulfate, and evaporated under reduced pressure. The crude was purified using flash chromatography to yield (3-{bromo[trifluoromethyl)phenyl]methyl}phenyl)formaldehyde (1.42 g; 4.15 mmol; 63%) as a colorless oil.

¹H NMR: 300 MHz; CDCl₃; 10.02 (s, 1H); 8.14-7.26(m, 8H); 6.32(s, 1H).

A solution of this aldehyde (290 mg; 0.85 mmol) and the product from Procedure D (200 mg; 0.85 mmol), in ethanol (4 mL) was refluxed for 1.5 hours, cooled to RT, and Iron (II) chloride hexahydrate was added to the reaction mixture. The reaction mixture was heated under reflux for two hours and then was cooled to RT. The solid was filtered and dried to yield the title compound (150 mg; 0.29 mmol). mp 200-205° C.; mass spectrum M+=525.

¹H NMR: 300 MHz; CDCl₃; 7.93-7.26 (m, 12H); 5.46(s, 1H); 3.58-3.55(m, 2H); 1.33-1.28(m, 3H).

### Example 14

{5-[3-(3-(1H-1,2,3,4-tetraazol-5-yl)phenoxy)phenyl](1,3,4-thiadiazol-2-yl)}(3,4-dichlorophenyl)amine

To the product from Example 19 (120 mg; 0.28 mmol) in DMF (1.5 mL), under argon, was added ammonium chloride (42 mg; 0.82 mmol) and sodium azide (53 mg; 0.82 mmol). The reaction was then heated at 115° C. for 96 hours. TLC analysis revealed that a new product had formed in addition to unreacted starting material. The mixture was cooled to RT, diluted with water, extracted with dichloromethane, and dried over magnesium sulfate. The crude oil was subjected to silica chromatography (100% dichloromethane; 5% methanol in dichloromethane) to yield pure tetrazole (45 mg), compound 2. Isolated yield 31%, uncorrected mp 227 to 229° C. (with decomposition).

### Example 15

Ethyl 2-[(3-{5-[(3,4-Dichlorophenyo)amino](1,3,4-thiadiazol-2-yl)}phenyl)[3-(trifluoromethyl)phenyl]methoxyl]acetate

A solution of (3-(1,3-dioxolan-2-yl)phenyl)[3-(trifluoromethyl)phenyl]methan-1-ol (3.23 g; 10 mmol) and sodium hydrade (260 mg; 11 mmol) in TBF (20 mL) was stirred under Argon at room temp for 1 hours. A solution of tert-butyl bromoacetate (1.95 g; 10 mmol) in TBF (10 mL) was added and the resulting solution was refluxed for 24 hours. Water (10 mL) was added to quench the reaction. The organic layer was separated and the aqueous layer was extracted with ethyl acetate (2×100 mL). The organic layers were combined and dried over anhydrous Na₂SO₄. The solvent was removed and the residue was purified by column chromatography on silica gel. Eluting with ethyl acetate/hexanes (20:80) provide a colorless sticky oil (2.4 g) as a mixture of tert-butyl 2-{(3-(1,3-dioxolan-2-yl)phenyl)[3-(trifluoromethyl)phenyl]methoxy} acetate and (3-(1,3-dioxolan-2-yl)phenyl)[3-(trifluoromethyl)phenyl]methan-1-ol.

A sample of the product from the preceding paragraph (2.45 g) was combined, without further purification, with pyridinium tosylate (500 mg; 2.0 mmol) in acetone/water (4:1; 2 mL). The resulting solution was refluxed for two days. The solvent was removed and the residue was extracted with ethyl acetate (3×30 mL). The organic layers were combined and dried over anhydrous Na₂SO₄. The solvent was removed and the residue was purified by flash column chromatography on silica gel. Eluting with ethyl acetate/hexanes (20:80) provided a sticky oil (1.53 g) as a mixture of tert-butyl 2-{(3-carbonylphenyl)[3-(trifluromethyl)phenyl]methoxy} acetate and (3-{hydroxy[3-(trifluromethyl)phenyl]methyl}phenyl)formaldehyde.

A sample of the product from the preceding paragraph (1.52 g) was combined, without further purification, with the product from Procedure D (1.28 g; 5.4 mmol) in ethanol (20 mL). The resulting solution was refluxed for five hours. After cooling, the solution was concentrated and the residue was purified by flash column chromatography on silica gel. Eluting with ethyl acetate provided a brown solid (2.51 g) as a mixture of tert-butyl 2-({3-[2-aza-2-({[(3,4-dichlorophenyl)amino]thioxomethyl}-amino)vinyl]phenyl}[3-(trifluoromethyl)phenyl]methoxy)acetate and {3-[2-aza-2-({[(3,4-dichlorophenyl)amino]-thioxomethyl}amino)vinyl]phenyl}[3-(tifluoromethyl)phenyl]methan-1-ol.

A sample of the product from the preceding paragraph (2.49 g) was combined, without further purification, with iron (III) chloride hexahydrate (4.05 g; 15 mmol) in ethanol (20 mL). The resulting solution was refluxed for five hours. After cooling, the solution was concentrated and water (20 mL) was added. A brown sticky solid was filtered. This crude product was purified by flash column chromatography on silica gel. Eluting with ethyl acetate/hexanes (1:1) provided a brown solid (1.95 g), which is a mixture of 3-[(3-{5-[(3,4-dichlorophenyl)amino](1,3,4-thiadiazol-2-yl)}phenyl)[3-(trifluoromethyl)-phenyl]methan-1-ol and ethyl 2-[(3-{5-[(3,4-dichlorophenyl)amino](1,3,4-thiadiazol-2-yl)}phenyl)[3-(trifluoromethyl)phenyl]methoxyl]acetate. This mixture (800 mg) was separated further by column chromatography on silica gel. Eluting with ethyl acetate/hexanes (20:80) provided ethyl 2-[(3-{5-[(3,4-dichlorophenyl)amino](1,3,4-thiadiazol-2-yl)}phenyl)[3-(trifluoromethyl)phenyl]methoxyl]acetate (200 mg): mp 135-136° C.; MS 582.0 (M); and also provided 3-[(3-{5-[(3,4-dichlorophenyl)amino]-(1,3,4-thiadiazol-2-yl)}phenyl)[3-(trifluoromethyl)phenyl]methan-1-ol (250 mg): mp 191-192° C.; MS 495.8 (M).

### Example 16

(3-methoxy-1-(6-(4-phenylphenyl)imidazolo(2,1-b)1,3,4-thiadiazolin-2-yl)benzene)

A mixture of anisic acid (9.88 g; 65 mmol), thiosemicarbozide (5.92 g; 65 mmol) and phosphorous oxychloride (32.08 g; 210 mmol) was refluxed gently for 30 minutes. After cooling in an ice bath, a mixture of ice and water (60 mL) was added with stirring. The mixture was slowly warmed and refluxed for four hours. The reaction was cooled, neutralized with 5M potassium hydroxide solution and the solid was filtered. The solid was washed with water, then ether and dried to obtain 5-(3-methoxyphenyl)-1,3,4-thiadiazole-2-ylamine. Yield: 8.10 g (60%).

¹H NMR: (300 MHz, DMSO-d6) 8.37δ (2H, b), 7.41δ (1H, t), 7.39δ (2H, m), 7.07δ (1H, d), 3.82δ (3H, s).

A solution of 5-(3-methoxyphenyl)-1,3,4-thiadiazole-2-ylamine (1.8 g; 8.7 mmol) and 2-bromo-4′-phenyl acetophenone (2.4 g; 8.7 mmol) were refluxed for about 36 hours. The solid was filtered, washed with ethanol and hexane and dried to yield the title compound. Yield: 2.3 g (69%).

¹H NMR: (300 MHz, DMSO-d6) 8.84δ (1H, s), 7.99δ (2H, d), 7.74δ (4H, m), 7.51δ (5H, m), 7.39δ (1H, d), 7.22δ (1H, m), 3.88δ (3H, s). Mass: MH+: 384 (Calc.); 384 (Obsd.).

### Example 17

N-{(1E)-2-aza-2-(3-bromophenyl)-1-[(4-phenylphenyl)methylthiolvinyl}[3-(trifluoromethyl)phenylcarboxamide

A solution of amino[(3-Bromophenyl)amino]methane-1-thione (100 mg; 0.43 mmol) and 4-(bromomethyl-1-phenylbenzene (106 mg; 0.43 mmol) in dioxane (4 mL) was refluxed for four hours. Most of the solvent was evaporated under vacuum, and the residue was washed with ether (3×20 mL) to provide {[(3-Bromophenyl)amino][(4-phenylphenyl)methylthio]methyleneamine hydrobromide (160 mg; 78%) as a yellow solid. The intermediate was 95% pure by LC-MS (SSQ 7000) (retention time=3.26 min.; M+=398 free base. Column: Betasil C18 5 μm 50×3 mm.; Solvent A=water-0.1% AcOH; B=Acetonitrile −0.1% AcOH).

To a solution of {[(3-Bromophenyl)amino][(4-phenylphenyl)methylthio]methyleneamine hydrobromide (100 mg; 0.2 mmol) in dry pyridine (5 mL) was added 3-(trifluoromethyl)benzoyl chloride at ambient temperature. The resulting solution was refluxed for 15 minutes. The cold reaction mixture is poured into the water (10 mL). After filtration, the residue was washed with water and tecrystallized from ethanol to yield the title compound. The product was 95% pure by LC-MS (SSQ 7000) (retention time=4.33 min.; M+=570 free base. Column: Betasil C18 5 μm 50×3 mm.; Solvent A=water-0.1% AcOH; B=Acetonitrile-0.1% AcOH). mp 220-225° C. from ethanol.

### Example 18

[3-(3-{5-[(3,4-dichlorophenyl)amino](1,3,4-thiadiazol-2-yl))phenoxy)phenyl]-N-1,3-dioxolan-2-ylmethyl)carboxamide

To 3-(3-{5-[(3,4-dichlorophenyl)amino]-1,3,4-thiadiazol-2-yl}phenoxy)benzoic acid (50 mg; 0.11 mmol) in DMF (1.5 mL) was added 1,3-dioxolan-2-ylmethylamine (10 μL; 0.11 mmol). The mixture was warmed to dissolve the reactants and HATU (45 mg; 0.12 mmol) followed by triethylamine (49 μL) were added and the mixture was stirred for four hours. The solution was diluted with 0.5N aqueous HCl and extracted with dichloromethane (3×3 mL). The solvent was removed by high vacuum and the resulting residue was purified by silica gel chromatography (9/1 Hex/EtOAc; 7/3 Hex/Oac). Yield: 27%, 15 mg., mp 200-203° C.

### Example 19

3-(3-{5[3,4-dichlorophenylaminol-1,3,4-thiadiazol-2-yl}phenoxy)benzeneearbonitrile

3-hydroxybenzaldehyde and 3-bromobenzenecarbonitrile were combined as in Procedure B to yield 3-(3-carbonylphenoxy)benzenecarbonitrile. Yield: 12% (isolated)

¹H NMR (300 MHz, CDCl₃): δ 10.00 (1H, s); δ 7.71 (1H, d, J=7.5 Hz); δ 7.58 (1H, t, J=7.8 Hz); δ 7.50-7.42 (3H, m); δ 7.33-7.26 (3H, m).

3-(3-Formyl-phenoxy)-benzonitrile, 67 mmol, and [(3,4-dichloropheny)amino]hydrazinomethane-1-thione, 67 mmol, were combined in ethanol and heated to reflux for two hours. The solution was cooled to room temperature and the precipitate was collected by filtration, and then washed with hexane (3×). The white solid was slurried in ethanol and 3 eq of Fe(III)Cl₃ was added. The slurry was refluxed for two hours, cooled and the crude product was collected by filtration. The crude product was washed (3×) with water followed by hexane. The product was recrystallized from hot ethanol to give 1.4 g of the title compound. Yield 47%. mp 237-239° C. MS m/z 437.5 [M−H].

### Example 20

3-{5-[(3,4-dichlorophenyl)amino](1,3,4-thiazol-2-yl)}3-(trifluoromethyl)phenyl]methan-1-ol

The product from Procedure E was refluxed for 18 hours with pyridinium p-toluenesulfonate in 4:1 acetone/water. After removal of acetone in vacuo, water and ethyl acetate were added. The organic layer was washed 3 times with water, then with saturated sodium chloride, dried over sodium sulfate, filtered and stripped of solvent in vacuo to yield (3-{hydroxy[3-(trifluoromethyl)phenyl]methyl}phenyl)formaldehyde.

A solution of [(3,4-dichlorophenyl)amino]hydrzinomethane-1-thione (200 mg; 0.85 mmol), and (3-{hydroxy[3-(trifluoromethyl)phenyl]methyl}phenyl)formaldehyde (290 mg; 0.85 mmol) in ethanol (4 mL) was refluxed for 1.5 hours, cooled to RT, and Iron (III) chloride hexahydrate was added to the reaction mixture. And the reaction mixture was heated under reflux for two hours. The reaction was cooled to RT, and the ethanol was removed under reduced pressure. The crude was purified by flash chromatography to yield the title compound (25 mg; 0.05 mmol, 16%)

### Example 21

(2E)-3-{4[5-((3-[(2-Phenylphenyl)methylthio]phenylamino)(1,3,4-thiadiazol-2 yl)]phenyl}prop-2-enoic acid

(2E)-3-[4-((1E)-2-Aza-2-{[({3-[(2-Phenylphenyl)methylthio]phenyl}amino) thioxomethyl]amino}vinyl)phenyl]prop-2-enoic acid was prepared using the procedure for Example 2 from hydrazino(1{3-[(2-phenylphenyl)methylthio]phenyl}amino)methane-1-thione (183 mg; 0.5 mmol) and 4-formylcinnamic acid (88 mg; 0.5 mmol) to obtain a yellow solid.

Yield: 197 mg (75%). TLC (silica gel): R_{f}=0.43.

Ethyl (2E)-3-{4-[5-({3-[(2-phenylphenyl)methylthio]phenyl}amino)(1,3,4-thiadiazol-2-yl)]phenyl}prop-2-enoate was prepared using the procedure for Example 2 from (2E)-3-[4-(1E)-2-aza-2-{[({3-[(2-phenylphenyl)methylthio]phenyl}amino)thioxomethyl]amino}vinyl)phenyl]prop-2-enoic acid (130.9 mg; 0.25 mmol) and iron(III) chloride hexahydrate (203 mg; 0.75 mmol) to obtain a yellow solid. Yield: 88 mg (64%). TLC (silica gel): R_{f}=0.56.

The title compound was prepared using the procedure for Example 7 from ethyl (2E)-3-{4-[5-({3-[(2-phenylphenyl)methylthio]phenyl}amino) (1,3,4-thiadiazol-2-yl)]phenyl}prop-2-enoate (70 mg; 0.13 mmol) and lithium hydroxide (24 mg; 1 mmol). Yield: 56 mg (84%). mp 255-257° C. Mass (APCI): (MH)⁺: 522 (Calc.); 522 (Obsd.).

### Example 22

[4-(3,4-dichlorophenyl)(1,3-thiazol-2-yl)](3-bromophenyl)amine

To a solution of amino[(3-bromophenyl)amino]methane-1-thione (10 mg; 4.32 mmol) in dioxane (3 mL) at RT was added 1-(3,4-dichlorophenyl)-2-bromoethan-1-one (120 mg; 4.5 mmol). The reaction mixture stirred at RT for one hour and pyridine (0.4 mL; 4.66 mmole) was added. After refluxing for five hours, the reaction mixture was concentrated under reduced pressure, quenched with water, and the resulting mixture was extracted with ethyl acetate. The organic layer was washed with water, with brine, and dried over anhydrous sodium sulfate. Concentration under reduced pressure afforded a crude product that was purified by flash chromatography (50/50 Ethyl acetate/Hexane) to yield the title compound (135 mg; 78%) as a liquid. The product had a HPLC retention time of 3.90 minutes (Column: C18, 5 μm, 50×3 mm); Solvent A=(H2O, 10% AcOH); solvent B=(Acetonitrile, 10% AcOH) and LC/SM (SSQ-7000) M+=400.

### Example 23

5-{3-[3-(trifluoromethyl)phenoxy]phenyl})-1,3,4-thiadiazole-2-ylamine

The reactions described in Example 2 were repeated, using aminohydrazinomethane-1-thione (1.82 g; 20.0 mmol) and 3-[3-(trifluoromethyl)phenoxy]benzaldehyde (5.32 g; 20.0 mmol) to yield [(1-aza-2-{3-[3-(trifluoromethyl)phenoxy]phenyl}vinyl)amino]aminomethane-1-thione (6.43 g; 95%) in the first step.

In the second step, [(1-aza-2-{3-[3-(trifluoromethyl)phenoxy]phenyl}vinyl)amino]aminomethane-1-thione (6.35 g; 18.7 mmol) and iron (III) chloride hexahydrate (12.16 g; 45.0 mmol) were used to yield the title compound (2.42 g; 38%). mp 214-215° C.; Mass (M)⁺337 (Calc.); 337 (Obsd.).

### Example 24

4-{2-[(3-bromophenyl)amino]-1,3-thiazol-4-yl}phenyl benzoate

To a solution of amino[(3-bromophenyl)amino]methane-1-thione (100 mg; 4.32 mmol) in dioxane (3 mL) at RT was added 4-(2-bromoacetyl)phenyl benzoate (143 mg; 4.5 mmol). The reaction mixture stirred at RT for one hour and pyridine (0.4 mL; 4.66 mmole) was added. After refluxing for five hours the reaction mixture was concentrated under reduced pressure, quenched with water, and the resulting mixture was extracted with ethyl acetate. The organic layer was washed with water, with brine, and dried over anhydrous sodium sulfate. Concentration under reduced pressure afforded a crude product that was purified by flash chromatography (50/50 Ethyl acetate/Hexane) to yield the title compound (146 mg; 75%) as a white solid. mp from ethyl acetate: 168-170° C. The product had a HPLC retention time of 3.57 min (Column: C18, 5 μm, 50×3 mm); Solvent A=(H₂O, 10% AcOH); solvent B (Acetonitrile, 10% AcOH) and LC/SM (SSQ-7000) M+=452.

### Example 25

3-{5-[(3,4-dichlorophenyl)amino](1,3,4-thiadiazol-3-yl)}phenyl 3-(trifluoromethyl)phenyl ketone

To the product from Procedure E (500 mg; 1.5 mmol) and celite (11.0 g) in dichloromethane (4 mL) was add pyridinium chlorochromate (970 mg; 4.5 mmol). The mixture was stirred for two hours, filtered, and the filter cake was rinsed with additional dichloromethane and the filtrate concentrated in vacuo to an oily residue. The residue was subjected to silica gel chromatography (1/9 ethyl acetate/hexane) to yield 3-(1,3-dioxolan-2-yl)phenyl 3-(trifluoromethyl)phenyl ketone (442 mg; 91%).

This intermediate was hydrolyzed to the 3-{[3-(trifiuoromethyl)phenyl]carbonyl}benzaldehyde by stirring in 1N HCl/Dioxane (1/1) for two hours at 25° C. The aldehyde (154 mg, 0.56 mmol) and the product from Procedure D (132 mg; 0.56 mmol) were then combined as in Example 2 and recrystallized from ethanol to yield the title compound.

Yield 175 mg (63%). mp (uncorrected): 216-217° C.

### Example 26

N-{(1Z-2-aza-2-3,4-dichlorophenyl)-1-[(4-(1,2,3-thiadiazol-4-yl)phenyl)methylthio]vinyl}[3-(trifluoromethyl)phenyl]carboxamide

A solution of amino[(3,4-dichlorophenyl)amino]methane-1-thione (145 mg; 0.65 mmol) and 4-[4-(bromomethyl)phenyl]-1,2,3-thiadiazole in dioxane (4 mL) was refluxed for four hours. Most of the solvent was evaporated under vacuum, and the residue was washed with ether (3×20 mL) to provide the HBr salt of {amino[(4-(1,2,3-thiadiazol-4-yl)phenyl)methylthio]methyl}(3,4-dichlorophenyl)amine (248 mg; 80%) as a yellow solid.

¹HNMR (300 MHz, CDCl₃); 11.31(s, 1H); 8.69(s, 1H); 8.08-8.05(d, J=9 Hz, 1H); 7.59-7.20 (m, 7H); 4.68 (s, 2H).

To a solution of this salt (116 mg; 0.23 mmol) in dry pyridine (5 mL) was added 3-(trifluoromethyl)benzoyl chloride at RT. The resulting solution was refluxed for 15 minutes. The cold reaction mixture is poured into the water (10 mL). The precipitate is filtered, washed with water, and recrystallized from ethanol to yield the title compound as a white solid. Mass spec (APCI): M+=566: mp 150-155° C.

### Example 27

1-(4-{[5-(3-nitrophenyl)-1,3,4-thiadiazol-2-yl]amino}phenyl)ethan-1-one

The title compound was synthesized from 3-nitro-benzenecarbohyrazide (181 mg; 1 mmol) and 4-acetylbenzenisothiocyanate (197 mg; 1.1 mmol) by the procedure of Example 1. recrystallized from ethanol/water. Yield: 38% (isolated) Mass spec: (MH⁺) 341.1 (NuMega)

### Example 28

Ethyl 2-(3-{5-[(3-bromophenyl)amino]-1,3,4-thiadiazol-2-yl}phenoxy)acetate

The title compound was synthesized from [(3-bromophenyl)amino]hydrazinometlhane-1-thione (80 mg; 0.47 mmol) and 2-(3-carbonylphenoxy) acetic acid (10 mg; 0.43 mmol) by the procedure of Example 2. Yield: 62% (isolated) mp 175° C. (uncorrected) from ethyl acetate/hexane.

### Example 29

(3,4-dichlorophenyl)[5-(3,5-dinitrophenyl)(1,3,4-thiadiazol-2-yl)]amine

The title compound was synthesized from 3,5-dinitrobenzoyl chloride (50 mg; 0.25 mmol) and the product from Procedure D (59 mg; 0.25 mmol) by the procedure of Example 3. mp 305-306.5° C. (uncorrected) from ethanol. Yield: 61% (isolated)

### Example 30

[5-(4-nitrophenyl)(1,3-thiazol-2-yl)](4-phenoxyphenyl)amine

The title compound was synthesized from amino[(4-phenoxyhenyl)amino]methane-1-thione (228 mg; 0.9 mmol) and 2-bromo-1-(4-nitrophenyl)ethan-1-one (240 mg; 1.2 mmol) by the procedure of Example 4. Yield: 45% (isolated) mp 191-196° C. (uncorrected) from ethyl acetate/hexane

### Example 31

Methyl 3-(aza(5-(3-ethoxyphenyl]-3-[4-phenylphenyl)methyl(1,3,4-thiadiazolin-2-ylidene)}methyl)benzoate

The title compound was synthesized from methyl-3{[5-(3-ethoxyphenyl)-1,3,4-thiadiazol-2-yl]amino}benzoate and 4-(bromomethyl)-1-phenylbenzene by the procedure in Example 8 and purified using silica gel chromatography as the less polar of the two isomers. Mass: (MH+)⁺552.07 SBI APCI+Q1 ms

### Example 32

Ethyl 2-[(1Z & 1E)-1-aza-2-(3-{5-[(3,4-dichlorophenyl)amino](1,3,4-thiadiazol-3-yl)}phenyl)-2-[3-(trifluoromethyp)phenyl]vinyloxy]acetate

To the product from Example 25 (168 mg; 0.34 mmol) was added carboxymethoxylamine hydrochloride (43 mg; 0.34 mmol) in ethanol. The reaction mixture was refluxed for six hours, then allowed to cool to 0° C. The precipitate was collected and recrystallized from ethanol to yield the product (75 mg; a mixture of cis and trans). Yield 37% (isolated). mp 170-179° C.

### Example 33

1-(4-{[5-(3-nitrophenyl)-1,3,4-thiadiazol-2-yl]amino}phenyl)ethan-1-one

The title compound was synthesized from 4-acetylphenyl isothiocyanate and 3-nitrobenzoylhydrazide using the procedure in Example 1. Yield: 38% (isolated) Mass: (MH+)⁺341.1

### Example 34

[5-(4-nitrophenyl)(1,3-thiazol-2-yl)](4-phenoxyphenyl)amine

4-phenoxybenzenisothiocyanate (1.0 g; 4.4 mmol) and 0.5M ammonia (30 mL; 15 mmol) in dioxane were combined as in Example 4 to yield (amino[(4-phenoxyphenyl)amino]methane-1-thione) in 83% yield. Without purification, this intermediate was combined with 4-nitrophenyl-2′-bromoacetophenone as in Example 4 to yield the title compound in 45% yield. mp 191-196° C. (uncorrected); Yield: 45% (isolated).

### Example 35

3-{2-[Aza(3,4-dichlorophenyl)methylene]-3-methyl(1,3,4-thiadiazolin-5-yl))-1-[3-(trifluoromethyl)phenoxy]benzene

and

(3,4-Dichlorophenyl)methyl(5-{3-[3-(trifluoromethyl)phenoxy]phenyl}(1,3,4-thiadiazol-2-yl))amine

The reactions described in Example 8 were repeated, but using (3,4-dichlorophenyl)(5-{3-[3-(trifluoromethyl)phenoxy]phenyl}(1,3,4-thiadiazol-2-yl))amine (241 mg; 0.5 mmol), potassium t-butoxide (0.55 mL; 0.55 mmol; 1M in THF) and of iodomethane (0.12 mL; 2 mmol) to yield 3-{2-[Aza(3,4-dichlorophenyl)methylene]-3-methyl(1,3,4-thiadiazolin-5-yl)}-1-13-(trifiuoromethyl)phenoxy]benzene (41 mg; 17%, the less polar of the two isomers) with the following physical properties: mp 78-79° C.; MS m/z 496 (M⁺) (LC/MS SSQ.7000).

The more polar isomer, (3,4-Dichlorophenyl)methyl(5-{3-[3-(trifluoromethyl)phenoxy]phenyl}(1,3,4-thiadiazol-2-yl))amine, was isolated in a yield of 20 mg (8%), with the following physical properties: MS m/z 496 (M⁺) (LC/MS SSQ.7000);

¹HNMR (300 MHz, CDCl₃) 3.63 (s, 3H), 7.07 (d, J=8.7 Hz, 1H), 7.19 (d, J=8.1 Hz, 1H), 7.26-7.53 (m, 9H).

### Example 36

(3-Bromophenyl){5-[(2-nitrophenoxy)methyl](1,3,4-thiadiazol-2-yl)}amine 2-(2-nitrophenoxy)acetohydrazide (211 mg; 1 mmol) and 3-bromophenyl isothiocyanate (235 mg; 1.1 mmol) were combined as in Example 1 to form the title compound (193 mg; 81% yield): mp 184-185° C.;

¹H NMR (300 MHz, DMSO-d₆) δ 5.64 (s, 2H), 7.19 (d, J=6.0 Hz, 2H), 7.31 (t, J=6.9 Hz, 1H), 7.47 (d, J=8.4 Hz, 1H), 7.55 (d, J=9.0 Hz, 1H), 7.70 (t, J=6.6 Hz, 1H), 7.92 (d, J=6.9 Hz, 1H), 8.05 (s, 1H), 10.67 (s, 1H); MS m/z 407 (MN; R_{f} 0.65 (EtOAc, silica).

### Example 37

3-({5-[3,5-bis(phenylmethoxy)phenyl]-1,3,4-thiadiazol-2-yl}amino)benzoic acid

3-[(hydrazinothioxomethyl)amino]benzoic acid was prepared using Procedure F from 3-carboxyphenyl isothiocyanate (896 mg; 5 mmol) and hydrazine hydrate (485 μL; 10 mmol) and was used for next reaction without further purification.

¹H NMR: (300 MHz, DMSO-d₆) 9.11 δ (1H, b); 8.07 δ (1H, s); 7.73 δ (1H, d); 6.23 δ (1H, d); 7.23 δ (1H, t); 7.00 δ (2H, b); 4.84 δ (1H, b).

The title compound was prepared using the procedure for Example 2 from 3-[(hydrazinothioxomethyl)amino]benzoic acid (845 mg; 4 mmol) and 3,5-dibenzyloxy benzaldehyde (1.27 g; 4 mmol). Yield: 152 mg (10%). Mass: (MH)⁺: 510

### Example 38

1-{6-[3-(2,4-Dichlorophenyl)isoxazol-5-yl]imidazolo[2,1-b]1,3,4-thiadiazolin-2-yl}-3-methoxybenzene

The title compound was prepared as in Example 16 from 5-(3-methoxy phenyl)-1,3,4-thiadiazole-2-ylamine (103 mg; 0.5 mmol) and 5-(bromoacetyl)-3-(2,4-dichlorophenyl)isoxazole (168 mg; 0.5 mmol). Yield: 75 mg (34%). mp 195-198° C. Mass: (M)⁺: 443 (Calc.); 443 (Obsd.).

### Example 39

2-(3-Nitrophenyl)-6-(4-phenylphenyl)imidazolo[2,1-b]1,3,4-thiadiazoline 5-(3-Nitrophenyl)-1,3,4-thiadiazole-2-ylamine was prepared using the procedure for Example 16 from 3-nitro benzoic acid (5.01 g; 30 mmol) and thiosemicarbozide (2.73 g; 30 mmol) and phosphorous oxychloride (9 mL). Yield: 3.29 g (50%). mp 205-206° C. Mass: (M+2H)⁺: 224 (Calc.), 224 (Obsd.).

2-(3-Nitrophenyl)-6-(4-phenylphenyl)imidazolo[2,1-b]1,3,4-thiadiazoline: The title compound was prepared using the procedure of Example 16 using 5-(3-nitrophenyl)-1,3,4-thiadiazole-2-ylamine (111 mg; 0.5 mmol) and 2-bromo-4′-phenyl acetophenone (138 mg; 0.5 mmol). Yield: 75 mg (19%). mp 263-265° C. Mass: (MH)⁺: 399 (Calc.); 399 (Obsd.).

### Example 40

6-(2H,3H,4EI-Benzo[b]1,4-dioxepan-7-yl)-2-(3-nitrophenyl)imidazolo [2,1-b]1,3,4-thiadiazoline

The title compound was prepared using the procedure of Example 16 using 5-(3-nitrophenyl)-1,3,4-thiadiazole-2-ylamine (111 mg; 0.5 mmol) and 3,4-(Trimethylenedioxy)phenacyl bromide (136 mg; 0.5 mmol). Yield: 75 mg (18%). mp 227-230° C. Mass: (M⁺: 395 (Calc.); 395 (Obsd.).

### Example 41

{3-[(4-Methoxyphenyl)methylthio]phenyl}[5-(3-nitrophenyl)(1,3,4-thiadiazol-2-yl)]amine

3-(4-Methoxy phenylmethylthio)phenylamine: The title compound was prepared from 4-(chloromethyl)-1-methoxybenzene (4.7 g; 30 mmol) and 3-amino thiophenol (3.75 g; 30 mmol) using Procedure A. The solid was purified using a silica gel column and the title compound was eluted with 20% EtOAc/hexanes. Yield: 4.5 g (61%).

3-[(4-Methoxyphenyl)methylthio]benzenisothiocyanate was prepared using Procedure C from 3-(4-methoxy phenylmethylthio)phenylamine (1.84 g; 7.5 mmol) and thiophosgene (1.14 mL; 15 mmol) to obtain slightly purple solid. Yield: 2.0 g (93%).

Hydrazino({3-[(4-methoxyphenyl)methylthio]phenyl}amino)methane-1-thione was prepared from 3-(phenylmethylthio) benzenisothiocyanate (290 mg; 1 mmol) and hydrazine hydrate (0.1 mL; 2 mmol) using Procedure D. Yield: 275 mg (86%).

[(1E)-1-Aza-2-(3-nitrophenyl)vinyl]amino}({3-[(4-methoxyphenyl)methylthio]phenyl}amino)methane-1-thione was prepared from hydrazino({3-[(4-methoxyphenyl) methylthio]phenyl}amino)methane-1-thione (240 mg; 0.75 mmol) and 3-nitrobenzaldehyde (113 mg; 0.75 mmol) as in Example 2. Yield: 275 mg (81%). TLC (silica gel): R_{f}=0.9 (EtOAc/Hexane-1/1).

The title compound was prepared as in Example 2 from {[(1E)-1-aza-2-(3-nitrophenyl)vinyl]amino}({3-[(4-methoxyphenyl)methylthio]phenyl}amino)methane-1-thione (226 mg; 0.5 mmol) and iron (III) chloride hexahydrate (405 mg; 1.5 mmol). Yield: 176 mg, (78%). mp 208-209° C. Mass spectrum (electrospray): NH)⁺: 451 (Calc.); 451 (Obsd.).

### Example 42

[5-(3-Ethoxyphenyl)(1,3,4-thiadiazol-2-yl)]{3-[(4-phenylphenyl)methylthio]phenyl}amine

3-[(4-Phenylphenyl)methylthio]phenylamine: The title compound was prepared using Procedure A from 3-aminothiophenol (0.4 mL; 3.5 mmol) and 1-(chloromethyl)₄-phenylbenzene (750 mg; 3.5 mmol) as a white solid. Yield: 970 mg (95%). TLC (silica gel): R_{f}=0.4 (EtOAc/Hexane=1/4).

3-[(4-Phenylphenyl)methylthio]benzenisothiocyanate was prepared using Procedure C from 3-[(4-phenylphenyl)methylthio]phenylamine (950 mg; 3.25 mmol) and thiophosgene (0.49 mL; 6.5 mmol). Yield: 910 mg (84%). TLC (silica gel): Rf=0.9 (EtOAc/Hexane=1/4).

Hydrazino({3-[(4-phenylphenyl)methylthio]phenyl}amino)methane-1-thione was prepared using Procedure D from 3-[(4-phenylphenyl)methylthio]benzenisothiocyanate (900 mg; 2.7 mmol) and hydrazine hydrate (0.27 mg; 5.4 mmol) as a white solid. Yield: 750 mg (76%).

{[(1E)-1-Aza-2-(3-ethoxyphenyl)vinyl]amino}({3-[(4-phenylphenyl)methylthio]phenyl}amino)methane-1-thione: The title compound was prepared as in Example 2 from hydrazino({3-[(4-phenylphenyl)methylthio]phenyl}amino)methane-1-thione (274 mg; 0.75 mmol) and 3-ethoxybenzaldehyde (113 mg; 0.75 mmol) to obtain a white solid. Yield: 320 mg (85%).

The title compound was prepared using the procedure for Example 2 from {[(1E)-1-aza-2-(3-methoxyphenyl)vinyl]amino}({3-[(4-phenylphenyl)methylthio]phenyl}amino) methane-1-thione (242 mg; 0.5 mmol) and iron(III) chloride hexahydrate (405 mg, 1.5 mmol). Yield: 172 mg (69%). mp 215-217° C. Mass: (MN)⁺: 496 (Calc.); 496 (Obsd.). AA

### Example 43

[5-3-Nitrophenyl)(1,3,4-thiadiazol-2-yl)]{3-[(4-phenylphenyl)methylthio]phenyl}amine

{[(1E)-1-Aza-2-(3-nitrophenyl)vinyl]amino}({3-[(4-phenylphenyl)methylthio]phenyl}amino)methane-1-thione was prepared using the procedure in Example 2 from hydrazino({3-[(4-phenylphenyl)methylthio]phenyl}amino)methane-1-thione (183 mg; 0.5 mmol) and 3-nitrobenzaldehyde (76 mg; 0.5 mmol) as a white solid. Yield: 210 mg (84%).

The title compound was prepared as in Example 2 from {[(1E)-1-aza-2-(3-nitrophenyl) vinyl]amino}({3-[(4-phenylphenyl)methylthio]phenyl}amino) methane-1-thione (200 mg; 0.4 mmol) and iron(III) chloride hexahydrate (324 mg; 1.2 mmol). Yield: 90 mg (45%). mp 229-231° C. Mass: (MH)⁺: 497 (Calc.); 497 (Obsd.).

### Example 44

[5-(3-Methylphenyl)(1,3,4-thiadiazol-2-yl)](2,4,5-trichlorophenyl)amine

(3-Methylphenyl)-N-({thioxo[(2,4,5-trichlorophenyl)amino]methyl}amino)carboxamide: The title compound was prepared using the procedure for Example 1 from 3-toluic hydrazide (100 mg; 0.67 mmol) and 2,4,5-trichlorophenyl isothiocyanate (160 mg; 0.67 mmol). Yield: 39.2 mg (15%). TLC (silica gel): R_{f}=0.17.

The title compound was prepared using the procedure for Example 1 from (3-methylphenyl)-N-({thioxo[(2,4,5-trichlorophenyl)amino]methyl}amino)carboxamide (79 mg; 0.2 mmol) and sulfuric acid (6 drops). Yield: 16 mg (21.5%). mp 229-231° C. Mass (electrospray): (MH)⁺: 497 (Calc.); 497 (Obsd.).

### Example 45

{5[3,5-Bis(phenylmethoxy)phenyl](1,3,4-thiadiazol-2-yl)}[3-(3-phenylpropylthio) phenyl]amine

3-(3-Phenylpropylthio)phenylamine was prepared from 3-amino thiophenol (10 mmol, 1.06 mL) and (3-bromopropyl)benzene (1.52 mL; 10 mmol) using Procedure A. The yellow liquid was purified using a silica gel column and the title compound was eluted with 40% hexanes/ethylacetate. Yield: 1.7 mL (74%).

3-(3-Phenylpropylthio)benzenisothiocyanate was prepared using Procedure C from 3-(3-phenylpropylthio)phenylamine (1.8 g; 7.4 mmol) and thiophosgene (1.12 mL; 14.8 mmol) to obtain a brown liquid. Yield: 2.3 g (100%).

Hydrazino {[3-(3-phenylpropylthio)phenyl]amino}methane-1-thione: The title compound was prepared using Procedure F from 3-(3-phenylpropylthio)benzenisothiocyanate (2.3 g; 8 mmol) and hydrazine hydrate (0.4 mL; 16 mmol) to obtain a white solid. Yield: 2.11 g (83%). TLC (silica gel): R_{f}0.25.

({(1E)-1-Aza-2-[3,5-bis(phenylmethoxy)phenyl]vinyl}amino){[3-(3-phenylpropylthio) phenyl]amino}methane-1-thione was prepared using the procedure for Example 2 from hydrazino{[3-(3-phenylpropylthio)phenyl]amino}methane-1-thione (159 mg; 0.5 mmol) and 3,5-dibenzyloxybenzaldehyde (159 mg; 0.5 mmol). Yield: 290 mg (94%). TLC (silica gel): R_{f}=0.84.

5-[3,5-bis(phenylmethoxy)phenyl](1,3,4-thiadiazol-2-yl)}[3-(3-phenylpropylthio) phenyl]amine: The title compound was prepared as in Example 2 from ({(1E)-1-aza-2-[3,5-bis(phenylmaethoxy)phenyl]viny}amino) {[3-(3-phenylpropylthio)phenyl]amino}methane-1-thione (154 mg; 0.25 mmol) and iron(III) chloride hexahydrate (202 mg; 0.75 mmol). Yield: 124 mg (81%). mp 160-162° C. Mass: (W)⁺: 616 (Calc.); 616 (Obsd.). AA

### Example 46

{5-[3,5-Bisphenylmethoxy)phenyl](1,3,4-thiadiazol-2-yl)}{3-[(2-phenylphenyl) methylthio]phenyl}amine

3-[(2-Phenylphenyl)methylthio]phenylamine was prepared from 3-amino thiophenol (10 mmol, 1.06 mL) and 2-phenylbenzyl bromide (1.83 mL; 10 mmol) using Procedure A. The yellow liquid was purified using a silica gel column and the title compound was eluted with 40% hexanes/ethylacetate. Yield: 1.8 mL (62%). TLC (silica gel): R_{f}=0.72

3-[(2-Phenylphenyl)methylthio]benzenisothiocyanate was prepared using Procedure C from 3-[(2-phenylphenyl)methylthio]phenylamine (1.8 g; 6 mmol) and thiophosgene (0.91 mL; 12 mmol) to obtain a brown liquid. Yield: 2.2 g (90%). TLC (silica gel): R_{f}0.69.

Hydrazino({3-[(2-Phenylphenyl)methylthio]phenyl}amino)methane-1-thione was prepared using Procedure F from 3-[(2-phenylphenyl)methylthio]benzenisothiocyanate (2.2 g, 6.6 mmol) and hydrazine hydrate (0.33 mL; 13.2 mmol) to obtain a white solid. Yield: 1.91 g (79%). TLC (silica gel): R_{f}=0.28.

({(1E)-1-Aza-2-[3,5-bis(phenylmethoxy)phenyl]vinyl}amino)({3-[(2-phenylphenyl) methylthio]phenyl}amino)methane-1-thione: The title compound was prepared using the procedure for Example 2 from hydrazino({3-[(2-phenylphenyl)methylthio]phenyl}amino)methane-1-thione (183 mg; 0.5 mmol) and 3,5-dibenzyloxybenzaldehyde (159 mg; 0.5 mmol) to obtain a white solid. Yield: 132 mg (40%). TLC (silica gel): R_{f}=0.84

The title compound was prepared using the procedure for Example 2 from ({(1E)-1-aza-2-[3,5-bis(phenylmethoxy)phenyl]vinyl}amino)({3-[(2-phenylphenyl) methylthio]phenyl}amino)methane-1-thione (120 mg; 0.18 mmol) and iron(III) chloride hexahydrate (146 mg; 0.54 mmol). Yield: 85 mg (71%). mp 128-130° C. Mass (electrospray): (MH)⁺: 664 (Calc.); 664 (Obsd.)⁺.

### Example 47

{3-[(2-Phenylphenyl)methylthio]phenyl}{5-[2-(trifluoromethyl)phenyl](1,3,4-thiadiazol-2-yl)}amine

({(1E)-1-Aza-2-[2-(trifluoromethyl)phenyl]vinyl}amino)({3-[(2-phenylphenyl). methylthio]phenyl}amino)methane-1-thione was prepared using the procedure for Example 2 from hydrazino({3-[(2-phenylphenyl)methylthio]phenyl}amino)methane-1-thione (183 mg; 0.5 mmol) and 2-(trifluoromethyl) benzaldehyde (0.7 mL; 0.5 mmol) to obtain a white solid.

Yield: 104 mg (40%). TLC (silica gel): R_{f}=0.84.

{3-[(2-Phenylphenyl)methylthio]phenyl}{5-[2-(trifluoromethyl)phenyl](1,3,4-thiadiazol-2-yl)}amine was prepared using the procedure for Example 2 from ({(1E)-1-aza-2-[2-(trifluoromethyl)phenyl]vinyl}amino)({3-[(2-phenylphenyl)methylthio]phenyl}amino) methane-1-thione (94 mg; 0.18 mmol) and iron(III) chloride hexahydrate (146 mg; 0.54 mmol).

Yield: 50 mg (53%). mp 73-75° C. Mass (electrospray): (MH)⁺: 520.

### Example 48

[5-(3-Nitrophenyl)(1,3,4-thiadiazol-2-yl)][3-(3-phenylpropylthio)phenyl]amine

The intermediate {[(1E)-1-Aza-2-(3-nitrophenyl)vinyl]amino}{[3-(3-phenylpropylthio) phenyl]amino}methane-1-thione was prepared using the procedure for Example 2 using hydrazino{[3-(3-phenylpropylthio)phenyl]amino}methane-1-thione (159 mg; 0.5 mmol) and 3-nitrobenzaldehyde (76 mg; 0.5 mmol) as a yellow solid. Yield: 194 mg (86%). TLC (silica gel): R_{f}=0.78.

The title compound was prepared using the procedure for Example 2 from {[(1E)-1-aza-2-(3-nitrophenyl)vinyl]amino}{[3{3-phenylpropylthio)phenyl]amino}methane-1-thione (112.6 mg; 0.25 mmol) and iron(III) chloride hexahydrate (203 mg; 0.75 mmol). Yield: 945 mg (85%). mp 169-171° C. Mass: (MH)⁺: 449 (Calc.); 449 (Obsd.). AA

### Example 49

4-({5-[3,5-bis(phenylmethoxy)phenyl]-1,3,4-thiadiazol-2-yl}amino)benzole acid

Methyl 4-[hydrazinothioxomethyl)amino]benzoate was prepared using Procedure F from 4-methoxycarbonylphenyl isothiocyanate (193 mg; 1 mmol) and hydrazine hydrate (0.1 mL; 2 mmol) as a white solid. Yield: 192 mg (85%).

Methyl 4-{[({(1E)-1-aza-2-[3,5-bisphepnylmethoxy)phenyl]vinyl}amino)thioxomethyl]amino}benzoate was prepared using the procedure for Example 2 from methyl 4-[(hydrazinothioxomethyl) amino]benzoate (180 mg; 0.8 mmol) and 3,5-dibenzyloxybenzaldehyde (255 mg; 0.8 mmol) as a white solid. Yield: 302 mg (72%).

Methyl 4-({5-[3,5-bis(phenylmethoxy)phenyl]-1,3,4-thiadiazol-2-yl}amino)benzoate was prepared using the procedure for Example 2 from methyl 4-{[({(1E)-1-aza-2-[3,5-bis(phenyhnethoxy)phenyl]vinyl}amino) thioxomethyl]amino}benzoate (289 mg; 0.55 mmol) and iron(III) chloride hexahydrate (446 mg; 1.65 mmol). Yield: 226 mg (78%). TLC (silica gel): R_{f}=0.69.

The title compound was prepared as in Example 7 using methyl 4-({5-[3,5-bis(phenylmethoxy)phenyl]-1,3,4-thiadiazol-2-yl}amino)benzoate (100 mg; 0.19 mmol) and lithium hydroxide (30 mg; 1.25 mmol). Yield: 94 mg (97%). mp 297-299° C. Mass (APCI): (MH)⁺: 510 (Calc.); 510 (Obsd.).

### Example 50

Ammonium (2E)-3-{4-[5-({3-[(2-phenylphenyl)methylthio]phenyl}amino) (1,3,4-thiadiazol-2-yl)]phenyl}prop-2-enoate

The product from Example 21 (20.5 mg; 0.04 mmol) was dissolved in ammonium hydroxide (5 mL) and water (5 mL). The mixture was stirred until all solid was in solution. The solvents were removed in vacuo to obtain the title compound as a yellow solid. Yield: 18.5 mg (87%). Mass: (M⁺: 522 (Calc.); 522 (Obsd).

### Example 51

{5-[3,5-bis(phenylmethoxy)phenyl](1,3,4-thiadiazol-2-yl)}){4[3-(trifluoromethyl)phenoxy]phenyl}amine

4-[3-(trifluoromethyl)phenoxy]benzenisothiocyanate was prepared using Procedure C from 4-[3-(trifluoromethyl)phenoxy]phenylamine (883 mg; 3.5 mmol) and thiophosgene (0.53 mL; 7 mmol) as a brown liquid. Yield: 900 mg (87%).

Hydrazino({4-[3-(trifluoromethyl)phenoxy]phenyl}amino)methane-1-thione was prepared using Procedure F from 4-[3-(trifluoromethyl) phenoxy]benzenisothiocyanate (800 mg; 2.7 mmol) and hydrazine hydrate (0.26 mL; 5.4 mmol) as a white solid. Yield: 737 mg (83%).

({(1E)-1-Aza-2-[3,5-bis(phenylmethoxy)phenyl]vinyl}amino)({4-[3-trifluoromethyl) phenoxy]phenyl}amino)methane-1-thione was prepared by the procedure for Example 2 using hydrazino({4-[3-(trifluoromethyl)phenoxy]phenyl}amino) methane-1-thione (327 mg; 1 mmol) and 3,5-dibenzyloxybenzaldehyde (318 mg; 1 mmol) as a white solid. Yield: 534 mg (85%).

The title compound was prepared by the procedure for Example 2 from. ({(1E)-1-aza-2-[3,5-bisfphenylmethoxy)phenyl]vinyl}amino)(14-[3-(trifluoromethyl)phenoxy]phenylamino) methane-1-thione (377 mg; 0.6 mmol) and iron(I) chloride hexahydrate (486 mg; 1.8 mmol).

Yield: 201 mg (53%). mp 181-183° C. Mass (APCI): (M⁺: 626 (Calc.); 626 (Obsd.).

### Example 52

2-{4-[3-({5-[3,5-bis(phenylmethoxy)phenyl]-1,3,4-thiadiazol-2-yl}amino)phenoxy]phenyl}acetic acid

Methyl 2-[4-(3-nitrophenoxy)phenyl]acetate was prepared using Procedure B from methyl-4-hydroxy phenylacetate (3.32 g; 20 mmol) and 3-bromo nitrobenzene (4.04 g; 20 mmol), Copper oxide (3.19 g; 40 mmol) and potassium carbonate (5.54 g; 40 mmol) in pyridine (20 mL).

Yield: 1.4 g (25%).

Methyl 2-[4-(3-isothiocyanatophenoxy)phenyl]acetate was prepared as in Procedure C from the product of Procedure H (1.16 g; 4.5 mmol) and thiophosgene (0.69 mL; 9 mmol) in methylene chloride. Yield: 1.3 g (97%). TLC (silica gel): R_{f}=0.9 (EtOAc/Hexane=1/1).

Methyl 2-(4-{3-[(hydrazinothioxomethyl)amino]phenoxy}phenyl)acetate was prepared using Procedure F from methyl 2-[4-(3-isothiocyanato-phenoxy)phenyl]acetate (1.1 g; 3.6 mmol) and hydrazine hydrate (0.35 mL; 7.2 mmol) in toluene (10 mL). Yield: 1.05 g (88%).

¹H NMR: (300 MHz, DMSO-d₆) 9.20δ (1H, s); 7.68δ (1H, s); 7.29δ (5H, m); 6.98δ (2H, d); 6.71δ (1H, d); 3.67δ (2H, s) 3.62δ (3H, s).

Methyl 2-[4-(3-{[({(1E)-1-aza-2-[3,5-bis(phenylmethoxy)phenyl]vinyl}amino) thioxomethyl]amino}phenoxy)phenyl]acetate was prepared as in Example 2 from methyl 2-(4-{3-[(hydrazinothioxomethyl)amino]phenoxy}phenyl)acetate (331 mg; 1 mmol) and 3,5 dibenzyloxy benzaldehyde (318 mg; 1 mmol) in ethanol (5 mL). Yield: 490 mg (77%). Mass (APCI): (M⁺: 632.

Methyl 2-{4-[3-({5-[3,5-bis(phenylmethoxy)phenyl]-1,3,4-thiadiazol-2-yl}amino) phenoxy]phenyl}acetate was prepared using the procedure as in Example 2 from methyl 2-[4-(3-{[({(1E)-1-aza-2-[3,5-bis(phenylmethoxy)phenyl]vinyl}amino)thioxomethyl]amino}phenoxy)phenyl]acetate (379 mg; 0.6 mmol) and Iron chloride (486 mg; 1.8 mmol) in Ethanol. Yield: 146 mg (37%). Mass (APCI): (MH)⁺: 630.

The title compound was prepared as in Example 7 from methyl 2-{4-[3-({5-[3,5-bis(phenylmethoxy)phenyl]-1,3,4-thiadiazol-2-yl}amino) phenoxy]phenyl}acetate (100 mg; 0.16 mmol) and lithium hydroxide (24 mg; 1 mmol). Yield: 33 mg (34%).

¹H NMR: (300 MHz, DMSO-d₆) 12.30δ (1H, b); 10.66δ (1H, s); 7.38δ (15H, m); 7.28δ (4H, d); 6.82δ (1H, s); 6.25δ (1H, d) 5.18δ (4H, s); 3.57δ (2H, d). Mass: (MH)⁺: 616 (Calc.); 616 (Obsd.).

### Example 53

N2-(3-chloro-4-bromophenyl)-5-(3-ethoxyphenyl)-1,3,4-thiadiazol-2-amine

3-chloro-4-bromophenyl isothiocyanate and 3-ethoxybenzhydrazide were combined as described in Example 1 to yield the title compound with an overall yield of 69%.

¹H NMR (DMSO-d₆) δ 1.36 (t, 3H), 4.12 (q, 2H), 7.08 (m, 1H), 7.43 (m, 4H), 7.72 (d, 1H), 8.13 (d, 1H).

¹³C NMR (DMSO-d₆) δ 14.6, 63.3, 112.1, 112.7, 116.6, 117.8, 118.6, 119.4, 130.5, 131.2, 133.3, 134.0, 140.8, 158.5, 158.9, 163.4.

### Example 54

(3-nitrophenyl)[5-(3-nitrophenyp)(1,3,4-thiadiazol-2-yl)]amine

The title compound was prepared as described in Example 1 from 3-nitrobenzenecarbohydrazide (500 mg) and 3-nitrobenzenisothiocyanate (500 mg). The title compound had the following physical properties: mp 325-330° C. (decomposition).

### Example 55

(2-chloro-5-nitrophenyl)[5-(3-ethoxyphenyl)(1,3,4-thiadiazol-2-yl)]amine

The title compound was prepared as described in Example 1 from 2-chloro-5-nitrobenzenisothiocyanate (100 mg) and 3-ethoxybenzenecarbohydrazide (90 mg). The title compound had the following physical properties: mp 128-130° C.

### Example 56

[5-(3-methoxyphenyl)(1,3,4-thiadiazol-2-yl)](3-nitrophenyl)amine

The title compound was prepared as described in Example 1 from 3-nitrobenzenisothiocyanate (100 mg) and 3-methoxybenzenecarbohydrazide (100 mg). The title compound had the following physical properties: mp 206-208° C.

### Example 57

[5-(3-ethoxyphenyl)(1,3,4-thiadiazol-2-yl)](3-nitrophenyl)amine

The title compound was prepared as described in Example 1 from 3-nitrobenzenisothiocyanate (100 mg) and 3-ethoxybenzenecarbohydrazide (100 mg). The title compound had the following physical properties: mp 155-157° C.

### Example 58

[5-(3-methylphenyl)(1,3,4-thiadiazol-2-yl)](3-nitrophenyl)amine

The title compound was prepared as described in Example 1 from 3-nitrobenzenisothiocyanate (100 mg) and 3-methylbenzenecarbohydrazide (90 mg). The title compound had the following physical properties: mp 219-221° C.

### Example 59

[5-3-nitrophenyl)(1,3,4-thiadiazol-2-yl)][3-(trifluoromethyl)-phenyl]amine

The title compound was prepared as described in Example 1 from 3-(trifluoromethyl)benzenisothiocyanate (250 mg) and 3-nitrobenzenecarbohydrazide (200 mg). The title compound had the following physical properties: mp 266-268° C.

### Example 60

(4-ethylphenyl)[5-(3-nitrophenyl)(1,3,4-thiadiazol-2-yl)]amine

The title compound was prepared as described in Example 1 from 3-ethylbenzenisothiocyanate (200 mg) and 3-nitrobenzenecarbohydrazide (200 mg). The title compound had the following physical properties: mp 208-210° C.

### Example 61

(3-methoxyphenyl) [5-(3-nitrophenyl)(1,3,4-thiadiazol-2-yl)]amine

The title compound was prepared as described in Example 1 from 3-methoxybenzenisothiocyanate (220 mg) and 3-nitrobenzenecarbohydrazide (200 mg). The title compound had the following physical properties: mp 207-209° C.

### Example 62

(4-nitrophenyl)[5-3-nitrophenyl)(1,3,4-thiadiazol-2-yl)]amine

The title compound was prepared as described in Example 1 from 4-nitrobenzenisothiocyanate (220 mg) and 3-mitrobenzenecarbohydrazide (200 mg). The title compound had the following physical properties: mp 327-329° C.

### Example 63

(3-bromophenyl) [5-(3-methoxyphenyl)(1,3,4-thiadiazol-2-yl)]amine

The title compound was prepared as described in Example 1 from 3-bromobenzenisothiocyanate (320 mg) and 3-methoxybenzenecarbohydrazide (250 mg). The title compound had the following physical properties: LC-MS 364.6.

### Example 64

(2,5-dibromophenyl)[5-(3-nitrophenyl)(1,3,4-thiadiazol-2-yl)]amine

The title compound was prepared as described in Example 1 from 2,5-dibromobenzenisothiocyanate (300 mg) and 3-nitrobenzenecarbohydrazide (190 mg). The title compound had the following physical properties: LC-MS 457.4.

### Example 65

(4-bromophenyl)[5-(3-nitrophenyl)(1,3,4-thiadiazol-2-yl)]amine

The title compound was prepared as described in Example 1 from 4-bromobenzenisothiocyanate (500 mg) and 3-nitrobenzenecarbohydrazide (400 mg). The title compound had the following physical properties: LC-MS 377.4.

### Example 66

(4-bromo-3-chlorophenyl)[5-(3-ethoxyphenyl)(1,3,4-thiadiazol-2-yl)]amine

The title compound was prepared as described in Example 1 from 4-bromo-3-chlorobenzenisothiocyanate (500 mg) and 3-ethoxybenzenecarbohydrazide (300 mg). The title compound had the following physical properties: mp 204-205° C.; LC-MS 410.6.

### Example 67

(3-chloro-4-fluorophenyl)[5-(3-nitrophenyl)(1,3,4-thiadiazol-2-yl)]amine

The title compound was prepared as described in Example 1 from 3-chloro-4-fluorobenzenisothiocyanate (570 mg) and 3-nitrobenzenecarbohydrazide (500 mg). The title compound had the following physical properties: LC-MS 351.3.

### Example 68

(3-chloro-4-fluorophenyl)[5-(3-methoxyphenyl)(1,3,4-thiadiazol-2-yl)]amine

The title compound was prepared as described in Example 1 from 3-chloro-4-fluorobenzenisothiocyanate (620 mg) and 3-methoxybenzenecarbohydrazide (500 mg). The title compound had the following physical properties: mp 185-187° C.; LC-MS 336.7.

### Example 69

(4-bromo-3-chlorophenyl)[5-(3-methoxyphenyl)(1,3,4-thiadiazol-2-yl)]amine

The title compound was prepared as described in Example 1 from 4-bromo-3-chlorobenzenisothiocyanate (650 mg) and 3-methoxybenzenecarbohydrazide (400 mg). The title compound had the following physical properties: mp 215-217° C.; LC-MS 398.1.

### Example 70

(3-chloro-4-fluorophenyl)[5-(3-ethoxyphenyl)(1,3,4-thiadiazol-2-yl)]amine

The title compound was prepared as described in Example 1 from 3-chloro-4-fluorobenzenisothiocyanate (260 mg) and 3-ethoxybenzenecarbohydrazide (250 mg). The title compound had the following physical properties: mp 170-172° C.; LC-MS 350.5.

### Example 71

(4-bromo-3-methylphenyl)[5(3-ethoxyphenyl)(1,3,4-thiadiazol-2-yl)]amine

The title compound was prepared as described in Example 1 from 4-bromo-3-methylbenzeriisothiocyanate (700 mg) and 3-ethoxybenzenecarbohydrazide (500 mg). The title compound had the following physical properties: mp 201-202° C.; LC-MS 392.1.

### Example 72

(4-bromo-3-methylphenyl)[5-3-methoxyphenyl)(1,3,4-thiadiazol-2-yl)]amine

The title compound was prepared as described in Example 1 from 4-bromo-3-methylbenzenisothiocyanate (760 mg) and 3-methoxybenzenecarbohydrazide (500 mg). The title compound had the following physical properties: mp 190-192° C.; LC-MS 378.4.

### Example 73

(4-bromo-3-methylphenyl)[5-(3-nitrophenyl)(1,3,4-thiadiazol-2-yl)]amine

The title compound was prepared as described in Example 1 from 4-bromo-3-methylbenzenisothiocyanate (690 mg) and 3-nitrobenzenecarbohydrazide (500 mg). The title compound had the following physical properties: LC-MS 391.4.

### Example 74

(3-bromophenyl)[5-(3-ethoxyphenyl)(1,3,4-thiadiazol-2-yl)]amine

The title compound was prepared as described in Example 1 from 3-bromobenzenisothiocyanate (650 mg) and 3-ethoxybenzenecarbohydrazide (500 mg). The title compound had the following physical properties: mp 195-197° C.; LC-MS 376.5.

### Example 75

Methyl 3-{[5-(3-nitrophenyl)-1,3,4-thiadiazol-2-yl]amino}benzoate

The title compound was prepared as described in Example 1 from methyl 3-isothiocyanatobenzoate (500 mg) and 3-nitrobenzenecarbohydrazide (500 mg). The title compound had the following physical properties: LC-MS 357.5.

### Example 76

(2,3-dichlorophenyl)[5-(3-nitrophenyl)(1,3,4-thiadiazol-2-yl)]amine

The title compound was prepared as described in Example 1 from 2,3-dichlorobenzenisothiocyanate (620 mg) and 3-nitrobenzenecarbohydrazide (500 mg). The title compound had the following physical properties: mp 240-242° C.; LC-MS 367.3.

### Example 77

(3,4-dibromopheny)[5-(3,5-dimethoxyphenyl)(1,3,4-thiadiazol-2-yl)]amine

The title compound was prepared as described in Example 1 from 3,4-dibromobenzenisothiocyanate (280 mg) and 3,5-dimethoxybenzenecarbohydrazide (220 mg). The title compound had the following physical properties: mp 240-242° C.; LC-MS 426.1.

### Example 78

[5-(3-ethoxyphenyl)(1,3,4-thiadiazol-2-yl)](3-nitrophenyl)amine.

The title compound was prepared as described in Example 1 from 3-nitrobenzenisothiocyanate (4.0 g) and 3-ethoxybenzenecarbohydrazide (4.1 g). The title compound had the following physical properties: mp 183-185° C.

### Example 79

(5-benzo[3,4-c]1,2,5-oxadiazol-5-yl(1,3,4-thiadiazol-2-yl))(2,3-dichlorophenyl)amine

The title compound was prepared as described in Example 1 from 3-methyl-4-bromobenzenisothiocyanate (80 mg) and benzo[c] 1,2,5-oxadiazole-5-carbohydrazide (53 mg).

The title compound had the following physical properties: mp 229-231° C.

### Example 80

(3-bromophenyl)[5-(3-nitrophenyl)(1,3,4-thiadiazol-2-yl)]amine

The title compound was prepared as described in Example 1 from 3-bromobenzenisothiocyanate (1.3 g) and 3-nitrobenzenecarbohydrazide (1.0 g). The title compound had the following physical properties: mp 273-275° C.; LC-MS 376.88.

### Example 81

[5-([2-[(4,5-dichloroimidazolyl)methyl]phenoxy]methyl)(1,3,4-thiadiazol-2-yl)](4-bromo-3-chlorophenyl)amine

Following the procedure described in Example 1, the title compound was prepared from 4-bromo-3-chlorobenzenisothiocyanate (430 mg) and 2-{2-[(4,5-dichloroimidazolylthio)methyl]phenoxy}acetohydrazide (500 mg). The title compound had the following physical properties: mp 183-185° C.

### Example 82

[(4-methylphenyl)sulfonyl](4-{[5-(3-nitrophenyl)(1,3,4-thiadiazol-2-yl)]amino}phenyl) amine

Following the procedure described in Example 10 (with sulfonyl chloride in place of acyl chloride), the title compound was prepared from (4-{[5-(3-nitrophenyl)(1,3,4-thiadiazol-2-yl)]amino}phenyl)amine (100 mg) and 4-toluenesulfonyl chloride (96 mg). The title compound had the following physical properties: mp 255-257° C.

### Example 83

(4-{[5(3-ethoxyphenyl)(1,3,4-thiadiazol-2-yl)]aminophenyl)-(phenylsuffonyl)amine

As described in Example 10 (with sulfonyl chloride in place of acyl chloride), the title compound was prepared from (4-{[5-(3-ethoxyphenyl)(1,3,4-thiadiazol-2-yl)]amino}phenyl) amine (100 mg) and benzenesulfonyl chloride (100 mg). The title compound had the following physical properties: mp 216-218° C.

### Example 84

[(4-{[5(3-Nitrophenyl)-1,3,4-thiadiazol-2-yl]amine}phenyl)sulfonyl]piperidine

The reactions described in Example 1 were repeated using 3-nitrobenzenecarbohydrazide (362 mg; 2.0 mmol) and 4-(piperidylsulfonyl) benzenisothiocyanate (565 mg; 2.0 mmol) to yield (3-nitrophenyl)-N-[({[4-(piperidylsulfonyl)phenyl]amino}thioxomethyl)amino]carboxamide (910 mg, 98%) in the first step. In the second step, (3-nitrophenyl)-N-[({[4-(piperidylsulfonyl)phenyl]amino}thioxomethyl)amino]carboxamide (800 mg; 1.7 mmol) and sulfuric acid (2.0 mL) were used to yield the title compound (751 mg; 98%) with the following physical properties: mp 266-267° C.; Mass (M+1)⁺446 (Calc.); 446 (Obsd.). (A&A)

### Example 85

(4-Bromo-3-chlorophenyl)[5-(3-nitrophenyl)(1,3,4-thiadiazol-2-yl)amine

The reactions described in Example 1 were repeated using 3-nitrobenzenecarbohydrazide (362 mg; 2.0 mmol) and 4-bromo-3-chlorobenzenisothiocyanate (497 mg; 2.0 mmol) to yield N-({[(4-bromo-3-chlorophenyl)amino]thioxo-methyl}amino)(3-nitrophenyl)carboxamide (810 mg; 94%) in the first step. In the second step, N-({[(4-bromo-3-chlorophenyl)amino]thioxomethyl}amino)(3-nitrophenyl)carboxamide (750 mg; 1.7 mmol) and sulfuric acid (2.0 mL) were used to yield the title compound (698 mg; 97%) with the following physical properties: mp 330-331° C.; Mass (M+1)⁺413 (Calc.); 413 (Obsd.). (A&A)

### Example 86

[5-(3-Nitrophenyl)(1,3,4-thiadiazol-2-yl) (2,3,4,5-tetrachlorophenyl)amine

The reactions described in Example 1 were repeated using 3-nitrobenzenecarbohydrazide (362 mg; 2.0 mmol) and 2,3,4,5-tetrachlorobenzenisothiocyanate (546 mg; 2.0 mmol) to yield (3-nitrophenyl)-N-({[(2,3,4,5-tetrachlorophenyl)amino]thioxomethyl}amino) carboxamide (690 mg; 76%) in the first step. In the second step, (3-nitrophenyl)-N-({[(2,3,4,5-tetrachlorophenyl)amino]thioxomethyl}amino) carboxamide (550 mg; 1.2 mmol) and sulfuric acid (2.0 mL) were used to yield the title compound (516 mg; 98%) with the following physical properties: mp 250-251° C.; Mass (M+1)⁺437 (Calc.); 437 (Obsd.). (A&A)

### Example 87

(3-Chloro-4-methylphenyl)[5-(3-nitrophenyl)(1,3,4-thiadiazol-2-yl)amine

The reactions described in Example 1 were repeated using 3-nitrobenzenecarbohydrazide (362 mg; 2.0 mmol) and 3-chloro-4-methylbenzenisothiocyanate (367 mg; 2.0 mmol) to yield N-({[(3-chloro-4-methylphenyl)amino]thioxomethyl}amino)-(3-nitrophenyl)carboxamide (700 mg; 96%) in the first step. In the second step, N-({[(3-chloro-4-methylphenyl)amino]thioxomethyl}amino)(3-nitrophenyl)carboxamide (550 mg; 1.5 mmol) and sulfuric acid (2.0 mL) were used to yield the title compound (486 mg; 93%) with the following physical properties: mp 289-290° C.; Mass (M)⁺347 (Calc.); 347 (Obsd.); Elemental analysis C, 51.95; H, 3.20; N, 16.16, S 9.25 (Calc.); C52.12, H 3.16, N 16.16, S 9.42 (Obsd.). (NuMega)

### Example 88

(4-Methylthiophenyl)[5-(3-nitrophenyl)(1,3,4-thiadiazol-2-yl)amine

The reactions described in Example 1 were repeated using 3-nitrobenzenecarbohydrazide (362 mg; 2.0 mmol) and 4-methylthiobenzenisothiocyanate (362 mg; 2.0 mmol) to yield N-({[(4-methylthiophenyl)amino]thioxomethyl}amino)(3-nitrophenyl)carboxamide (710 mg; 98%) in the first step. In the second step, N-({[(4-methylthiophenyl)aminolthioxomethyl}amino)(3-nitrophenyl)carboxamide (600 mg; 1.7 mmol) and sulfuric acid (2.0 mL) were used to yield the title compound (528 mg; 93%) with the following physical properties: mp 247-248° C.; Mass (M+1)⁺345 (Calc.); 345 (Obsd.). (NuMega)

### Example 89

[4-Methylethyl)phenyl][5-(3-nitrophenyl)(1,3,4-thiadiazol-2-yl)amine

The reactions described in Example 1 were repeated using 3-nitrobenzenecarbohydrazide (362 mg; 2.0 mmol) and 4-(methylethyl)benzenisothiocyanate (354 mg; 2.0 mmol) to yield N-({[(4-(methylethyl)phenyl)amino]thioxomethylgamino)-(3-nitrophenyl)carboxamide (680 mg; 94%) in the first step. In the second step, N-({[(4-(methylethyl)phenyl)amino]thioxomethyl}amino)-(3-nitrophenyl)carboxamide (600 mg; 1.7 mmol) and sulfuiric acid (2.0 mL) were used to yield the title compound (516 mg; 90%) with the following physical properties: mp 243-244° C.; Mass (M+1)⁺341 (Calc.); 341 (Obsd.); (M+23)⁺363 (Calc.); 363 (Obsd.). (NuMega)

### Example 90

(4-Butylphenyl)[5-(3-nitrophenyl)(1,3,4-thiadiazol-2-yl)amine

The reactions described in Example 1 were repeated using 3-nitrobenzenecarbohydrazide (362 mg; 2.0 mmol) and 4-butylbenzenisothiocyanate (383 mg; 2.0 mmol) to yield N-({[(4-butylphenyl)amino]thioxomethyl}amino)(3-nitrophenyl)carboxamide (680 mg; 92%) in the first step. In the second step, N-({[(4-butylphenyl)amino]thioxomethyl}amino)(3-nitrophenyl)carboxamide (500 mg; 1.3 mmol) and sulfuric acid (2.0 mL) were used to yield the title compound (428 mg; 90%) with the following physical properties: mp 198-199° C.; Mass (M+1)⁺355 (Calc.); 355 (Obsd.). (A&A)

### Example 91

(4-Decylphenyl)[5-(3-nitrophenyl)(1,3,4-thiadiazol-2-yl)amine

The reactions described in Example 1 were repeated using 3-nitrobenzenecarbohydrazide (154 mg; 0.8 mmol) and 4-decylbenzenisothiocyanate (237 mg; 0.8 mmol) to yield N-({[(4-decylphenyl)amino]thioxomethyl}amino)-(3-nitrophenyl)carboxamide (340 mg; 87%) in the first step. In the second step, N-({[(4-decylphenyl)amino]thioxomethyl}amino)-(3-nitrophenyl)carboxamide (300 mg; 0.7 mmol) and sulfuric acid (2.0 mL) were used to yield the title compound (264 mg; 92%) with the following physical properties: mp 169-171° C.; Mass (M+1)⁺440 (Calc.); 440 (Obsd.). (A&A)

### Example 92

[5-(3-Nitrophenyl)(1,3,4-thiadiazol-2-yl)[4-(4-nitrophenoxy)phenyl]amine

The reactions described in Example 1 were repeated using 3-nitrobenzenecarbohydrazide (362 mg; 2.0 mmol) and 4-(4-nitrophenoxy)benzenisothiocyanate (545 mg; 2.0 mmol) to yield (3-nitrophenyl)-N-[({[4-(4-nitrophenoxy)phenyl]amino}thioxomethyl)amino]carboxamide (890 mg; 98%) in the first step. In the second step, (3-nitrophenyl)-N-[({[4-4-nitrophenoxy)phenyl]amino}thioxomethyl)amino]carboxamide (500 mg; 1.1 mmol) and sulfuric acid (2.0 mL) were used to yield the title compound (480 mg; 100%) with the following physical properties: mp 285-286° C.; Mass (M+1)⁺436 (Calc.); 436 (Obsd.). (A&A)

### Example 93

[(4-{[5-(3-Methoxyphenyl}1,3,4-thiadiazol-2-yl]aminelphenyl)sulfonyl]piperidine

The reactions described in Example 1 were repeated using 3-methoxybenzenecarbohydrazide (332 mg; 2.0 mmol) and 4-(piperidylsulfonyl) benzenisothiocyanate (565 mg; 2.0 mmol) to yield (3-methoxyphenyl)-N-[({[4-(piperidylsulfonyl)phenyl]amino}thioxomethyl)amino]carboxamide (775 mg; 86%) in the first step. In the second step, (3-methoxyphenyl)-N-[({[4-(piperidylsulfonyl)phenyl]amino}thioxomethyl)amino]carboxamide (500 mg; 1.1 mmol) and sulfuric acid (2.0 mL) were used to yield the title compound (751 mg; 980%) with the following physical properties: mp 205-206° C.; Mass (+1)+432 (Calc.); 432 (Obsd.). (A&A)

### Example 94

[(4-{[5-(3-Methylphenyl)-1,3,4-thiadiazol-2-yl]amine}phenyl)sulfonyl]piperidine

The reactions described in Example 1 were repeated using 3-nitrobenzenecarbohydrazide (300 mg; 2.0 mmol) and 4-(piperidylsulfonyl) benzenisothiocyanate (565 mg; 2.0 mmol) to yield (3-methylphenyl)-N-[({[4-(piperidylsulfonyl)phenyl]amino}thioxomethyl)amino]carboxamide (757 mg; 88%) in the first step. In the second step, (3-methylphenyl)-N-[({([4-(piperidylsulfonyl)phenyl]amino}thioxomethyl)amino]carboxamide (500 mg; 1.2 mmol) and sulfuric acid (2.0 mL) were used to yield the title compound (751 mg; 98%) with the following physical properties: mp 213-214° C.; Mass (M+1)⁺415 (Calc.); 415 (Obsd.). (A&A)

### Example 95

(5-Chloro-2,4-dimethoxyphenyl)[5-3-nitrophenyl)(1,3,4-thiadiazol-2-yl)]amine

The reactions described in Example 1 were repeated using 3-nitrobenzenecarbohydrazide (362 mg; 2.0 mmol) and 5-chloro-2,4-dimethoxybenzenisothiocyanate (459 mg; 2.0 mmol) to yield N-({[(5-chloro-2,4-dimethoxyphenyl)amino]thioxomethyl}amino)(3-nitrophenyl)carboxamide (796 mg; 97%) in the first step. In the second step, N-({[(5-chloro-2,4-dimethoxyphenyl)amino]thioxomethyl}amino)(3-nitrophenyl)carboxamide (550 mg; 1.3 mmol) and sulfuric acid (2.0 mL) were used to yield the title compound (470 mg; 89%) with the following physical properties: mp 218-219° C.; Mass (M)⁺393 (Calc.); 393 (Obsd.). (A&A)

### Example 96

(3-Chloro-4-methylphenyl){5-[3-(phenylmethoxy)phenyl](1,3,4-thiadiazol-2-yl)}amine

As described in Procedure D, [(3-chloro-4-methylphenyl)amino]hydrazinomethane-1-thione (3.38 g, 78%) was prepared from 3-chloro-4-methylbenzenisothiocyanate (3.67 g; 20 mmol) and hydrazine monohydrate (1.50 g; 30 mmol).

The reactions described in Example 2 were repeated using [(3-chloro-4-methylphenyl)amino]hydrazinomethane-1-thione (216 mg; 1.0 mmol) and 3-(phenylmethoxy)benzaldehyde (212 mg; 1.0 mmol) to yield ({1-aza-2-[3-(phenylmethoxy)phenyl]vinyl}amino)[(3-chloro-4-methylphenyl)amino]methane-1-thione (320 mg; 78%) in the first step. In the second step, ({1-aza-2-[3-(phenylmethoxy)phenyl]vinyl}amino)[(3-chloro-4-methylphenyl)amino]methane-1-thione (205 mg; 0.5 mmol) and iron (III) chloride hexahydrate (405 mg; 1.5 mmol) were used to yield the title compound (80 mg, 39%) with the following physical properties: mp 211-212° C.; Mass (M)⁺408 (Calc.); 408 (Obsd.). (A&A)

### Example 97

(3-Chloro-4-methylphenyl)[5-(4-morpholin-4-yl-3-nitrophenyl)(1,3,4-thiadiazol-2-yl)]amine

The reactions described in Example 2 were repeated using [(3-chloro-4-methylphenyl)amino]hydrazinomethane-1-thione (216 mg; 1.0 mmol) and 4-morpholin-4-yl-3-nitrobenzaldehyde (236 mg; 1.0 mmol) to yield {[1-aza-2-(4-morpholin-4-yl-3-nitrophenyl)vinyl]amino}[(3-chloro-4-methylphenyl)amino]methane-1-thione (330 mg, 77%) in the first step. In the second step, {[1-aza-2-(4-morpholin-4-yl-3-nitrophenyl)vinyl]amino}[(3-chloro-4-methylphenyl)amino]methane-1-thione (217 mg; 0.5 mmol) and iron (III) chloride hexahydrate (405 mg; 1.5 mmol) were used to yield the title compound (83 mg, 38%) with the following physical properties: mp 268-269° C.; Mass (M)⁺432 (Calc.); 432 (Obsd.). (A&A)

### Example 98

2-(3-{5[(3-Chloro-4-methylphenyl)amino]-1,3,4-thiadiazol-2-ylphenoxy)ethan-1-ol

The reactions described in Example 2 were repeated using [(3-chloro-4-methylphenyl)amino]hydrazinomethane-1-thione (216 mg; 1.0 mmol) and 3-(2-hydroxyethoxy)benzaldehyde (166 mg; 1.0 mmol) to yield 2-{3-[2-aza-2-({[(3-chloro-4-methylphenyl)amino]thioxomethyl}amino)vinyl]phenoxy}ethan-1-ol (145 mg, 40%) in the first step. In the second step, 2-{3-[2-aza-2-({[(3-chloro-4-methylphenyl)amino]thioxomethyl}amino)vinyl]phenoxy}ethan-1-ol (104 mg; 0.3 mmol) and iron (I) chloride hexahydrate (243 mg; 0.9 mmol) were used to yield the title compound (42 mg, 23%) with the following physical properties: mp 193-194° C.; Mass (M+1)⁺363 (Calc.); 363 (Obsd.). (A&A)

### Example 99

(3-Chloro-4-methylphenyl){5-[4-(trifluoromethylthio)phenyl](1,3,4-thiadiazol-2-yl)}amine

The reactions described in Example 2 were repeated using [(3-chloro-4-methylphenyl)amino]hydrazinomethane-1-thione (216 mg; 1.0 mmol) and 4-(trifluoromethylthio)benzaldehyde (206 mg; 11.0 mmol) to yield ({1-aza-2-[4-(trifluoromethylthio)phenyl]vinyl}amino)[(3-chloro-4-methylphenyl)amino]methane-1-thione (268 mg, 66%) in the first step. In the second step, ({1-aza-2-[4-(trifluoromethylthio)phenyl]vinyl}amino)[(3-chloro-4-methylphenyl)amino]methane-1-thione (202 mg; 0.5 mmol) and iron (1H) chloride hexahydrate (405 mg; 1.5 mmol) were used to yield the title compound (89 mg, 44%) with the following physical properties: mp 215-216° C.; Mass (M)⁺402 (Calc.); 402 (Obsd.). (A&A)

### Example 100

[(4-{[5-(4-Bromo-3-chlorophenyl)-1,3,4-thiadiazol-2-yl]amine}phenyl)sulfonyl]piperidine

The reactions described in Example 1 were repeated using 1-bromo-2-chlorobenzene-4-carbohydrazide (250 mg; 1.0 mmol) and 4-(piperidylsulfonyl) benzenisothiocyanate (282 mg; 1.0 mmol) to yield (4-bromo-3-chlorophenyl)-N-[({[4-(piperidylsulfonyl)phenyl]amino}thioxomethyl)amino]carboxamide (510 mg, 96%) in the first step. In the second step, (4-bromo-3-chlorophenyl)-N-[({[4-(piperidylsulfonyl)phenyl]amino}thioxomethyl)amino]carboxamide (800 mg; 1.7 mmol) and sulfuric acid (2.0 mL) were used to yield the title compound (394 mg; 90%) with the following physical properties: mp 262-263° C.; Mass (M+1)+515 (Calc.); 515 (Obsd.). (A&A)

### Example 101

[(4-{[5-3-Bromo-4-chlorophenyl)-1,3,4-thiadiazol-2-yl]amine}phenyl)sulfonyl]piperidine

The reactions described in Example 1 were repeated using 2-bromo-1-chlorobenzene-4-carbohydrazide (250 mg; 1.0 mmol) and 4-(piperidylsulfonyl) benzenisothiocyanate (282 mg; 1.0 mmol) to yield (3-bromo-4-chlorophenyl)-N-[({[4-(piperidylsulfonyl)phenyl]amino}thioxomethyl)amino]carboxamide (510 mg; 96%) in the first step. In the second step, (3-bromo-4-chlorophenyl)-N-[({[4-(piperidylsulfonyl)phenyl]amino}thioxomethyl)amino]carboxamide (450 mg; 0.8 mmol) and sulfuric acid (2.0 mL) were used to yield the title compound (412 mg; 95%) with the following physical properties: mp 246-247° C.; Mass (M+1)⁺515 (Calc.); 515 (Obsd.). (A&A)

### Example 102

(3-Chloro-4-methylphenyl){5-[3-trifluoromethoxy)phenyl](1,3,4-thiadiazol-2-yl)}amine

The reactions described in Example 2 were repeated using [(3-chloro-4-methylphenyl)amino]hydrazinomethane-1-thione (216 mg; 1.0 mmol) and 3-(trfluoromethoxy)benzaldehyde (190 mg; 1.0 mmol) to yield ({1-aza-2-[3-(trifluoromethoxy)phenyl]vinyl}amino)[(3-chloro-4-methylphenyl)amino]methane-1-thione (262 mg; 68%) in the first step. In the second step, ({1-aza-2-[3-(trifluoromethoxy)phenyl]vinyl}amino)[(3-chloro-4-methylphenyl)amino]methane-1-thione (194 mg; 0.5 mmol) and iron (1H) chloride hexahydrate (405 mg; 1.5 mmol) were used to yield the title compound (46 mg; 32%) with the following physical properties: mp 180-181° C.; Mass (M)⁺386 (Calc.); 386 (Obsd.). (A&A)

### Example 103

(5-{3-[4-(ter-Butyl)phenoxy]phenyl}(1,3,4-thiadiazol-2-yl))(3-chloro-4-methylphenyl) amine

The reactions described in Example 2 were repeated using [(3-chloro-4-methylphenyl)amino]hydrazinomethane-1-thione (216 mg; 1.0 mmol) and 3-[4-(tert-butyl)phenoxy]benzaldehyde (254 mg; 1.0 mmol) to yield [(1-aza-2-{3-[4-(tert-butyl)phenoxy]phenyl}vinyl)amino][(3-chloro-4-methylphenyl)amino]methane-1-thione (333 mg; 74%) in the first step. In the second step, [(1-aza-2-{3-[4-(tert-butyl)phenoxy]phenyl}vinyl)amino][(3-chloro-4-methylphenyl)amino]methane-1-thione (226 mg; 0.5 mmol) and iron (E) chloride hexahydrate (405 mg; 1.5 mmol) were used to yield the title compound (50 mg, 22%) with the following physical properties: mp 187-189° C.; Mass (M)⁺450 (Calc.); 450 (Obsd.). (A&A)

### Example 104

(3,4-Dichlorophenyl){5-[4-methoxy-3-(phenylmethoxy)phenyl](1,3,4-thiadiazol-2-yl)}amine

The reactions described in Example 2 were repeated using the product from Procedure D (236 mg; 1.0 mmol) and 4-methoxy-3-(phenylmethoxy)benzaldehyde (242 mg; 1.0 mmol) to yield ({1-aza-2-[4-methoxy-3-(phenylmethoxy)phenyl]vinyl}amino)[(3,4-dichlorophenyl)amino]methane-1-thione (439 mg; 95%) in the first step. In the second step, ({1-aza-2-[4-methoxy-3-(phenylmethoxy)phenyl]vinyl}amino)[(3,4-dichlorophenyl)amino]methane-1-thione (322 mg; 0.7 mmol) and iron (m) chloride hexahydrate (568 mg; 2.1 mmol) were used to yield the title compound (127 mg; 40%) with the following physical properties: mp 223-224° C.; Mass (M+1)⁺459 (Calc.); 459 (Obsd.). (A&A)

### Example 105

(3,4-Dichlorophenyl){5[4-(difluoromethoxy)phenyl](1,3,4-thiadiazol-2-yl)}amine

The reactions described in Example 2 were repeated using the product from Procedure D (236 mg; 1.0 mmol) and 4-(difluoromethoxy)benzaldehyde (172 mg; 1.0 mmol) to yield ({1-aza-2-[4-(difluoromethoxy)phenyl]vinyl}amino)[(3-chloro-4-methylphenyl)amino]methane-1-thione (332 mg, 85%) in the first step. In the second step, ({1-aza-2-[4-(difluoromethoxy)phenyl]vinyl}amino)[(3-chloro-4-methylphenyl)amino]methane-1-thione (234 mg; 0.6 mmol) and iron (m) chloride hexahydrate (486 mg; 1.8 mmol) were used to yield the title compound (134 mg; 58%) with the following physical properties: mp 265-266° C.; Mass (M)⁺388 (Calc.); 388 (Obsd.). (A&A)

### Example 106

(3,4-Dichlorophenyl)[5-(4-butoxyphenyl)(1,3,4-thiadiazol-2-yl)]amine

The reactions described in Example 2 were repeated using the product from Procedure D (236 mg; 1.0 mmol) and 4-butoxybenzaldehyde (178 mg; 1.0 mmol) to yield {[1-aza-2-(3-butoxyphenyl)vinyl]amino}[(3,4-ichlorophenyl)amino]methane-1-thione (303 mg; 76%) in the first step. In the second step, {[1-aza-2-(3-butoxyphenyl)vinyl]amino}[(3,4-dichlorophenyl)amino]methane-1-thione (238 mg; 0.6 mmol) and iron ([) chloride hexahydrate (486 mg; 1.8 mmol) were used to yield the title compound (112 mg; 47%) with the following physical properties: mp 233-235° C.; Mass (M)+394 (Calc.); 394 (Obsd.).

### Example 107

2-(4-{5-[(3,4-Dichlorophenyl)amino](1,3,4-thiadiazol-2-yl)}phenoxy)-1-(4-methylpiperidyl)ethan-1-one

The reactions described in Example 2 were repeated using the product from Procedure D (2.36 g; 10.0 mmol) and 2-(3-formylphenoxy)acetic acid (1.80 g; 10.0 mmol) to yield 2-{3-[2-aza-2-({[(3,4-dichlorophenyl)amino]thioxomethyl}amino)vinyl]phenoxy}acetic acid (4.05 g; 95%) in the first step. In the second step, 2-{3-[2-aza-2-({[(3,4-dichlorophenyl)amino]thioxomethyl}amino)vinyl]phenoxy}acetic acid (3.38 g; 7.9 mmol) and iron (III) chloride hexahydrate (6.43 g; 25.0 mmol) were used to yield ethyl 2-(3-{5-[(3,4-dichlorophenyl)amino]-1,3;4-thiadiazol-2-yl}phenoxy)acetate (2.16 g; 64%).

A solution of 4-methylpiperidine (1.98 g; 20 mmol) and ethyl 2-(3-{5-[(3,4-dichlorophenyl)amino]-1,3,4-thiadiazol-2-yl}phenoxy)acetate (420 mg; 1 mmol) in ethanol (10 mL) was refluxed for 24 hours. After cooling, a white solid was provided (280 mg, 59%): mp 219-220° C.; Mass (M)⁺477 (Calc.); 477 (Obsd.).

### Example 108

(5-{3-[4-tert-Butyl)phenoxy]phenyl}(1,3,4-thiadiazol-2-yl))indan-2-ylamine

As described in Procedure D, hydrazino(indan-2-ylamino)methane-1-thione (2.07 g; 100%) was prepared from indan-2-isothiocyanate (1.75 g; 10.0 mmol) and hydrazine monohydrate (750 mg; 15.0 mmol).

The reactions described in Example 2 were repeated using of hydrazino(indan-2-ylamino)methane-1-thione (415 mg; 2.0 mmol) and 3-[4-(tert-butyl)phenoxy]benzaldehyde (509 mg; 2.0 mmol) to yield [(1-aza-2-{3-[4-(tert-butyl)phenoxy]phenyl}vinyl)amino](indan-2-ylamino)methane-1-thione (491 mg; 55%) in the first step. In the second step, [(1-aza-2-{3-[4-(tert-butyl)phenoxy]phenyl}vinyl)amino](indan-2-ylamino)methane-1-thione (444 mg; 1.0 mmol) and iron (III) chloride hexahydrate (811 mg; 3.0 mmol) were used to yield the title compound (109 mg; 25%) with the following physical properties: mp 132-133° C.; Mass (M)⁺442 (Calc.); 442 (Obsd.).

### Example 109

(5-{3-[4-(tert-Butyl)phenoxy]phenyl}(1,3,4-thiadiazol-2-yl))(3,3,5-trimethylcyclohexyl)amine

As described in Procedure D, hydrazino[(3,3,5-trimethylcyclohexyl)amino]methane-1-thione (1.16 g; 98%) was prepared from 3,3,5-trimethylcyclohexanisothiocyanate (1.01 g; 5.5 mmol) and hydrazine monohydrate (400 mg; 8.0 mmol).

The reactions described in Example 2 were repeated using hydrazino[(3,3,5-trimethylcyclohexyl)amino]methane-1-thione (431 mg; 2.0 mmol) and 3-[4-(tert-butyl)phenoxy]benzaldehyde (509 mg; 2.0 mmol) to yield [(1-aza-2-{3-[4-(tert-butyl)phenoxy]phenyl}vinyl)amino][(3,3,5-trimethylcyclohexyl)amino]methane-1-thione (484 mg; 54%) in the first step. In the second step, [(1-aza-2-{3-[4-(tert-butyl)phenoxy]phenyl}vinyl)amino][(3,3,5-trimethylcyclohexyl)amino]methane-1-thione (452 mg; 11.0 mmol) and iron (III) chloride hexahydrate (811 mg; 3.0 mmol) were used to yield the title compound (240 mg, 53%) with the following physical properties: mp 166-167° C.; Mass (M)⁺450 (Calc.); 450 (Obsd.).

### Example 110

(5-{3-[4-(tert-Butyl)phenoxy]phenyl}(1,3,4-thiadiazol-2-yl))(methylhexyl)amine

As described in Procedure D, hydrazino[(methylhexyl)amino]methane-1-thione (1.81 g; 96%) was prepared from heptan-2-isothiocyanate (1.57 g; 10.0 mmol) and of hydrazine monohydrate (750 mg; 15.0 mmol).

The reactions described in Example 2 were repeated using hydrazino[(methylhexyl)amino]methane-1-thione (379 mg; 2.0 mmol) and 3-[4-(tert-butyl)phenoxy]benzaldehyde (509 mg; 2.0 mmol) to yield [(1-aza-2-{3-[4-(tert-butyl)phenoxy]phenyl}vinyl)amino][(methylhexyl)amino]methane-1-thione (320 mg, 78%) in the first step. In the second step, [(1-aza-2-{3-[4-(tert-butyl)phenoxy]phenyl}vinyl)amino][(methylhexyl)amino]methane-1-thione (425 mg; 1.0 mmol) and iron (III) chloride hexahydrate (811 mg; 3.0 mmol) were used to yield the title compound (211 mg; 50%) with the following physical properties: mp 115-116° C.; Mass (M)⁺424 (Calc.); 424 (Obsd.).

### Example 111

(5-{3-[3-(Trifluoromethyl)phenoxy]phenyl}(1,3,4-thiadiazol-2-yl))(3,3,5-trimethylcyclohexyl) amine

The reactions described in Example 2 were repeated using hydrazino[(3,3,5-trimethylcyclohexyl)amino]methane-1-thione (431 mg; 2.0 mmol) and 3-[3-(trifluoromethyl)phenoxy]benzaldehyde (532 mg; 2.0 mmol) to yield [(1-aza-2-{3-[3-(trifluoromethyl)phenoxy]phenyl}vinyl)amino][(3,3,5-trimethylcyclohexyl)amino]methane-1-thione (420 mg; 45%) in the first step. In the second step, [(1-aza-2-{3-[3-(trifluor-omethyl)phenoxy]phenyl}vinyl)amino][(3,3,5-triethylcyclohexyl)amino]methane-1-thione (400 mg; 0.9 mmol) and iron (III) chloride hexahydrate (700 mg; 2.6 mmol) were used to yield the title compound (121 mg; 30%) with the following physical properties: mp 101-102° C.; Mass (M)⁺462 (Calc.); 462 (Obsd.).

### Example 112

2-[4-(henylmethoxy)phenyl]-N-(5-{3-[3-(trifluoromethyl)phenoxy]phenyl}(1,3,4-thiadiazol-2-yl))acetamide

The reactions described in Example 10 were repeated using the product from Example 23 (202 mg; 0.6 mmol), 2-[4-(phenylmethoxy)phenyl]acetyl chloride (313 mg; 1.2 mmol) and dimethyl-4-pyridylamine (244 mg; 2.0 mmol) to yield the title compound (107 mg; 32%) with the following physical properties: mp 194-195° C.; Mass (M)⁺562 (Calc.); 562 (Obsd.).

### Example 113

Naphthyl-N-(5-{3-[3-(trifluoromethyl)phenoxy]phenyl}(1,3,4-thiadiazol-2-yl))carboxamide

The reactions described in Example 10 were repeated using the product from Example 23 (202 mg; 0.6 mmol), naphthalene-2-carbonyl chloride (229 mg; 1.2 mmol) and dimethyl-4-pyridylamine (244 mg; 2.0 mmol) to yield the title compound (241 mg; 82%) with the following physical properties: mp 236-238° C.; Mass (M+1)+492 (Calc.); 492 (Obsd.).

### Example 114

2-[(3-{5-[(3,4-Dichlorophenyl)amino](1,3,4-thiadiazol-2-yl)}phenyl)[3-(trifluoromethyl)phenyl]methoxy]acetic acid.

The reactions described in Example 7 were repeated using ethyl 2-[(3-{5-[(3,4-dichlorophenyl)amino](1,3,4-thiadiazol-2-yl)}phenyl)[3-(trifluoromethyl)phenyl]methoxy]acetate (210 mg; 0.4 mmol), lithium hydroxide (72 mg; 3 mmol), MeOH/H₂O (3:1) (5 mL) and THF (3 mL) to yield the title compound (55 mg; 28%) with the following physical properties: mp 89-91° C.; Mass (M)⁺554 (Calc.); 554 (Obsd.).

### Example 115

2-[4-(tert-Butyl)phenoxy]-N-(5-{3-[3-(trifluoromethyl)phenoxy]phenyl}(1,3,4-thiadiazol-2-yl))acetamide

The reactions described in Example 10 were repeated using the product from Example 23 (202 mg; 0.6 mmol), 2-[4-(tert-butyl)phenoxy]acetyl chloride (181 mg; 0.8 mmol) and dimethyl-4-pyridylamine (244 mg; 2.0 mmol) to yield the title compound (110 mg; 35%) with the following physical properties: mp 179-180° C.; Mass (M)+528 (Calc.); 528 (Obsd.).

### Example 116

[2-(4-Methoxyphenoxy)-5-nitrophenyl]-N-(5-{3-[3-(trifluoromethyl)phenoxy]phenyl}(1,3,4-thiadiazol-2-yl))carboxamide

The reactions described in Example 10 were repeated using the product from Example. 23 (202 mg; 0.6 mmol), 2-(4-methoxyphenoxy)-5-nitrobenzoyl chloride (246 mg; 0.8 mmol) and dimethyl-4-pyridylamine (244 mg; 2.0 mmol) to yield the title compound (15 mg; 4%) with the following physical properties: mp 130-132° C.; Mass)⁺609 (Calc.); 609 (Obsd.).

### Example 117

[5-(3,5-Dichlorophenloxy)(2-furyl)]-N-(5-{3-[3-(trifluoromethyl)phenoxy]phenyl}(1,3,4-thiadiazol-2-yl))carboxamide

The reactions described in Example 10 were repeated using the product from Example 23 (202 mg; 0.6 mmol), 5-(3,5-dichlorophenoxy)furan-2-arbonyl chloride (233 mg; 0.8 mmol) and dimethyl-4-pyridylamine (244 mg; 2.0 mmol) to yield the title compound (30 mg; 8%) with the following physical properties: mp 205-207° C.; Mass (M)+592 (Calc.); 592 (Obsd.).

### Example 118

(3-Nitrophenyl)-N-(5-3-[3-(trilluoromethyl)phenoxy]phenyl)(1,3,4-thiadiazol-2-yl))carboxamide

The reactions described in Example 10 were repeated using the product from Example 23 (202 mg; 0.6 mmol), 3-nitrobenzoyl chloride (148 mg; 0.8 mmol) and dimethyl-4-pyridylamine (244 mg; 2.0 mmol) to yield the title compound (72 mg; 25%) with the following physical properties: mp 200-202° C.; Mass (M+1)+487 (Calc.); 487 (Obsd.).

### Example 119-120

3-{2-[aza(4-bromo-3-chlorophenyl)methylene]-3-benzyl(1,3,4-thiadiazoln-5yl))-1-phenoxybenzene

and

(4-bromo-3-chlorophenyl)benzyl[5-(3-phenoxyphenyl)(1,3,4-thiadiazol-2-yl)]amine

The reaction described in Example 8 was repeated using (4-bromo-3-chlorophenyl)[5-(3-phenoxyphenyl)(1,3,4-thiadiazol-2-yl)]amine (126 mg; 0.27 mmol), potassium tert-butoxide (0.27 mL; 0.27 mmol) and benzyl bromide (0.039 mL; 0.33 mmol) to yield 3-{2-[aza(4-bromo-3-chlorophenyl)methylene]-3-benzyl(1,3,4-thiadiazolin-5-yl)}-1-phenoxybenzene (21 mg; 19%) with the following physical properties: Rf: 0.56 (hexanes/ethyl acetate, 2/1); MS (M)⁺: 547, 549, 551.

¹H NMR (300 MHz, di-CDCl₃): δ 7.53 (1H, d), 7.45 (2H, d), 7.38-7.30 (8H, m), 7.18-7.11 (2H, m), 7.02-6.70 (3H, m), 6.85 (1H, dd), 5.31 (2H, s), and

(4-bromo-3-chlorophenyl)benzyl[5-(3-phenoxyphenyl)(1,3,4-thiadiazol-2-yl)]amine (44 mg; 30%) with the following physical properties: R_{f}: 0.45 (hexanes/ethyl acetate, 2/1); MS (M)⁺: 547, 549, 551;

¹H NMR (300 MHz, di-CDCl₃): δ 7.59 (1H, d), 7.46-7.44 (3H, m), 7.37-7.28 (8H, m), 7.15-7.01 (5H, m), 5.20 (2H, s).

### Example 121

2-{5-[(4-bromo-3-chlorophenyl)amino]-1,3,4-thiadiazol-2-yl}-3-hydroquinazolin-4-one

The reactions described in Example 1 were repeated using 4-oxo-3-hydroquinazoline-2-carbohydrazide (100 mg; 0.49 mmol) and 4-bromo-3-chlorobenzenisothiocyanate (122 mg; 0.49 mmol) to yield N-({[(4-bromo-3-chlorophenyl)amino] thioxomethyl}amino)(4-oxo(3-hydroquinazolin-2-yl))carboxamide in the first step. In the second step, all the crude product and sulfuric acid (0.4 mL) were used to yield the title compound (85 mg; 40%) with the following physical properties: mp 348-349° C.; MS (M)⁺: 433, 435, 437.

### Example 122

(5-benzo[3,4-c]1,2,5-oxadiazol-5-yl(1,3,4-thiadiazol-2-yl))(4-bromo-3-chlorophenyl)amine

The reactions described in Example 1 were repeated using benzo[c]1,2,5-oxadiazole-5-carbohydrazide (150 mg; 0.84 mmol) and 4-bromo-3-chlorobenzenisothiocyanate (209 mg; 0.49 mmol) to yield benzo[3,4-c]1,2,5-oxadiazol-5-yl-N-({[(4-bromo-3-chlorophenyl)amino]thioxomethyl}amino)carboxamide in the first step. In the second step, all the crude product and sulfuric acid (0.5 mL) were used to yield the title compound (272 mg; 79%) with the following physical properties: mp 326-327° C.; Anal. Calcd for C₁₄H₇BrClN₅OS: C, 41.15; H, 1.73; N, 17.14; S, 7.85. Found: C, 41.28; H, 1.62; N, 16.93; S, 8.01.

### Example 123

ethyl 4-{[5-(4-phenoxyphenyl)-1,3,4-thiadiazol-2-yl]amino}benzoate

The reactions described in Example 1 were repeated using 1-phenoxybenzene-4-carbohydrazide (500 mg; 2.2 mmol) and ethyl 4-isothiocyanatobenzoate (454 mg; 2.2 mmol) to yield ethyl 4-[({[(4-phenoxyphenyl)carbonylamino]amino}thioxomethyl)amino]benzoate in the first step. In the second step, all the crude product and sulfuric acid (0.5 mL) were used to yield the title compound (589 mg; 64%) with the following physical properties: mp 206-207° C.; MS (M+H)⁺: 418.

### Example 124

(3,4-dichlorophenyl)[5-(3-{[4-(tert-butl)phenyl]methoxy}phenyl)(1,3,4-thiadiazol-2-yl)]{[4-(tert-butyl)phenyl]methyl}amine

The reaction described in Example 8 was repeated using 3-{5-[(3,4-dichlorophenyl)amino]-1,3,4-thiadiazol-2-yl}phenol (200 mg; 0.59 mmol), potassium tert-butoxide (1.18 mL; 1.18 mmol) and 1-(tert-butyl)-4-(bromomethyl)benzene (0.1 mL; 0.59 mmol) to yield the title compound (168 mg; 45%) with the following physical properties:

¹H NMR (300 MHz, di-CDCl₃): δ 7.50-7.18 (14H, m), 7.04-7.00 (1H, m), 5.19 (2H, s), 5.06 (2H, s), 1.33 (9H, s), 1.29 (9H, s).

### Example 125

5-{5-[(3,4-dichlorophenyl)amino](1,3,4-thiadiazol-2-yl)}-3-[3-trifluoromethyl)phenoxy]phenol

The reactions described in Procedure B were followed using methyl 3,5-dihydroxybenzoate (5.00 g; 29.7 mmol), 1-bromo-3-(trifluoromethyl)benzene (6.2 mL; 44.5 mmol), potassium carbonate (8.22 g; 59.5 mmol), copper(II) oxide (4.73 g; 59.5 mmol) and pyridine (30 mL) to yield methyl 5-hydroxy-3-[3-(trifluoromethyl)phenoxy]benzoate (1.22 g; 13%) as white solid.

¹H NMR (300 MHz, di-CDCl₃): δ 7.49-7.37 (3H, m), 7.26-7.17 (3H, m), 6.74 (1H, s), 6.10 (1H, br s), 3.90 (3H, s).

A solution of methyl 5-hydroxy-3-[3-(trifluoromethyl)phenoxy]benzoate (1.22 g; 3.9 mmol) and hydrazine monohydrate (0.38 mL; 7.8 mmol) in dry ethanol (4.5 mL) under nitrogen was refluxed for 30 hours. After cooling to RT, the solvent was removed by rotovapor. The residue was wash by water then hexanes, dried by vacuum to yield 5-hydroxy-3-[3-(trifluoromethyl)phenoxy]benzenecarbohydrazide (980 mg; 80%) as a light yellow solid.

The reactions described in Example 1 were repeated using 5-hydroxy-3-[3-(trifluoromethyl)phenoxy]benzenecarbohydrazide (500 mg; 1.6 mmol) and 3,4-dichlorobenzenisothiocyanate (327 mg; 1.6 mmol) to yield N-({[(3,4-dichlorophenyl)amino]thioxomethyl}amino) {3-[3-(trifluoromethyl)phenoxy]phenyl}carboxamide in the first step. In the second step, all the crude product and sulfuric acid (0.8 mL) were used to yield the title compound (712 mg; 89%) with the following physical properties: mp 193-194° C.; MS (M)⁺: 497, 499.

### Example 126

(3,4-dichlorophenyl){5-[3-(3-nitrophenoxy)phenyl](1,3,4-thiadiazol-2-yl)}amine

The reactions described in Procedure B were followed using (3-hydroxyphenyl) formaldehyde (5.00 g; 40.9 mmol), 1-bromo-3-nitrobenzene (9.925 g; 49.1 mmol), potassium carbonate (11.318 g; 81.9 mmol), copper(II) oxide (6.513 g; 81.9 mmol) and pyridine (50 mL) to yield [3-(3-nitrophenoxy)phenyl]formaldehyde (2.49 g; 25%) as light yellow solid.

¹H NMR (300 MHz, d₁-CDCl₃): δ 10.01 (1H, s), 8.01 (1H, dd), 7.83 (1H, t), 7.73 (1H, d), 7.63-7.52 (3H, m), 7.39-7.33 (2H, m).

The reactions described in Example 2 were repeated using [3-(3-nitrophenoxy)phenyl]formaldehyde (2.240 g; 9.2 mmol) and the product from Procedure D (2.177 g; 9.2 mmol) to yield ({(1E)-1-aza-2-[3-(3-nitrophenoxy)phenyl]vinyl}amino)[(3,4-dichlorophenyl)amino]methane-1-thione in the first step. In the second step, all the crude product and iron(II) chloride hexahydrate (7.47 g; 27.6 mmol) were used to yield the title compound (3.71 g; 88%) with the following physical properties: mp 210.5-211.5° C.; MS (M)⁺: 458, 460.

### Example 127

2-[3-(3-{5-[(3,4-dichlorophenyl)amino]-1,3,4-thiadiazol-2-yl}phenoxy)phenyl]acetic acid

The reactions described in Procedure B were followed using (3-bromophenyl)formaldehyde (2.95 g; 15.96 mmol), methyl 2-(3-hydroxyphenyl)acetate (2.56 g; 15.96 mmol), potassium carbonate (4.41 g; 31.93 mmol), copper(II) oxide (2.54 g; 31.93 mmol) and pyridine (20 mL) to yield methyl 2-[3-(3-carbonylphenoxy)phenyl]acetate (532 mg; 12%) as a colorless oil.

¹H NMR (300 MHz, d₁-CDCl₃): δ 9.96 (1H, s), 7.61 (1H, d), 7.53-7.47 (2H, m), 7.35-7.26 (2H, m), 7.08 (1H, d), 6.99 (1H, s), 6.94 (1H, d), 3.70 (3H, s), 3.62 (2H, s).

The reactions described in Example 2 were repeated using methyl 2-[3-(3-carbonylphenoxy)phenyl]acetate (532 mg; 1.97 mmol) and the product from Procedure D (465 mg; 1.97 mmol) to yield methyl 2-(3-{3-[(1E)-2-aza-2-({[(3,4-dichlorophenyl)amino]thioxomethyl}amino)vinyl]phenoxy}phenyl)acetate in the first step. In the second step, all the crude product and iron(III) chloride hexahydrate (1.597 g; 5.9 mmol) were used to yield methyl 2-[3-(3-{5-[(3,4-dichlorophenyl)amino-1,3,4-thiadiazol-2-yl}phenoxy)phenyl]acetate (491 mg; 51%).

The reaction described in Example 7 was repeated using methyl 2-[3-(3-{5-[(3,4-dichlorophenyl)amino]-1,3,4-thiadiazol-2-yl}phenoxy)phenyl]acetate (140 mg; 0.29 mmol) and lithium hydroxide (20 mL; 0.25______) to yield the title compound (123 mg; 91%) with the following physical properties: mp 183-184° C.; MS (M)⁺: 471, 473.

### Example 128

4-(3-{-[(3,4-dichlorophenyl)amino]-1,3,4-thiadiazol-2-yl}phenoxy)phenol

The reactions described in Procedure B were followed using 4-hydroxyphenyl benzoate (3.00 g; 14.0 mmol), (3-bromophenyl)formaldehyde (3.3 mL; 28.0 mmol), potassium carbonate (3.87 g; 28.0 mmol), copper(II) oxide (2.23 g; 28.0 mmol) and pyridine (15 mL) to yield 4-(3-carbonylphenoxy)phenyl benzoate (305 mg; 9%) and [3-(4-hydroxyphenoxy)phenyl]formaldehyde (417 mg; 14%).

The reactions described in Example 2 were repeated using [3-(4-hydroxyphenoxy) phenyl]formaldehyde (306 mg; 1.4 mmol) and the product from Procedure D (337 mg; 1.4 mmol) to yield 4-{3-[(1E)-2-aza-2-({[(3,4-dichlorophenyl)amino]thioxomethyl}amino)vinyl]phenoxy}phenol in the first step. In the second step, all the crude product and iron (M) chloride hexahydrate (1.159 g; 4.3 mmol) were used to yield the title compound (332 mg; 55%) with the following physical properties: mp 224-225.5° C.; MS (M)⁺: 429, 431.

### Example 129

(3,4-dichlorophenyl)[5-(3-{[3-(trifluoromethoxy)phenyl]methoxy}phenyl)(1,3,4-thiadiazol-2-yl)]amine

To a solution of 3-{5-[(3,4-dichlorophenyl)amino]-1,3,4-thiadiazol-2-yl}phenol (150 mg; 0.44 mmol) in dry DMF (5 mL) was added a solution of potassium tert-butoxide (0.44 mL, 1M, 0.44 mmol) in THF at RT under an argon atmosphere. After 5 minutes, [3-(bromomethyl)phenoxy]trifluoromethane (0.086 mL; 0.53 mmol) was injected, and the solution was stirred overnight. The DMF was evaporated (rotovap) under vacuum. The crude residue was purified by flash chromatography (ethyl acetate/hexanes, 1:10 to 1:2) to yield the title compound (138 mg; 61%) with the following physical properties: mp 166-167° C.; MS (M)⁺: 511, 5.13.

### Example 130

(4-bromo-3-chlorophenyl) [5-(4-ethoxyphenyl)(1,3,4-thiadiazol-2-yl)]amine

The reactions descried in Example 1 were repeated using 4-ethoxybenzenecarbohydrazide (145 mg; 0.80 mmol) and 4-bromo-3-chlorobenzenisothiocyanate (200 mg; 0.80 mmol) to yield N-({[(4-bromo-3-chlorophenyl)amino]thioxomethyl}amino)(4-ethoxyphenyl)carboxamide (314 mg; 92%) in the first step. In the second step, all the crude product and sulfuric acid (0.2 mL) were used to yield the title compound (205 mg; 68%) with the following physical properties: mp 225-226° C.;

¹H NMR (300 MHz, d₆-DMSO): δ 10.78 (1H, s), 8.13 (1H, d), 7.78 (2H, d), 7.71 (1H, d), 7.43 (1H, dd), 7.06 (2H, d), 4.10 (2H, q), 1.35 (3H, t).

### Example 131-132

(3-bromophenyl)[5-(3-nitropheny)(1,3,4-thiadiazol-2-yl)]benzylamine and

2-[aza(3-bromophenyl)methylene]-5-3-nitrophenyl)-3-benzyl-1,3,4-thiadiazoline

The reaction described in Example 8 was repeated using (3-bromophenyl)[5-(3-nitrophenyl)(1,3,4-thiadiazol-2-yl)]amine (150 mg; 0.40 mmol), potassium tert-butoxide (0.40 mL; 0.40 mmol) and benzyl bromide (0.057 mL; 0.47 mmol) to yield 2-[aza(3-bromophenyl)methylene]-5-(3-nitrophenyl)-3-benzyl-1,3,4-thiadiazoline (46 mg; 24%) with the following physical properties: R_{f}: 0.50 hexanes/ethyl acetate, 2/1); mp 133-134° C.;

¹H NMR (300 MHz, d₁-CDCl₃): δ 8.49 (1H, s), 8.25 (1H, d), 7.93 (1H,d), 7.60 (1H, t), 7.51 (2H, d), 7.41-7.33 (3H, m), 7.26-7.23 (3H, m), 7.06-7.03 (1H, m), 5.40 (2H,s); and (3-bromophenyl)[5-(3-nitrophenyl)(1,3,4-thiadiazol-2-yl))benzylamine (136 mg; 73%) with the following physical properties: R_{f}: 0.35 (hexanes/ethyl acetate, 2/1); MS (M)⁺: 466, 468;

¹H NMR (300 MHz, d₁-CDCl₃): δ 8.53 (1H, s), 8.24 (1H, d), 8.18 (1H, d), 7.60 (1H, t), 7.51-7.47 (2H, m), 7.34-7.23 (7H,m), 5.26 (2H, s).

### Example 133

[5-(3,4-dimnethoxyphenyl)(1,3,4-thiadiazol-2-yl)](4-bromo-3-chlorophenyl)amine

The reactions described in Example 1 were repeated using 1,2-dimethoxybenzene-4-carbohydrazide (158 mg; 0.80 mmol) and 4-bromo-3-chlorobenzenisothiocyanate (200 mg; 0.80 mmol) to yield (3,4-dimethoxyphenyl)-N-({[(4-bromo-3-chlorophenyl)amino]thioxomethyl}amino)carboxamide (330 mg; 93%) in the first step. In the second step, all the crude product and sulfuric acid (0.3 mL) were used to yield the title compound (218 mg; 70%) with the following physical properties: mp 251.5-252.5° C.;

¹H NMR (300 MHz, d₆-DMSO): δ 10.81 (1H, s), 8.14 (1H, d), 7.72 (1H, d), 7.45-7.35 (3H, m), 7.07 (1H, d).

### Example 134

4-{5-[(4-bromo-3-chlorophenyl)amino]-1,3,4-thiadiazol-2-yl}benzene-1,2-diol

The reactions described in Example 1 were repeated using 2H-benzo[d]1,3-dioxolane-5-carbohydrazide (145 mg; 0.80 mmol) and 4-bromo-3-chlorobenzenisothiocyanate (200 mg; 0.80 mmol) to yield 2H-benzo[3,4-d]1,3-dioxolan-5-yl-N-({[(4-bromo-3-chlorophenyl)amino]thioxomethyl}amino)carboxamide (302 mg; 88%) in the first step with the following physical properties:

¹H NMR (300 MHz, di-CDCl₃): δ 10.43 (1H, s), 9.95 (1H, s), 9.87 (1H, s), 7.83 (1H, s), 7.71 (1H, d), 7.54 (1H, d), 7.48-7.46 (2H, m), 7.04 (1H, d), 6.12 (2H, s).

In the second step, all the crude product and sulfuric acid (0.4 mL) were used to yield the title compound (190 mg; 68%) with the following physical properties: mp 220° C. decomposed; ¹H NMR (300 MHz d₆-DMSO): δ 10.73 (1H, s), 9.56 (1H, s), 9.42 (1H, s), 8.13 (1H, d), 7.71 (1H, d), 7.41 (1H, dd), 7.30 (1H, d), 7.11 (1H, dd), 6.83 (1H, d).

### Example 135

(4-bromo-3-chlorophenyl)[5-3-phenoxyphenyl)(1,3,4-thiadiazol-2-yl)]amine

The reactions described in Example 1 were repeated using 1-phenoxybenzene-3-carbohydrazide (184 mg; 0.80 mmol) and 4-bromo-3-chlorobenzenisothiocyanate (200 mg; 0.80 mmol) to yield N-({[(4-bromo-3-chlorophenyl)amino]thioxomethyl amino)(3-phenoxyphenyl)carboxamide (365 mg; 95%) of in the first step. In the second step, all the crude product and sulfuric acid (0.4 mL) were used to yield the title compound (286 mg; 81%) with the following physical properties: mp 216-217° C.; MS (M)⁺: 457, 459, 461.

### Example 136

(4-{5-[(4-bromo-3-chlorophenyl)amino](1,3,4-thiadiazol-2-yl)}phenyl)diethylamine

The reactions described in Example 1 were repeated using 4-(diethylamino) benzenecarbohydrazide (167 mg; 0.80 mmol) and 4-bromo-3-chlorobenzenisothiocyanate (200 mg; 0.80 mmol) to yield [4-(diethylamino)phenyl]-N-({[(4-bromo-3-chlorophenyl) amino]thioxomethyl}amino)carboxamide (319 mg; 88%) in the first step. In the second step, all the crude product and sulfuric acid (0.4 mL) were used to yield the title compound (226 mg; 74%) with the following physical properties: mp 232-233° C.;

¹H NMR (300 MHz, d₆-DMSO): δ 10.68 (1H, s), 8.13 (1H, d), 7.70 (1H, d), 7.63 (2H, d), 7.40 (1H, dd), 6.74 (2H, d), 3.40 (4H, q), 1.12 (6H, t).

### Example 137

(4-bromo-3-chlorophenyl)[5-(3-methylphenyl)(1,3,4-thiadiazol-2-yl)]amine

The reactions described in Example 1 were repeated using 1-methylbenzene-3-carbohydrazide (167 mg; 0.80 mmol) and 4-bromo-3-chlorobenzenisothiocyanate (121 mg; 0.80 mmol) to yield N-({[(4-bromo-3-chlorophenyl)amino]thioxomethyl}amino)(3-methylphenyl)carboxamide (306 mg; 93%) in the first step. In the second step, all the crude product and sulfuric acid (0.3 mL) were used to yield the title compound (230 mg; 79%) with the following physical properties: mp 230-231° C.;

¹H NMR (300 MHz, d₆-DMSO): δ 10.87 (1H, s), 8.14 (1H, s), 7.74-7.66 (3H, m), 7.46-7.38 (2H, m), 7.33 (1H, d), 2.39 (3H, s).

### Example 138

(naphthylmethyl)[5-(3-nitrophenyl)(1,3,4-thiadiazol-2-yl)]amine

The reactions described in Example 1 were repeated using 1-nitrobenzene-3-carbohydrazide (245 mg; 1.35 mmol) and naphthylmethanisothiocyanate (270 mg; 1.35 mmol) to yield N-({[(naphthylmethyl)amino]thioxomethyl}amino)(3-nitrophenyl)carboxamide (482 mg; 94%) in the first step. In the second step, all the crude product and sulfuric acid (0.5 mL) were used to yield the title compound (244 mg; 53%) with the following physical properties: mp 150-151° C.;

¹H NMR (300 MHz, d₆-DMSO): δ 8.72 (1H, t), 8.51 (1H, s), 8.27 (1H, d), 8.18 (1H, d), 8.14 (1H, d), 7.99 (1H, d), 7.91 (1H, d), 7.77 (1H, t), 7.64-7.48 (4H, m), 5.05 (2H, d).

### Example 139

(3,4-dichlorophenyl)[5-(4-phenylphenyl)(1,3,4-thiadiazol-2-yl)]amine

The reactions described in Example 1 were repeated using 1-phenylbenzene-4-carbohydrazide (200 mg; 0.94 mmol) and 3,4-dichlorobenzenisothiocyanate (192 mg; 0.94 mmol) to yield N-({[(3,4-dichlorophenyl)amino]thioxomethyl}amino)(4-phenylphenyl)carboxamide (360 mg; 92%) in the first step. In the second step, all the crude product and sulfuric acid (0.4 mL) were used to yield the title compound (283 mg; 82%) with the following physical properties: mp 305.5-306.5° C.;

¹H NMR (300 MHz, d₆-DMSO): δ 10.92 (1H, s), 8.16 (1H, d), 7.97 (2H, d), 7.84 (2H, d), 7.74 (2H, d), 7.62 (1H, d), 7.54-7.49 (3H, m), 7.45-7.42 (1H, m).

### Example 140

(4-{5[(3,4-dichlorophenyl)amino](1,3,4-thiadiazol-2-yl}lphenyl)dimethylamine

The reactions described in Example 1 were repeated using 4-(dimethylamino) benzenecarbohydrazide (200 mg; 1.1 mmol) and 3,4-dichlorobenzenisothiocyanate (228 mg; 1.1 mmol) to yield [4-(dimethylamino)phenyl]-N-({[(3,4-dichlorophenyl)amino]thioxomethyl}amino)carboxamide (404 mg; 94%) in the first step. In the second step, all the crude product and sulfuric acid (0.4 mL) were used to yield the title compound (368 mg; 96%) with the following physical properties: mp 298.5-299.5° C.;

¹HNMR (300 MHz, d₆-DMSO): δ 10.70 (1H, s), 8.13 (1H, s), 7.66 (2H, d), 7.58 (1H, d), 7.48 (1H, d), 6.79 (2H, d), 2.99 (6H, s).

### Example 141

(3,4-dichlorophenyl)[5-(4-methylthiophenyl)(1,3,4-thiadiazol-2-yl)]amine The reactions described in Example 1 were repeated using 1-methylthiobenzene-4-carbohydrazide (200 mg; 1.1 mmol) and 3,4-dichlorobenzenisothiocyanate (224 mg; 1.1 mmol) to yield N-({[(3,4-dichlorophenyl)amino]thioxomethyl}amino)(4-methylthiophenyl)carboxamide (358 mg; 84%) in the first step. In the second step, all the crude product and sulfuric acid (0.4 mL) were used to yield the title compound (251 mg; 74%) with the following physical properties: mp 247.5-248.5° C.;

¹H NMR (300 MHz, d₆-DMSO): δ 10.86 (1H, s), 8.13 (1H, d), 7.80 (2H, d), 7.60 (1H, d), 7.49 (1H, dd), 7.38 (2H, d), 2.54 (3H, s).

### Example 142

methyl 3-({2-[aza(3,4-dichlorophenyl)methylene]-5-(3-ethoxyphenyl)-1,3,4-thiadiazolin-3-yl}methyl)benzoate

The reactions described in Example 1 were repeated using 1-ethoxybenzene-3-carbohydrazide (1.000 g; 5.6 mmol) and 3,4-dichlorobenzenisothiocyanate (1.133 g; 5.6 mmol) to yield N-({[(3,4-dichlorophenyl)amino]thioxomethyl}lamino)(3-ethoxyphenyl)carboxamide (1.922 g; 90%) in the first step. In the second step, all the crude product and sulfuric acid (2.0 mL) were used to yield the title compound (1.738 g; 95%) with the following physical properties:

¹H NMR (300 MHz, d₆-DMSO): δ 10.88 (1H, s), 8.15 (1H, d), 7.61 (1H, d), 7.51 (1H, dd), 7.44-7.39 (3H, m), 7.10-7.07 (1H, m), 4.11 (2H, q), 1.37 (3H, t).

### Example 143-144

methyl 3-({2-[aza(3,4-dichlorophenyl)methylene]-5-(3-ethoxyphenyl)-1,3,4-thiadiazolin-3-yl}methyl)benzoate

and

methyl 3-({(3,4-dichlorophenyl)[5-(3-ethoxyphenyl)(1,3,4-thiadiazol-2-yl)]amino}methyl)benzoate

The reaction described in Example 8 was repeated using (3,4-dichlorophenyl)[5-(3-ethoxyphenyl)(1,3,4-thiadiazol-2-yl)]amine (120 mg; 0.33 mmol), potassium tert-butoxide (33 mL; 0.33 mmol) and methyl 3-(bromomethyl)benzoate (90 mg; 0.39 mmol) to yield methyl 3-({2-[aza(3,4-dichlorophenyl)methylene]-5-(3-ethoxyphenyl)-1,3,4-thiadiazolin-3-yl}methyl)benzoate (24 mg; 14%) with the following physical-properties: R_{f}: 0.55 (hexanes/ethyl acetate, 2/1); mp 87-88° C.; MS (M)⁺: 513, 515; and methyl 3-({(3,4-dichlorophenyl)[5-(3-ethoxyphenyl)(1,3,4-thiadiazol-2-yl)]amino}methyl)benzoate (128 mg; 75%) with the following physical properties: R_{f}: 0.34 (hexanes/ethyl acetate, 2/1); MS (M)⁺: 513, 515.

### Example 145

(3,4-dichlorophenyl)[5-(3-ethoxyphenyl)(1,3,4-thiadiazol-2-yl)][(4-phenylphenyl)methyl]amineethyl)benzoate

The reaction described in Example 8 was repeated using (3,4-dichlorophenyl)[5-(3-ethoxyphenyl)(1,3,4-thiadiazol-2-yl)]amine (120 mg; 0.33 mmol), potassium tert-butoxide (0.33 mL; 0.33 mmol) and 4-(bromomethyl)-1-phenylbenzene (97 mg; 0.39 mmol) to yield the title compound (132 mg; 75%) with the following physical properties: MS (M+H)⁺: 532, 534;

¹H NMR (300 MHz, d₁-CDCl₃): δ 7.58-7.26 (14H, m), 7.21 (1H, dd), 6.96-6.91 (1H, m), 5.26 (2H, s), 4.07 (2H, q), 1.42 (3H, t).

### Example 146

1-({2-[aza(3,4-dichlorophenyl)methylene]-5(3-ethoxyphenyl)(1,3,4-thiadiazolin-3-yl)}methyl)-3-methoxybenzene

The reaction described in Example 8 was repeated using (3,4-dichlorophenyl)[5-(3-ethoxyphenyl)(1,3,4-thiadiazol-2-yl)]amine (120 mg; 0.33 mmol), potassium tert-butoxide (0.33 mL; 0.33 mmol) and 3-(bromomethyl)-1-methoxybenzene (79 mg; 0.39 mmol) to yield 1-({2-[aza(3,4-dichlorophenyl)methylene]-5-(3-ethoxyphenyl)(1,3,4-thiadiazolin-3-yl)}methyl)-3-methoxybenzene (21 mg; 13%) with the following physical properties: R_{f}: 0.54 (hexanes/ethyl acetate, 2/1); mp 89-90° C.; MS (M)⁺: 485, 487; and (3,4-dichlorophenyl)[5-(3-ethoxyphenyl)(1,3,4-thiadiazol-2-yl)][(3-methoxyphenyl)methyl]amine (108 mg; 67%) with the following physical properties: R_{f}: 0.38 (hexanes/ethyl acetate, 2/1); MS (M)⁺: 485, 487.

### Example 147

(3,4-dichlorophenyl)[5-(3-ethoxyphenyl)(1,3,4-thiadiazol-2-yl)][(3-nitrophenyl)methyl]amine

The reaction described in Example 8 was repeated using (3,4-dichlorophenyl)[5-(3-ethoxyphenyl)(1,3,4-thiadiazol-2-yl)]amine (120 mg; 0.33 mmol), potassium tert-butoxide (0.33 mL; 0.33 mmol) and 3-(bromomethyl)-1-nitrobenzene (85 mg; 0.39 mmol) to yield 3-{2-[aza(3,4-dichlorophenyl)methylene]-3-[(3-nitrophenyl)methyl](1,3,4-thiadiazolin-5-yl)}-1-ethoxybenzene (30 mg; 18%) with the following physical properties: R_{f}: 0.53 (hexanes/ethyl acetate, 2/1); mp 114-115° C.; MS (M)⁺: 500, 502; and (3,4-dichlorophenyl)[5-(3-ethoxyphenyl)(1,3,4-thiadiazol-2-yl)][(3-nitrophenyl)methyl]amine (98 mg; 59%) with the following physical properties: R_{f}: 0.30 (hexanes/ethyl acetate, 2/1); MS (M)⁺: 500, 502;

¹H NMR (300 MHz, di-CDCl₃): δ 8.18 (1H, s), 8.16 (1H, d), 7.78 (1H, d), 7.55-7.49 (3H, m), 7.38 (1H, s), 7.32-7.19 (3H, m), 6.95 (1H, d), 5.33 (2H, s), 4.07 (2H, q), 1.42 (3H, t).

### Example 148-149

3-{2-[aza(3,4-dichlorophenyl)methylene]-3-(2-naphthylmethyl)(1,3,4-thiadiazolin-5-yl)-1-ethoxybenzene

and

(3,4-dichlorophenyl)[5-3-ethoxyphenyl)(1,3,4-thiadiazol-2-yl)](2-naphthylmethyl)amine

The reaction described in Example 8 was repeated, but using of (3,4-dichlorophenyl)[5-(3-ethoxyphenyl)(1,3,4-thiadiazol-2-yl)]amine (120 mg; 0.33 mmol), potassium tert-butoxide (0.33 mL; 0.33 mmol) and 2-(bromomethyl)naphthalene (87 mg; 0.39 mmol) to yield 3-{2-[aza(3,4-dichlorophenyl)methylene]-3-(2-naphthylmethyl)(1,3,4-thiadiazolin-5-yl)}-1-ethoxybenzene (23 mg; 14%) with the following physical properties: R_{f}: 0.52 (hexanes/ethyl acetate, 2/1); mp 74-75° C.; MS (M)⁺: 505, 507; and (3,4-dichlorophenyl)[5-(3-ethoxyphenyl)(1,3,4-thiadiazol-2-yl)](2-naphthylmethyl)amine (110 mg; 66%) with the following physical properties: R_{f}: 0.43 (hexanes/ethyl acetate, 2/1); mp 43-44° C.; MS (M)⁺: 505, 507.

### Example 150

(3,4-dichlorophenyl)(5-[3-(4-methoxyphenoxy)phenyl](1,3,4-thiadiazol-2-yl)}amine

The reactions described in Example 2 were repeated, but using [3-(4-methoxyphenoxy)phenyl]formaldehyde (193 mg; 0.85 mmol) and the product from Procedure D (200 mg; 0.85 mmol) to yield ({(1E)-1-aza-2-[3-(4-methoxyphenoxy)phenyl]vinyl}amino)[(3,4-dichlorophenyl)amino]methane-1-thione (326 mg; 86%) in the first step. In the second step, all the crude product and iron (m) chloride hexahydrate (593 mg; 2.20 mmol) were used to yield the title compound (158 mg; 45/o) with the following physical properties: mp 198-199° C.; MS (M)⁺: 443, 445.

### Example 151

(3,4-dichlorophenyl){5-[3-(4-methylphenoxy)phenyl](1,3,4-thiadiazol-2-yl)}amine

The reactions described in Example 2 were repeated using [3-(4-methylphenoxy)phenyl]formaldehyde (180 mg; 0.85 mmol) and the product from Procedure D (200 mg; 0.85 mmol) to yield ({(1E)-1-aza-2-[3-(4-methylphenoxy)phenyl]vinyl}amino)[(3,4-dichlorophenyl)amino]methane-1-thione (322 mg; 88%) in the first step. In the second step, all the crude product and of iron (III) chloride hexahydrate (607 mg; 2.25 mmol) were used to yield the title compound (166 mg; 52%) with the following physical properties: mp 210-211° C.; MS (M)⁺: 427, 429.

### Example 152

(3,4-dichlorophenyl){5-[3-(3,5-dichlorophenoxy)phenyl](1,3,4-thiadiazol-2-yl)}amine

The reactions described in Example 2 were repeated using [3-(3,5-dichlorophenoxy) phenyl]formaldehyde (226 mg; 0.85 mmol) and the product from Procedure D (200 mg; 0.85 mmol) to yield ({(1E)-1-aza-2-[3-(3,5-dichlorophenoxy)phenyl]vinyl}amino)[(3,4-dichlorophenyl)amino]methane-1-thione (366 mg; 89%) in the first step. In the second step, all the crude product and iron (11 chloride hexahydrate (612 mg; 2.26 mmol) were used to yield the title compound (192 mg; 53%) with the following physical properties: mp 242-243° C.; MS M)⁺: 481, 483, 485.

### Example 153

(3,4-dichlorophenyl)(5-[3-(3,4-dichlorophenoxy)phenyl](1,3,4-thiadiazol-2-yl)}amine

The reactions described in Example 2 were repeated using [3-(3,4-dichlorophenoxy)phenyl]formaldehyde (226 mg; 0.85 mmol) and the product from Procedure D (200 mg; 0.85 mmol) to yield ({(1E)-1-aza-2-[3-(3,4-dichlorophenoxy)phenylvinyl}amino)[(3,4-dichlorophenyl)amino]methane-1-thione (292 mg; 71%) in the first step. In the second step, all the crude product and iron (I) chloride hexahydrate (488 mg; 1.81 mmol) were used to yield the title compound (20 mg; 7%) with the following physical properties: mp 189-190° C.; MS (M)⁺: 481, 483, 485.

### Example 154

(3,4-dichlorophenyl)(5-{3-[3-(trifluoromethyl)phenoxy]phenyl}(1,3,4-thiadiazol-2-yl))amine

The reactions described in Example 2 were repeated using {3-[3-(trifluoromethyl)phenoxy]phenyl}formaldehyde (226 mg; 0.85 mmol) and the product from Procedure D (200 mg; 0.85 mmol) to yield [((1E)-1-aza-2-{3-[3-(trifluoromethyl)phenoxy]phenylvinyl)amino][(3,4-dichlorophenyl)amino]methane-1-thione (282 mg; 69%) in the first step. In the second step, all the crude product and iron (III) chloride hexahydrate (472 mg; 1.75 mmol) were used to yield the title compound (156 mg; 56%) with the following physical properties: mp 212-213° C.; MS (M)⁺: 481, 483.

### Example 155

(5-(2H-benzo[d]1,3-dioxolan-5-yl)(1,3,4-thiadiazol-2-yl))(3,4-dichlorophenyl)amine

The reactions described in Example 2 were repeated using 2H-benzo[3,4-d]1,3-dioxolan-5-ylformaldehyde (127 mg; 0.85 mmol) and the product from Procedure D (200 mg; 0.85 mmol) to yield [((1E)-2-(2H-benzo[3,4-d]1,3-dioxolan-5-yl)-1-azavinyl)amino][(3,4-dichlorophenyl) amino]methane-1-thione (274 mg; 88%) in the first step. In the second step, all the crude product and iron (m) chloride hexahydrate (604 mg; 2.23 mmol) were used to yield the title compound (120 mg; 50%) with the following physical properties: mp 271-272° C.;

¹H NMR (300 MHz, d₆-DMSO): δ 10.82 (1H, s), 8.13 (1H, d), 7.59 (1H, d), 7.49 (1H, dd), 7.44 (1H, s), 7.36 (1H, d), 7.05 (1H, d), 6.13 (2H, s).

### Example 156

Methyl 4-{[(3,4-dichlorophenyl)(5-{3-[3-trifluoromethyl)phenoxy]phenyl}(1,3,4-thiadiazol-2-yl))amino]methyl}benzoate

As described in Example 8, the title compound was prepared from (3,4-dichlorophenyl)(5-{3-[3-(trifluoromethyl)-phenoxy]phenyl}(1,3,4-thiadiazol-2-yl))amine (300 mg) and methyl-(4-(bromomethyl)benzoate (210 mg). The title compound had the following physical properties: MS 629.96.

### Example 157

4-{[(3,4-dichlorophenyl)(5-{3-[3-(trifluoromethyl)phenoxy]phenyl}-(1,3,4-thiadiazol-2-yl))amino]methyl}benzoic acid

As described in Example 7, the title compound was prepared from methyl 4-{[(3,4-dichlorophenyl)(5-{3-[3-(trifluoromethyl)phenoxy]phenyl}(1,3,4-thiadiazol-2-yl))amino]methyl}benzoate (50 mg) and sodium hydroxide (0.8 mL; 2.5M). The title compound had the following physical properties: mp 114-116° C. (from 4/1 Hexanes/Ethyl Acetate), MS 615.93.

### Example 158

(3,4-dichlorophenyl)(5-{3-[3-(oxymethyl)phenoxy]phenyl)(1,3,4-thiadiazol-2-yl))amine

The reaction described in Procedure B was repeated using 3-hydroxybenzyl alcohol (1.5 g) and 3-bromobenzaldehyde (2.7 g). In the second step, using a procedure similar to that described in Example 2, {3-[3-hydroxymethyl)phenoxy]phenyl}formaldehyde (500 mg), semicarbazide (400 mg) and FeCl₃6H₂O (120 mg) in ethanol (10 mL) were used to yield the title compound with the following physical properties: mp 128-130° C., MS 443.90.

### Example 159

[(4-methylphenyl)sulfonyl](4-{[5-(3-nitrophenyl)(1,3,4-thiadiazol-2-yl)]amino}phenyl)amine

As described in Example 1, (4-aminophenyl)[5-(3-nitrophenyl)(1,3,4-thiadiazol-2-yl)]amine was prepared from (tert-butoxy)-N-(4-isothiocyanatophenyl)carboxamide (200 mg) and N-amino(3-nitrophenyl)-carboxamide (130 mg). The Boc protecting group is lost during the cyclization reaction.

As described in Example 10 (with sulfonyl chloride in place of acyl chloride), the title compound was prepared from (4-{[5-(3-nitrophenyl)(1,3,4-thiadiazol-2-yl)]amino}phenyl)amine (100 mg) and 4-toluenesulfonyl chloride (96 mg). The title compound had the following physical properties: mp 255-257° C.

### Example 160

(3,4-dichlorophenyl){5-[3,5-bis(phenylmethoxy)phenyl](1,3,4-thiadiazol-2-yl)}amine

As described in Example 2, the title compound was prepared from [3,5-bis(phenylnethoxy)phenyl]formaldehyde (400 mg) and [(3,4-dichloro-phenyl)amino]hydrazinomethane-1-thione (290 mg). The title compound had the following physical properties: mp 212-214° C.; LC-MS: 533.99.

### Example 161

3-{5-[(3-bromophenyl)amino]-1,3,4-thiadiazol-2-yl}benzoic acid

As described in Example 2, the title compound was prepared from 3-carbonylbenzoic acid (70 mg) and [(3-bromophenyl)amino]hydrazinomethane-1-thione (100 mg). The title compound had the following physical properties: mp 270-272° C.

### Example 162

(5-benzo[c]1,2,5-oxadiazol-5-yl(1,3,4-thiadiazol-2-yl))(3,4-dichlorophenyl)amine

As described in Example 1, the title compound was prepared from 3,4-dichlorobenzenisothiocyanate (120 mg) and N-aminobenzo[3,4-c]1,2,5-oxadiazol-5-ylcarboxamide (88 mg). The title compound had the following physical properties: mp 315-317° C.; LC-MS 364.1.

### Example 163

[(3-phenylmethoxy)phenyl](5-{3-[3-trifluoromethyl)phenoxy]-phenyl)(1,3,4-thiadiazol-2-yl)}amine

As described in Example 2, the title compound was prepared from {3-[3-(trifluoromethyl)phenoxy]phenyl}formaldehyde (490 mg) and [({3-phenylmethoxy}phenyl)amino]hydrazinomethane-1-thione (500 mg). The title compound had the following physical properties: mp 150-152° C.; LC-MS: 520.02.

### Example 164

3-{3-[5-(3,4-Dichloro-phenylimino)-4-methyl-4,5-dihydro-[1,3,4]thiadiazol-2-yl-phenoxy}-benzoic acid

3-{3-[5-(3,4-Dichloro-phenylimino)-4-methyl4,5-dihydro-[1,3,4]thiadiazol-2-yl]-phenoxy}-benzoic acid methyl ester: The title compound was prepared using the procedure described in Example 8 from 3-{3-[5-(3,4-Dichloro-phenylamino)-[1,3,4]thiadiazol-2-yl]-phenoxy}-benzoic acid methyl ester (0.32 mmols, 150 mg), potassium tert-butoxide (0.32 mL, 1 M, 0.32 mmol) and methyl iodide (3.2 mmol, 0.2 mL). Yield: 54 mg (350/0). ¹H NMR (300 MHz, d₆-DMSO): δ 7.78-7.07 (11H, m), 3.84 (3H, s), 3.69 (3H, s).

The title compound was prepared using the procedure described in Example 7 from 3-{3-[5-(3,4-Dichloro-phenylimino)-4-methyl-4,5-dihydro-[1,3,4]thiadiazol-2-yl)-phenoxy)-benzoic acid methyl ester (0.03 mmols, 16 mg) and LiOH (0.03 mmol, 7.8 mg). Yield: 12 mg (85%). mp 193-196° C.

¹H NMR (300 MHz, d₁-CDCl₃): δ 7.89 (1H, d), 7.70 (1H, s), 7.50-7.21 (7H, m), 7.07 (1H, d), 6.95 (1H, d), 3.78 (3H, s).

### Example 165

4-{N′-[1-(3-{5-[3-(3-Trifluoromethyl-phenoxy)-phenyl]-[1,3,4]thiadiazol-2-ylamino}-phenyl)-ethylidene]-hydrazino}-benzoic acid

N-({[(3-acetylphenyl)amino]thioxomethyl}amino){3-[3(trifluoromethyl)phenoxy]phenyl}carboxamide: The title compound was prepared using the procedure described in Example 1 from 3-(3-Trifluoromethyl-phenoxy)-benzoic acid hydrazide (1 mmol, 300 mg) and 1-(3-Isothiocyanato-phenyl)-ethanone (1 mmol, 180 mg).

1-(3-{5-[3-(3-Trifluoromethyl-phenoxy)-phenyl]-[1,3,4]thiadiazol-2-ylamino}-phenyl)ethanone was prepared using the procedure described in Example 1 from N-({[(3-acetylphenyl)amino]thioxomethyl}amino) {3-[3(trifluoromethyl)phenoxy] phenyl}carboxamide and sulfuric acid (0.4 mL). Yield: 158 mg (35% from first step). mp 130-133° C.

¹H NMR (300 MHz, d, —CDCl₃): δ 9.67 (1H, s), 8.09 (1H, s), 7.75-7.20 (10H, in), 7.10 (1H, d), 2.65 (3H, s).

A solution of 1-(3-{5-[3-(3-Trifluoromethyl-phenoxy)-phenyl]-[1,3,4]thiadiazol-2-ylamino}-phenyl)-ethanone (0.18 mmol, 80 mg) and 4-Hydrazino-benzoic acid (0.18 mmol, 27 mg) in dry ethanol (5 mL) under nitrogen refluxed overnight. After cooling to room temperature, the mixture was filtered and the solid washed by ethanol, 1N HCl (aqueous), water and hexanes to give the title compound as a light brown solid (60 mg, 57%); mp 277.5-279° C. LCMS (M+H) 589.9; (M−H) 588.0.

### Example 166

4-({[5-(3-phenoxyphenyl)-1,3,4-thiadiazol-2-yl]amino}methyl)benzoic acid

A solution of the product from Procedure D (3-phenoxybenzaldehyde) (1 g, 5.04 mmol) and methyl 4-(isocyanate)benzoate (1.04 g, 5.04 mmol) in dry Ethanol (25 mL) under Argon was refluxed for two hours. After cooling to room temperature, the mixture was filtered and the solid washed by ethanol.

The solid was suspended in dry ethanol (20 mL) and iron (III) chloride hexahydrate (5.7 mmol, 1.54 g) was added. The reaction mixture was refluxed for two more hours, then cooled to room temperature. The solid was collected by filter and washed by ethanol, then crystallized from ethyl acetate/hexane yielded 1.36 g (65%) of methyl 4-({[5-(3-phenoxyphenyl)-1,3,4-thiadiazol-2-yl]amino}methyl)benzoate.

A solution of methyl 4-({[5-(3-phenoxyphenyl)-1,3,4-thiadiazol-2-ylamino}methyl)benzoate (1.36 g, 3.26 mmol) and LiOH (1.96 g, 3.5 mmol) in 10 mL of a mixture of H₂O/MeOH/THF (6:2:2) was stirred at room temperature for two hours. The solvent was removed and the residue was purified using flash chromatography on silica gel. Eluting with chloroform/methanol (91:10) provide a white solid (1.16 g, 850%) of 4-({[5-(3-phenoxyphenyl)-1,3,4-thiadiazol-2-yl]amino}methyl)benzoic acid.

MS (SSQ 7000): M+=404. Column: Betasil C18 5 μm 50×3 mm Solvent A: Water+0.01% AcOH; Solvent B: CAN+0.1% AcOH M.P: 192-195° C.

### Example 167

N-[4-({(3,4-dichlorophenyl)[5-(3-phenoxyphenyl) (1,3,4-thiadiazol-2-yl)]amino}methyl)phenyl]methanecarboxylic acid.

Following the procedure described in Example 8, the methyl ester of the title compound was prepared from 0.17 g of (3,4-dichlorophenyl)[5-(3-phenoxyphenyl)(1,3,4-thiadiazol-2-yl)]amine and 0.11 g of methyl {N-[4-(bromomethyl)phenyl]carbamoyl}-formate.

This ester was subjected to the procedure described in Example 7, yielding the title compound from 0.11 g of methyl {N-[4-({(3,4-dichlorophenyl)[5-(3-phenoxyphenyl)(1,3,4-thiadiazol-2-yl)]amino}methyl)phenyl]carbamoyl}formate and 2.0 mL of 2.5M lithium hydroxide, with the following physical properties: mp 198-200° C. (from 2/1 Hexanes/Ethyl Acetate), LC-MS 589.

### Example 168

4-({(3,4-dichlorophenyl)[4-(4-phenylphenyl)(1,3-thiazol-2-yl)]amino}methyl)benzoic acid.

Following the procedure described in Example 8, the methyl ester of the title compound was prepared from 0.40 g of (3,4-dichlorophenyl)[4-(4-phenylphenyl)(1,3-thiazol-2-yl)]amine and 0.23 g of methyl-(4-(bromomethyl)benzoate.

This ester was subjected to the procedure described in Example 7, yielding the title compound from 0.30 g of methyl 4-({(3,4-dichlorophenyl)[4-(4-phenylphenyl)(1,3-thiazol-2-yl)]amino}methyl)benzoate and 2.0 mL of 2.5 M lithium hydroxide, with the following physical properties: mp 202-204° C. (from 4/1 Hexanes/Ethyl Acetate), LC-MS 529.

### Example 169

N-(3-([(3,4-dichlorophenyl)(5-{3-[3(trifluoromethyl)phenoxy}phenyl)-(1,3,4-thiadiazol-2-yl))amino]methyl}phenyl)methanecarboxylic acid

Following the procedure described in Example 8, the methyl ester of the title compound was prepared from 0.3 g of (3,4-dichlorophenyl)(5-{3-[3-(trifluoromethyl)phenoxy]phenyl}(1,3,4-thiadiazol-2-yl))amine and 0.2 g of methyl {N-[4-(bromomethyl)phenyl]carbamoyl}formate.

This ester was subjected to the procedure described in Example 7, yielding the title compound from 0.11 g of methyl[N-(4-{[(3,4-dichlorophenyl)(5-{3-[3-(trifluoromethyl)phenoxy]phenyl}(1,3,4-thiadiazol-2-yl))amino]methyl}phenyl)carbamoyl]formate and 0.2 mL 2.5M lithium hydroxide, with the following physical properties: mp 172-174° C. (from 4/1 Hexanes/Ethyl Acetate), MS 659.4 [M+].

### Example 170

2-{4-[(2-[aza(3,4-dichlorophenyl)methylene]-5-{3-[3-(trifluoromethyl)phenoxy]phenyl}-1,3,4-thiadiazolin-3-yl)methyl]phenyl}acetic acid.

Following the procedure described in Example 8, the methyl ester of the title compound was prepared from 0.30 g of (3,4-dichlorophenyl)(5-{3-[3-(trifluoromethyl)phenoxy]phenyl}(1,3,4-thiadiazol-2-yl))amine and 0.15 g of methyl-(4-(bromomethyl)benzoate.

This ester was subjected to the procedure described in Example 7, yielding the title compound from 0.013 g of methyl 2-(4-{[(3,4-dichlorophenyl)(5-{3-[3-(trifluoromethyl)phenoxyphenyl}(1,3,4-thiadiazol-2-yl))amino]methyl}phenyl)acetate and 0.04 mL of 2.5M lithium hydroxide, with the following physical properties: mp 136-138° C. (from 4/1 Hexanes/Ethyl Acetate), MS 630.5 [M+].

### Example 171

4-{5-[3-3-{5-[(3,4-dichlorophenyl)amino]-1,3,4-thiadiazol-2-yl}phenoxy) phenyl]-1,2,3,4-tetrazoyl}butanoic acid

3-(3-{5-[3,4-dichlorophenylamino]-1,3,4-thiadiazol-2-yl}phenoxy) benzenecarbonitrile, 3.2 mmol, was dissolved in dry DMF. Sodium azide, 9.5 mmol, and ammonium chloride, 9.5 mmol, were added and the mixture was stirred at 115° C. for 24 hours. The mixture was cooled to room temperature and recharged with 5 mmol of sodium azide/ammonium chloride and heated at 115° C. for 6 hours. The mixture was then cooled to room temperature and applied directly to silica gel chromatography, hexane/acetone (4/1) to yield 1.2 g of pure (3,4-Dichlorophenyl)-(5-{3-[3-(2H-tertazol-5-yl)-phenoxy]-phenyl}-[1,3,4]thiadiazol-2-yl)-amine (78%).

(3,4-Dichlorophenyl)-(5-{3-[3-(2H-tertazol-5-yl)-phenoxy]-phenyl}-[1,3,4]thiadiazol-2-yl)-amine, 0.09 mmol, was dissolved in ˜1 mL acetone/DMF (1/1). Triethylamine, 0.12 mmol, and 4-Iodo-methyl butyrate, 0.1 mmol, were added and heated to 80° Celsius for two hours. The residue was applied directly to silica gel chromatography. The pure product was hydrolyzed in lithium hydroxide/THF/Methanol (2/1/1). The solution was then neutralized with 1M HCl (aq). The crude product was collected by filtration, washing several times with water, followed by hexane, then dried en vacuo. The product was recrystallized from ethanol/water to give 25 mg (48%) of the title compound, with the following physical properties: mp 137-142° C. (decomposition); MS (A&A Labs); m/z 568.2/570.2 [M+H].

### Example 172

4-({(1E)-1-aza-2-[3-3-(5[(3,4-dichlorophenyl)amino](1,3,4-thiadiazol-2-yl)}phenoxy)phenyl]vinyl}amino)benzoic acid

3-(3-[1,3]dioxalan-2-yl-phenoxy) benzaldehyde, 16.6 mmol, and [(3,4-dichloropheny)amino]hydrazinomethane-1-thione, 16.6 mmol, were combined in ethanol and heated to reflux for two hours. The solution was cooled to room temperature and the precipitate was collected by filtration, and washed with hexane (3×). The white solid was slurried in ethanol and 3 eq of Fe(III)Cl₃ was added. The slurry was refluxed for two hours, cooled and the crude product collected by filtration. The crude product was washed (3×) with water followed by hexane. The product was recrystallized from hot ethanol/water to give 3.2 g of the title compound. Yield 39%(isolated).

This product was subjected to 0.1M HCl in methanol at room temperature for 18 hours to reveal the aldehyde, 3-{3-[5-(3,4-dichloro-phenylamino)-[1,3,4]thiadiazol-2-yl]-phenoxy}-benzaldehyde, which was purified on silica gel; hexane/ethyl acetate (1/1), followed by recrystalization to give 1.7 g of product (60%).

3-{3-[5-(3,4-dichloro-phenylamino)-[1,3,4]thiadiazol-2-yl]-phenoxy}-benzaldehyde 0.38 mmol, and 4-carboxy-phenyl hydrazine were combined in ethanol and heated at reflux for 4 hours. Upon cooling the product precipitated and was washed with water and hexane to give 75 mg (34%) of analytically pure title compound, with the following physical properties: mp 259-262° C. MS (A&A Labs); m/z 576.5 [M+H].

### Example 173

(6Z)-7-[3-(3-{5-[3,4-dichlorophenyl)amino](1,3,4-thiadiazol-2-yl)}phenoxy)phenyl]heptanoic acid

[6-(Ethoxycarbonyl)hexyl]triphenylphosphonium bromide, 35 mmol, in 75 mL TBF at 0° C. under an Argon atmosphere was treated with 34 mmol of Na HMDS (2.5M in TBF) and stirred for 30 minutes at room temperature. The mixture was then cooled to 0° C. and 3-(3-[1,3] dioxalan-2-yl-phenoxy) benzaldehyde, 3.5 mmol in 10 mL THF was added via syringe. The ice bath was removed and the reaction was stirred at room temperature for one hour. The reaction was then diluted with ether and 250 mL of saturated ammonium chloride was added. The aqueous layer was extracted with ether (3×) and the combined organic fraction was washed with brine, dried with MgSO₄, and the solvent removed on a rotovap. The crude compound was purified by silica gel chromatography (hexane/ethyl acetate) to give 1.2 g of product (−9/1) cis/trans. The acetal was hydrolyzed with 1N HCl/ThF (1/9) at room temp for 12 hours. The product was purified by silica gel chromatography to yield 765 mg of the cis isomer, 7-[3-(3-Formyl-phenoxy)-phenyl]-hept-6-enoic acid ethyl ester (yield: 63%, from 3-(3-[1,3] dioxalan-2-yl-phenoxy) benzaldehyde).

7-[3-(3-Formyl-phenoxy)-phenyl]-hept-6-enoic acid ethyl ester. 0.85 mmol, and [(3,4-dichloropheny)amino]hydrazinomethane-1-thione, 0.85 mmol, were combined as described in Example 2, followed by hydrolysis with 0.25M LiOH in THF to give 200 mg of the title compound, yield: 43%, which was recrystallized from ethanol/water, with the following physical properties: mp 142-145° C. MS m/z 540.3 [M+H].

Selected compounds of the invention are evaluated for biological activity as inhibitors of tyrosine phosphatase as described previously, and the results are presented in FIG. 1.

In addition, selected compounds of the present invention, identified by example or compound number, have been evaluated for biological activity as inhibitors of other tyrosine phosphatases as described previously, and the results are as follows:

| | PTP-1B | TC-PTP | PTP-β | CD-45 |
|---|---|---|---|---|
| Compound | IC₅₀ (μM) | IC₅₀ (μM) | IC₅₀ (μM) | IC₅₀ (μM) |
| Example 16 | 12 | 30, 30 | >100 | 30 |
| Example 135 | 6 | 20, 12 | >100 | 20 |
| Example 66 | 7 | 20, 30 | >100 | 25 |
| Example 80 | 7 | 14, 8 | >100 | 80 |
| Example 14 | 5 | 12 | 24 | 10 |
| Example 7 | 12 | 15 | 31 | 17 |
| Example 172 | 0.8 | 1.6 | 30 | 14 |
| Example 168 | 1 | | | |
| Example 169 | 1 | | | |
| Example 171 | 2 | | | |
| Compound 187 | 1 | 1 | 5 | 5 |
| Compound 188 | 10 | | | |
| Compound 190 | 5 | | | |
| Compound 198 | 4 | | | |

All patents and patent applications cited in this specification are hereby incorporated by reference as if they had been specifically and individually indicated to be incorporated by reference.

1. A method of inhibiting protein tyrosine phosphatase activity which comprises administering to a mammal an effective amount of a compound having the formula: 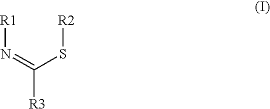
or a pharmaceutically acceptable salt thereof, wherein:
R1, R2, and R3 are each independently selected from
H, hydroxyl, alkoxy, alkylthio, nitro, amino or amido (each unsubstituted or substituted with alkyl, amino, cycloheteroalkyl, cycloalkyl fluoro, aryl, heteroaryl, cycloheteroalkyl, alkylthio, arylthio, cyano, OR′, OC═OR″, C═O—OR′″, or C═O—NR″″R″″);
small alkyl (C1-C10) (unsubstituted or substituted with alkyl, amino, cycloheteroalkyl, cycloalkyl fluoro, aryl, heteroaryl, cycloheteroalkyl, alkylthio, P═O(OR″)₂, CH₂P═O(OR″)₂, CF₂P═O(OR″)₂, NHCOCOOR″, CH(COOR″)₂, arylthio, cyano, OR″, OC═OR″, C═O—OR′″, or C═O—NR″″R″″);
phenyl and mono and disubstituted (at positions 3 and 4) phenyl (wherein the phenyl ring is independently substituted with alkyl, trifluoromethyl, mono and di halogen atoms, alkylthio, alkoxy, nitro, cyano, morphilino, cyclohexyl, phenyl, phenolic, dioxymethylene, nitro, acetylamino, OR′, P═O(OR″)₂, CH₂P═O(OR″)₂, CF₂P═O(OR″)₂, NHCOCOOR″, CH(COOR″)₂);
heteroaryl, cycloheteroalkyl and cycloheteroalkyl (each unsubstituted or substituted with alkyl, halogen, alkylthio, alkoxy, or nitro, OR′, P═O(OR″)₂, CH₂P═O(OR″)₂, CF₂P═O(OR″)₂, NHCOCOOR″, CH(COOR″)₂,);
cycloalkyl (C3-C10) (unsubstituted or substituted with alkyl, fluoro, aryl, heteroaryl, cycloheteroalkyl, cycloheteroalkyl alkylthio, arylthio, cyano, P═O(OR″)₂, CH₂P═O(OR″)₂, CF₂P═O(OR″)₂, NHCOCOOR″, CH(COOR″)₂, OR′, OC═OR′, C═O—OR″, or C═O—NR′″ R″″);
alkenyl (C1-C10) (unsubstituted or substituted with alkyl, fluoro, aryl, heteroaryl, cycloheteroalkyl, cycloheteroalkyl, alkylthio, arylthio, cyano, P═O(OR″)₂, CH₂P═O(OR″)₂, CF₂P═O(OR″)₂, NHCOCOOR″, CH(COOR″)₂, OR′, OC—OR′, C═O—OR″, or C═O—NR′″R″″);
alkadienyl (C1-C10) (unsubstituted or substituted with alkyl, fluoro, aryl, heteroaryl, cycloheteroalkyl, cycloheteroalkyl, alkylthio, arylthio, cyano, P═O(OR″)₂, CH₂P═O(OR″)₂, CF₂P═O(OR″)₂, NHCOCOOR″, CH(COOR″)₂, OR′, —OC═OR5, —C═O—OR″, C═O—NR′″R″″);
cycloalkenyl (C4-C10), unsubstituted or substituted with alkyl, fluoro, aryl, heteroaryl, cycloheteroalkyl, cycloheteroalkyl, alkylthio, arylthio, cyano, P═O(OR″)₂, CH₂P═O(OR″)₂, CF₂P═O(OR″)₂, NHCOCOOR″, CH(COOR″)₂, OR′, —OC═OR′, —C═O—OR″, C═O—NR′″ R″″;
bicycloalkyl (C5-C12), unsubstituted or substituted with alkyl, fluoro, aryl, heteroaryl, cycloheteroalkyl, cycloheteroalkyl, alkylthio, arylthio, cyano, P═O(OR″)₂, CH₂P═O(OR″)₂, CF₂P═O(OR″)₂, NHCOCOOR″, CH(COOR″)₂, OR′, —OC═OR′, —C═O—OR″, C—O—NR′″R″″;
tricycloalkyl (C8-C14), unsubstituted or substituted with alkyl, fluoro, aryl, heteroaryl, cycloheteroalkyl, cycloheteroalkyl, alkylthio, arylthio, cyano, P═O(OR″)₂, CH₂P═O(OR″)₂, CF₂P═O(OR″)₂, NHCOCOOR″, CH(COOR″)₂, OR′, —OC═OR′, —C═O—OR″, C═O—NR′″R″″;
where
Each R′ is independently:
hydrogen;
alkyl (C1-C10), unsubstituted or substituted with alkyl, keto, fluoro, alkoxy, alkylthio, aryl, heteroaryl, cycloheteroalkyl, cycloheteroalkyl, cyano, aryloxy, cycloalkyl, COOR″, P═O(OR″)₂, CH₂P═O(OR″)₂, CF₂P═O(OR″)₂, NHCOCOOR″, CH(COOR″)₂;
aryl, unsubstituted or substituted with alkyl, keto, fluoro, alkoxy, alkylthio, cyano, aryloxy, COOR″, P═O(OR″)₂, CH₂P═O(OR″)₂, CF₂P═O(OR″)₂, NHCOCOOR″, CH(COOR″)₂;
heteroaryl, cycloheteroalkyl, cycloheteroalkyl, unsubstituted or substituted with alkyl, keto, fluoro, alkoxy, alkylthio, cyano, aryloxy, COOR″, P═O(OR″)₂, CH₂P═O(OR″)₂, CF₂P═O(OR″)₂, NHCOCOOR″, CH(COOR″)₂;
cycloalkyl, unsubstituted or substituted with alkyl, keto, fluoro, alkoxy, alkylthio, aryl, heteroaryl, cycloheteroalkyl, cycloheteroalkyl, cyano, COOR″, P═O(OR″)₂, CH₂P═O(OR″)₂, CF₂P—O(OR″)₂, NHCOCOOR″, CH(COOR″)₂;
Each R″ is independently
hydrogen,
alkyl (C1-C10), unsubstituted or substituted with alkyl, keto, fluoro, alkoxy, alkylthio, aryl, heteroaryl, cycloheteroalkyl, cycloheteroalkyl;
aryl, unsubstituted or substituted with alkyl, keto, fluoro, alkoxy, alkylthio;
heteroaryl, cycloheteroalkyl, unsubstituted or substituted with alkyl, keto, fluoro, alkoxy, alkylthio;
cycloalkyl, unsubstituted or substituted with alkyl, keto, fluoro, alkoxy, alkylthio, aryl, heteroaryl, cycloheteroalkyl;
Each R′″ is independently
alkyl (C1-C10), unsubstituted or substituted with alkyl, keto, fluoro, alkoxy, alkylthio, aryl, heteroaryl, cycloheteroalkyl, COOR″, P═O(OR″)₂, CH₂P═O(OR″)₂, CF₂P═O(OR″)₂, NHCOCOOR″, CH(COOR″)₂;
aryl, unsubstituted or substituted with alkyl, keto, fluoro, alkoxy, alkylthio;
heteroaryl, cycloheteroalkyl, unsubstituted or substituted with alkyl, keto, fluoro, alkoxy, alkylthio, COOR″, P═O(OR″)₂, CH₂P═O(OR″)₂, CF₂P═O(OR″)₂, NHCOCOOR″, CH(COOR″)₂;
cycloalkyl, unsubstituted or substituted with alkyl, keto, fluoro, alkoxy, alkylthio, aryl, heteroaryl, cycloheteroalkyl, COOR″, P═O(OR″)₂, CH₂P—O(OR″)₂, CF₂P═O(OR″)₂, NHCOCOOR″, CH(COOR″)₂; and
Each R″″ is independently
alkyl (C1-C10), unsubstituted or substituted with alkyl, keto, fluoro, alkoxy, alkylthio, aryl, heteroaryl, cycloheteroalkyl, COOR″, PO(OR″)₂, CH₂P═O(OR″)₂, CF₂P═O(OR″)₂, NHCOCOOR″, CH(COOR″)₂;
aryl, unsubstituted or substituted with alkyl, keto, fluoro, alkoxy, alkylthio, COOR″, P═O(OR″)₂, CH₂P═O(OR″)₂, CF₂P═O(OR″)₂, NHCOCOOR″, CH(COOR″)₂;
heteroaryl, cycloheteroalkyl, unsubstituted or substituted with alkyl, keto, fluoro, alkoxy, alkylthio, COOR″, P═O(OR″)₂, CH₂P═O(OR″)₂, CF₂P═O(OR″)₂, NHCOCOOR″, CH(COOR″)₂; and
cycloalkyl, unsubstituted or substituted with alkyl, keto, fluoro, alkoxy, alkylthio, aryl, heteroaryl, cycloheteroalky, COOR″, P═O(OR″)₂, CH₂P═O(OR″)₂, CF₂P═O(OR″)₂, NHCOCOOR″, CH(COOR″)₂;
and wherein each of R1, R2 and R3 are linked to their respective core atoms through C, N, O or S of the substiuent group, provided that if R2 is to be linked through O or S, then the core atom S is oxidized.

2. A method as recited in claim 1 wherein R1 and R2 are taken together with the core unit to which they are attached (formula I) to form a heterocyclic group having formula (II) as follows: 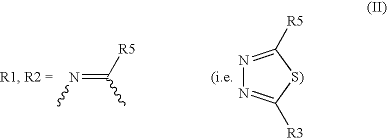
wherein R5 is an amino group with two substituents, where
one substituent is arylcarbonyl, arylmethylcarbonyl, arylsulfonyl, aryldimethyloxycarbonyl, or aryloxymethylcarbonyl, [where the aryl group is phenyl, benzox[c]1,2,5-oxadiazol-5-yl, 1-furyl, 2-furyl 1-naphthyl or 2-naphthyl, unsubstituted or substituted with one or more of the following or their combinations: perfluoroalkyl (C1-C4), alkyl (C1-C4), nitro, alkoxycarbonyl (C1-C4), carboxyl, carboxyalkyl(C1-C4), CF₂P═O(OH)₂, NHCOCOOH, phenoxy (unsubstituted or substituted with alkoxy (C1-C4), CF₂P═O(OH)₂, NHCOCOOH, COOH, and/or halogen), or phenylalkoxy (C1-C4)], hydrogen, CF₂P═O(OH)₂, NHCOCOOH, or a phenyl group [unsubstituted or with one or more of the following substituents or combinations: hydroxy, halogen, nitro, CF₂P═O(OH)₂, NHCOCOOH, carboxy, carboxyalkyl(C1-C4), carboxyalkylthio (C1-C6), phenyl, alkyl (C1-C10) or alkoxy (C1-C10) (unsubstituted or substituted with NR1R2, COOH, cycloheteroalkyl), perfluoroalkyl (C1-C4), alkoxycarbonyl (C1-C4), alkylthio (C1-C4), phenylalkoxy (C1-C4), phenylsulfonylamino (each unsubstituted or substituted on phenyl with alkyl (C1-C4)), phenoxy (unsubstituted or substituted on phenyl with nitro, perfluoroalkyl (C1-C4), carboxymethyl, carboxy, CF₂P═O(OH)₂, NHCOCOOH, alkoxycarbonylmethyl (C1-C4)), carboxyalkyl(C1-C4), phenylalkylthio (C1-C4, unsubstituted or substituted on phenyl with alkoxy (C1-C4), and/or phenyl), aminosulfonyl, alkylaminosulfonyl (C1-C4), dialkylaminosulfonyl (C1-C4 where the two alkyls unsubstituted or form a heteroalicyclic ring)]; and
the second substituent on the amino group forming R5 is hydrogen, alkyl (C1-C10) or alkoxy (C1-C10) (each unsubstituted or substituted with NR1R2, COOH, CF₂P═O(OH)₂, NHCOCOOH, cycloheteroalkyl), naphthylalkyl (C1-C4), phenylalkyl (C1-C4, with the phenyl group unsubstituted or substituted with phenyl, alkyl (C1-C4), halo, amino, amido, keto, CF₂P═O(OH)₂, NHCOCOOH, alkyl (C1-C10) or alkoxy (C1-C10) (unsubstituted or substituted with NR1R2, COOH, cycloheteroalkyl), nitro, carboxy, perfluoroalkylthio (C1-C4), halogen, CF₂P═O(OH)₂, NHCOCOOH, 1,2,3-thiadiazolyl, and/or alkoxy carbonyl (C1-C4)), alkyl (C1-C10), cycloalkyl (C4-C8, unsubstituted or substituted with alkyl (C1-C4)), or indanyl (unsubstituted or substituted with alkyl (C1-C4)).

3. A method as recited in claim 2 wherein R3 is
(1) a phenyl group unsubstituted or substituted with one to three of the following and their combinations: halogen, hydroxy, aryloxy, nitro, carboxylic acid, CF₂P═O(OH)₂, NHCOCOOH, alkyl (C1-C10) or alkoxy (C1-C10) (unsubstituted or substituted with NR1R2, COOH, cycloheteroalkyl), alkylthio (C1-C4), 2′-hydroxyethoxy, alkoxycarbonylmethoxy (C1-C4), dialkylamino (C1-C4 where the two alkyls can form a heteroalicyclic ring), 2-(dialkylamino)-2-oxoethoxy (C1-C7 where the two alkyls can form a heteroalicyclic ring), difluoromethoxy, perfluoroallyl (C1-C4), perfluoroalkylthio (C1-C4), perfluoroalkoxy (C1-C4), 2-carboxyvinyl, alkanoyl (C1-C5), alkoxycarbonyl (C1-C4), alkanoylamino (C1-C8), benzoylamino (unsubstituted or substituted with one or more perfluoroalkyl group (C1-C4) and/or CF₂P═O(OH)₂, NHCOCOOH,), aryl, aryloxy, arylcarbonyl, arylmethoxy, arylmethyl in which the methyl group is substituted with hydroxyl, O(CH₂)ₙCOOH (n=1-5), S(CH₂)ₙCOOH (n=1-5), (4-carboxy)benzyloxy, (3-carboxybenzyloxy), or the group ═N—O—CH₂R in which R is carboxyl, alkoxycarbonyl (C1-C4), hydrogen, or phenyl (unsubstituted or substituted with one or more halogens), or the group ═N—NHAr in which Ar is a phenyl (unsubstituted or substituted with one or more alkyl groups (C1-C4), and/or a carboxyl group, and/or CF₂P═O(OH)₂, NHCOCOOH), or the group-Y—(CH₂)ₙ-Z, where Y is O or S, n is 1, 2, or 3, and Z is hydrogen, methyl, branched alkyl (C3-C5), cycloalkyl (C3-C6), phenyl (unsubstituted or substituted with one or more of the following: halogen, trifluoroalkyl, carboxy, alkoxycarbonyl (C1-C4), CF₂P═O(OH)₂, NHCOCOOH, or carboxyl; or
(2) a pyridylthio group unsubstituted or substituted with one or more halogen and/or one or more nitro groups, methylenedioxyphenyl, benzo[3,4-c]1,2,5-oxadiazol-5-yl, 4-oxo-3-hydroquinazolin-2-yl, or a group having formula (III) as follows: 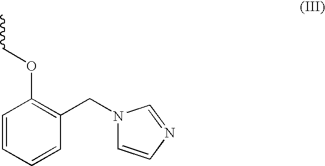
in which the imidazole ring is unsubstituted or substituted with one or more halogens.

4. A method as recited in claim 1 wherein R1 and R2 are taken together with the core unit to which they are attached (formula I) to form a heterocyclic group having formula (IV as follows: 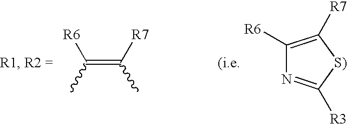
wherein R6 and R7 are each independently as defined for R1, R2 and R3.

5. A method as recited in claim 1 wherein R1 and R2 are linked through an aromatic ring, and taken together with the N═CR3—S unit to which they are attached, form a tricyclic heterocyclic group having formula (V) as follows: 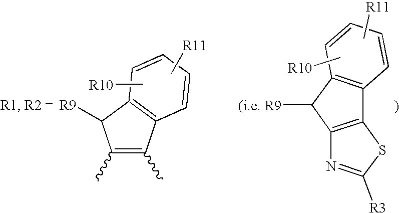
Where R9, R10 and R11 are each independently as defined for R1, R2 and R3.

6. A method as recited in claim 1 wherein R1 and R2, taken together with the N═CR3—S unit to which they are attached, form a heterocyclic group having formula (VI) as follows: 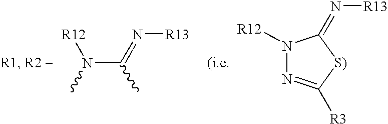
Wherein R12 and R13 are each independently as defined for R1, R2 and R3.

7. A method as recited in claim 1 wherein R1 and R3, taken together with the N═C—SR2 unit to which they are attached, form a heterocyclic group having formula (VII) as follows: 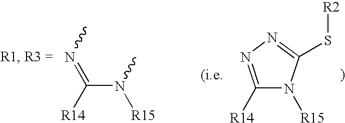
Wherein R14 and R15 are each independently as defined for R1, R2 and R3.

8. A method as recited in claim 1 wherein R1 and R3, taken together with the N═C—SR2 unit to which they are attached, form a bicylic heterocyclic group having formula (VII) as follows: 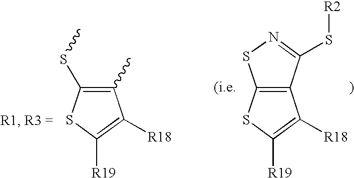
Wherein R18 and R19 are each independently as defined for R1, R2 and R3.

9. A method as recited in claim 1 wherein R1, R2 and R3, taken together with the N═C—S unit to which they are attached, form a bicyclic heterocyclic group having formula (IX) as follows: 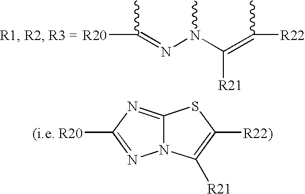
Wherein R20, R21 and R22 are each independently as defined for R1, R2 and R3.

10. A method as recited in claim 1 wherein R1, R2 and R3, taken together with the N═C—S unit to which they are attached, form a bicyclic heterocyclic group having formula (X) as follows: 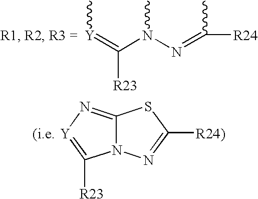
Where R23 and R24 are each independently as defined for R1, R2 and R3.

11. A pharmaceutical composition which comprises an effective amount of a compound having the formula: 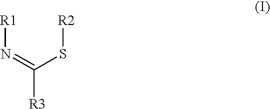
or a pharmaceutically acceptable salt thereof, wherein:
R1, R2, and R3 are each independently selected from
H, hydroxyl, alkoxy, alkylthio, nitro, amino or amido (each unsubstituted or substituted with alkyl, amino, cycloheteroalkyl, cycloalkyl fluoro, aryl, heteroaryl, cycloheteroalkyl, alkylthio, arylthio, cyano, OR′, OC═OR″, C═O—OR′″, or C═O—NR″″R″″);
small alkyl (C1-C10) (unsubstituted or substituted with alkyl, amino, cycloheteroalkyl, cycloalkyl fluoro, aryl, heteroaryl, cycloheteroalkyl, alkylthio, P═O(OR″)₂, CH₂P═O(OR″)₂, CF₂P═O(OR″)₂, NHCOCOOR″, CH(COOR″)₂, arylthio, cyano, OR″, OC═OR″, C═O—OR′″, or C═O—NR″″R″″);
phenyl and mono and disubstituted (at positions 3 and 4) phenyl (wherein the phenyl ring is independently substituted with alkyl, trifluoromethyl, mono and di halogen atoms, alkylthio, alkoxy, nitro, cyano, morphilino, cyclohexyl, phenyl, phenolic, dioxymethylene, nitro, acetylamino, OR′, P═O(OR″)₂, CH₂P═O(OR″)₂, CF₂P═O(OR″)₂, NHCOCOOR″, CH(COOR″)₂);
heteroaryl, cycloheteroalkyl and cycloheteroallyl (each unsubstituted or substituted with alkyl, halogen, alkylthio, alkoxy, or nitro, OR′, P═O(OR″)₂, CH₂P═O(OR″)₂, CF₂P═O(OR″)₂, NHCOCOOR″, CH(COOR″)₂,);
cycloalkyl (C3-C10) (unsubstituted or substituted with alkyl, fluoro, aryl, heteroaryl, cycloheteroalkyl, cycloheteroalkyl alkylthio, arylthio, cyano, P—O(OR″)₂, CH₂P═O(OR″)₂, CF₂P═O(OR″)₂, NHCOCOOR″, CH(COOR″)₂, OR′, OC═OR′, C═O—OR″, or C═O—NR′″R″″);
alkenyl (C1-C10) (unsubstituted or substituted with alkyl, fluoro, aryl, heteroaryl, cycloheteroalkyl, cycloheteroalkyl, alkylthio, arylthio, cyano, P═O(OR″)₂, CH₂P═O(OR″)₂, CF₂P═O(OR″)₂, NHCOCOOR″, CH(COOR″)₂, OR′, OC═OR′, C═O—OR″, or C═O—NR′″R″″);
alkadienyl (C1-C10) (unsubstituted or substituted with alkyl, fluoro, aryl, heteroaryl, cycloheteroalkyl, cycloheteroalkyl, alkylthio, arylthio, cyano, P═O(OR″)₂, CH₂P═O(OR″)₂, CF₂P═O(OR″)₂, NHCOCOOR″, CH(COOR″)₂, OR′, —OC═OR5, —C═O—OR″, C═O—NR′″R″″);
cycloalkenyl (C4-C10), unsubstituted or substituted with alkyl, fluoro, aryl, heteroaryl, cycloheteroalkyl, cycloheteroalkyl, alkylthio, arylthio, cyano, P═O(OR″)₂, CH₂P═O(OR″)₂, CF₂P═O(OR″)₂, NHCOCOOR″, CH(COOR″)₂, OR′, —OC═OR′, —C═O—OR″, C═O—NR′″R″″;
bicycloalkyl (C5-C12), unsubstituted or substituted with alkyl, fluoro, aryl, heteroaryl, cycloheteroalkyl, cycloheteroalkyl, alkylthio, arylthio, cyano, P═O(OR″)₂, CH₂P═O(OR″)₂, CF₂P═O(OR″)₂, NHCOCOOR″, CH(COOR″)₂, OR′, —OC═OR′, —C═O—OR″, C═O—NR′″R″″;
tricycloalkyl (C8-C14), unsubstituted or substituted with alkyl, fluoro, aryl, heteroaryl, cycloheteroalkyl, cycloheteroalkyl, alkylthio, arylthio, cyano, P═O(OR″)₂, CH₂P═O(OR″)₂, CF₂P═O(OR″)₂, NHCOCOOR″, CH(COOR″)₂, OR′, —OC═OR′, —C═O—OR″, C═O—NR′″R″″;
where
Each R′ is independently:
hydrogen;
alkyl (C1-C10), unsubstituted or substituted with alkyl, keto, fluoro, alkoxy, alkylthio, aryl, heteroaryl, cycloheteroalkyl, cycloheteroalkyl, cyano, aryloxy, cycloalkyl, COOR″, P═O(OR″)₂, CH₂P═O(OR″)₂, CF₂P═O(OR″)₂, NHCOCOOR″, CH(COOR″)₂;
aryl, unsubstituted or substituted with alkyl, keto, fluoro, alkoxy, alkylthio, cyano, aryloxy, COOR″, P═O(OR″)₂, CH₂P═O(OR″)₂, CF₂P═O(OR″)₂, NHCOCOOR″, CH(COOR″)₂;
heteroaryl, cycloheteroalkyl, cycloheteroalkyl, unsubstituted or substituted with alkyl, keto, fluoro, alkoxy, alkylthio, cyano, aryloxy, COOR″, P═O(OR″)₂, CH₂P═O(OR″)₂, CF₂P═O(OR″)₂, NHCOCOOR″, CH(COOR″)₂;
cycloalkyl, unsubstituted or substituted with alkyl, keto, fluoro, alkoxy, alkylthio, aryl, heteroaryl, cycloheteroalkyl, cycloheteroalkyl, cyano, COOR″, P═O(OR″)₂, CH₂P═O(OR″)₂, CF₂P═O(OR″)₂, NHCOCOOR″, CH(COOR″)₂;
Each R″ is independently
hydrogen,
alkyl (C1-C10), unsubstituted or substituted with alkyl, keto, fluoro, alkoxy, alkylthio, aryl, heteroaryl, cycloheteroalkyl, cycloheteroalkyl;
aryl, unsubstituted or substituted with alkyl, keto, fluoro, alkoxy, alkylthio;
heteroaryl, cycloheteroalkyl, unsubstituted or substituted with alkyl, keto, fluoro, alkoxy, alkylthio;
cycloalkyl, unsubstituted or substituted with alkyl, keto, fluoro, alkoxy, alkylthio, aryl, heteroaryl, cycloheteroalkyl;
Each R′″ is independently
alkyl (C1-C10), unsubstituted or substituted with alkyl, keto, fluoro, alkoxy, alkylthio, aryl, heteroaryl, cycloheteroalkyl, COOR″, P═O(OR″)₂, CH₂P═O(OR″)₂, CF₂P═O(OR″)₂, NHCOCOOR″, CH(COOR″)₂;
aryl, unsubstituted or substituted with alkyl, keto, fluoro, alkoxy, alkylthio;
heteroaryl, cycloheteroalkyl, unsubstituted or substituted with alkyl, keto, fluoro, alkoxy, alkylthio, COOR″, P═O(OR″)₂, CH₂P═O(OR″)₂, CF₂P═O(OR″)₂, NHCOCOOR″, CH(COOR″)₂;
cycloalkyl, unsubstituted or substituted with alkyl, keto, fluoro, alkoxy, alkylthio, aryl, heteroaryl, cycloheteroalkyl, COOR″, P═O(OR″)₂, CH₂P═O(OR″)₂, CF₂P═O(OR″)₂, NHCOCOOR″, CH(COOR″)₂; and
Each R″″ is independently
alkyl (C1-C10), unsubstituted or substituted with alkyl, keto, fluoro, alkoxy, alkylthio, aryl, heteroaryl, cycloheteroalkyl, COOR″, P═O(OR″)₂, CH₂P═O(OR″)₂, CF₂P═O(OR″)₂, NHCOCOOR″, CH(COOR″)₂;
aryl, unsubstituted or substituted with alkyl, keto, fluoro, alkoxy, alkylthio, COOR″, P═O(OR″)₂, CH₂P═O(OR″)₂, CF₂P═O(OR″)₂, NHCOCOOR″, CH(COOR″)₂;
heteroaryl, cycloheteroalkyl, unsubstituted or substituted with alkyl, keto, fluoro, alkoxy, alkylthio, COOR″, P═O(OR″)₂, CH₂P═O(OR″)₂, CF₂P═O(OR″)₂, NHCOCOOR″, CH(COOR″)₂; and
cycloalkyl, unsubstituted or substituted with alkyl, keto, fluoro, alkoxy, alkylthio, aryl, heteroaryl, cycloheteroalky, COOR″, P═O(OR″)₂, CH₂P═O(OR″)₂, CF₂P═O(OR″)₂, NHCOCOOR″, CH(COOR″)₂;
and wherein each of R1, R2 and R3 are linked to their respective core atoms through C, N, O or S of the substiuent group, provided that if R2 is to be linked through O or S, then the core atom S is oxidized.

12. A pharmaceutical composition as recited in claim 11 wherein R1 and R2 are taken together with the core unit to which they are attached (formula I) to form a heterocyclic group having formula (II) as follows: 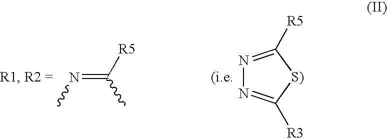
wherein R5 is an amino group with two substituents, where
one substituent is arylcarbonyl, arylmethylcarbonyl, arylsulfonyl, aryldimethyloxycarbonyl, or aryloxymethylcarbonyl, [where the aryl group is phenyl, benzox[c]1,2,5-oxadiazol-5-yl, 1-furyl, 2-furyl 1-naphthyl or 2-naphthyl, unsubstituted or substituted with one or more of the following or their combinations: perfluoroalkyl (C1-C4), alkyl (C1-C4), nitro, alkoxycarbonyl (C1-C4), carboxyl, carboxyalkyl(C1-C4), CF₂P═O(OH)₂, NHCOCOOH, phenoxy (unsubstituted or substituted with alkoxy (C1-C4), CF₂P═O(OH)₂, NHCOCOOH, COOH, and/or halogen), or phenylalkoxy (C1-C4)], hydrogen, CF₂P═O(OH)₂, NHCOCOOH, or a phenyl group [unsubstituted or with one or more of the following substituents or combinations: hydroxy, halogen, nitro, CF₂P═O(OH)₂, NHCOCOOH, carboxy, carboxyalkyl(C1-C4), carboxyalkylthio (C1-C6), phenyl, alkyl (C1-C10) or alkoxy (C1-C10) (unsubstituted or substituted with NR1R2, COOH, cycloheteroalkyl), perfluoroalkyl (C1-C4), alkoxycarbonyl (C1-C4), alkylthio (C1-C4), phenylalkoxy (C1-C4), phenylsulfonylamino (each unsubstituted or substituted on phenyl with alkyl (C1-C4)), phenoxy (unsubstituted or substituted on phenyl with nitro, perfluoroalkyl (C1-C4), carboxymethyl, carboxy, CF₂P═O(OH)₂, NHCOCOOH, alkoxycarbonylmethyl (C1-C4)), carboxyalkyl(C1-C4), phenylalkylthio (C1-C4, unsubstituted or substituted on phenyl with alkoxy (C1-C4), and/or phenyl), aminosulfonyl, alkylaminosulfonyl (C1-C4), dialkylaminosulfonyl (C1-C4 where the two alkyls unsubstituted or form a heteroalicyclic ring)]; and
the second substituent on the amino group forming R5 is hydrogen, alkyl (C1-C10) or alkoxy (C1-C10) (each unsubstituted or substituted with NR1R2, COOH, CF₂P═O(OH)₂, NHCOCOOH, cycloheteroalkyl), naphthylalkyl (C1-C4), phenylalkyl (C1-C4, with the phenyl group unsubstituted or substituted with phenyl, alkyl (C1-C4), halo, amino, amido, keto, CF₂P═O(OH)₂, NHCOCOOH, alkyl (C1-C10) or alkoxy (C1-C10) (unsubstituted or substituted with NR1R2, COOH, cycloheteroalkyl), nitro, carboxy, perfluoroalkylthio (C1-C4), halogen, CF₂P═O(OH)₂, NHCOCOOH, 1,2,3-thiadiazolyl, and/or alkoxy carbonyl (C1-C4)), alkyl (C1-C10), cycloalkyl (C4-C8, unsubstituted or substituted with alkyl (C1-C4)), or indanyl (unsubstituted or substituted with alkyl (C1-C4)).

13. A pharmaceutical composition as recited in claim 12 wherein R3 is
(1) a phenyl group unsubstituted or substituted with one to three of the following and their combinations: halogen, hydroxy, aryloxy, nitro, carboxylic acid, CF₂P═O(OH)₂, —NHCOCOOH, alkyl (C1-C10) or alkoxy (C1-C10) (unsubstituted or substituted with NR1R2, COOH, cycloheteroalkyl), alkylthio (C1-C4), 2′-hydroxyethoxy, alkoxycarbonylmethoxy (C1-C4), dialkylamino (C1-C4 where the two alkyls can form a heteroalicyclic ring), 2-(dialkylamino)-2-oxoethoxy (C1-C7 where the two alkyls can form a heteroalicyclic ring), difluoromethoxy, perfluoroalkyl (C1-C4), perfluoroalkylthio (C1-C4), perfluoroalkoxy (C1-C4), 2-carboxyvinyl, alkanoyl (C1-C5), alkoxycarbonyl (C1-C4), alkanoylamino (C1-C8), benzoylamino (unsubstituted or substituted with one or more perfluoroalkyl group (C1-C4) and/or CF₂P═O(OH)₂, NHCOCOOH,), aryl, aryloxy, arylcarbonyl, arylmethoxy, arylmethyl in which the methyl group is substituted with hydroxyl, O(CH₂)ₙCOOH (n=1-5), S(CH₂)ₙCOOH (n=1-5), (4-carboxy)benzyloxy, (3-carboxybenzyloxy), or the group ═N—O—CH₂R in which R is carboxyl, alkoxycarbonyl (C1-C4), hydrogen, or phenyl (unsubstituted or substituted with one or more halogens), or the group ═N—NHAr in which Ar is a phenyl (unsubstituted or substituted with one or more alkyl groups (C1-C4), and/or a carboxyl group, and/or CF₂P═O(OH)₂, NHCOCOOH), or the group-Y—(CH₂)ₙ-Z, where Y is O or S, n is 1, 2, or 3, and Z is hydrogen, methyl, branched alkyl (C3-C5), cycloalkyl (C3-C6), phenyl (unsubstituted or substituted with one or more of the following: halogen, trifluoroalkyl, carboxy, alkoxycarbonyl (C1-C4), CF₂P═O(OH)₂, NHCOCOOH, or carboxyl; or
(2) a pyridylthio group either unsubstituted or substituted with one or more halogen and/or one or more nitro groups, methylenedioxyphenyl, benzo[3,4-c]1,2,5-oxadiazol-5-yl, 4-oxo-3-hydroquinazolin-2-yl, or a group having formula (III) as follows: 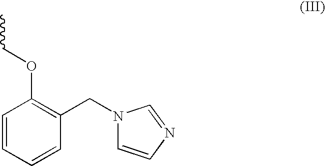
in which the imidazole ring is unsubstituted or substituted with one or more halogens.

14. A pharmaceutical composition as recited in claim 11 wherein R1 and R2 are taken together with the core unit to which they are attached (formula I) to form a heterocyclic group having formula (IV) as follows: 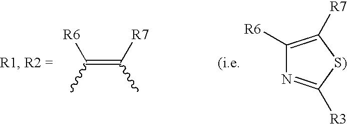
wherein R6 and R7 are each independently as defined for R1, R2 and R3.

15. A pharmaceutical composition as recited in claim 11 wherein R1 and R2 are linked through an aromatic ring, and taken together with the N═CR3—S unit to which they are attached, form a tricyclic heterocyclic group having formula (V) as follows: 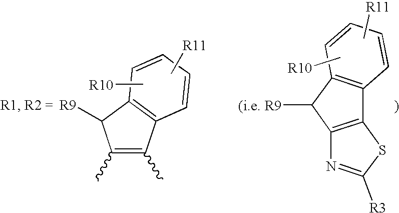
Where R9, R10 and R11 are each independently as defined for R1, R2 and R3.

16. A pharmaceutical composition as recited in claim 11 wherein R1 and R2, taken together with the N═CR3—S unit to which they are attached, form a heterocyclic group having formula (VI) as follows: 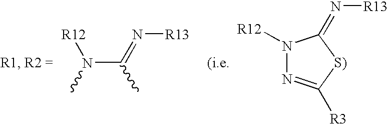
Wherein R12 and R13 are each independently as defined for R1, R2 and R3.

17. A pharmaceutical composition as recited in claim 11 wherein R1 and R3, taken together with the N═C—SR2 unit to which they are attached, form a heterocyclic group having formula (VII) as follows: 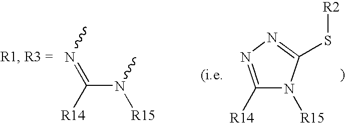
Wherein R14 and R15 are each independently as defined for R1, R2 and R3.

18. A pharmaceutical composition as recited in claim 11 wherein R1 and R3, taken together with the N═C—SR2 unit to which they are attached, form a bicylic heterocyclic group having formula (VIII) as follows: 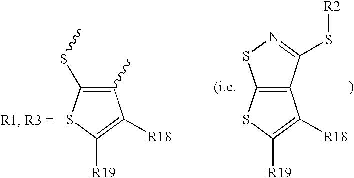
Wherein R18 and R19 are each independently as defined for R1, R2 and R3.

19. A pharmaceutical composition as recited in claim 11 wherein R1, R2 and R3, taken together with the N═C—S unit to which they are attached, form a bicyclic heterocyclic group having formula (IX) as follows: 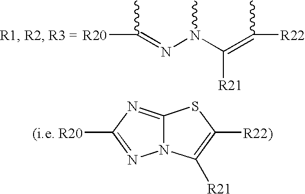
Wherein R20, R21 and R22 are each independently as defined for R1, R2 and R3.

20. A pharmaceutical composition as recited in claim 11 wherein R1, R2 and R3, taken together with the N═C—S unit to which they are attached, form a bicyclic heterocyclic group having formula (X) as follows: 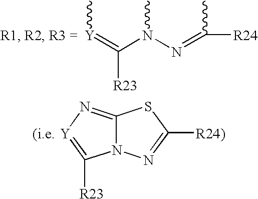
Where R23 and R24 are each independently as defined for R1, R2 and R3.
StructureNameTable

| Molecular Structure | Molecular Name |
|---|---|
| 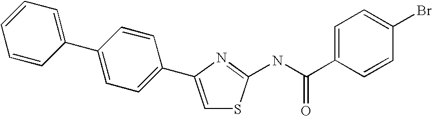 | N-(4-biphenyl-4-yl-thiazol-2-yl)-4-bromo-benz- amide |
| 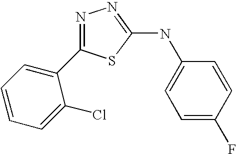 | N2-(4-fluorophenyl)-5-(2-chlorophenyl)-1,3,4- thiadiazol-2-amine |
| 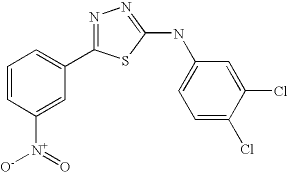 | N2-(3,4-dichlorophenyl)-5-(3-nitrophenyl)-1,3,4- thiadiazol-2-amine |
| 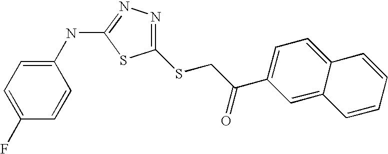 | 2-[[5-(4-fluoroanilino)-1,3,4-thiadiazol-2- yl]thio]-1-(2-naphthyl)ethan-1-one |
| 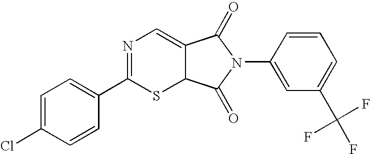 | 2-(4-chlorophenyl)-6-[3-(trifluoromethyl)phenyl]- 7aH-azolidino[3,4-e]1,3-thiazine-5,7-dione |
| 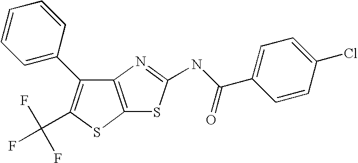 | 4-chloro-N-[6-phenyl-5-(trifluoromethyl)thieno[3,2- d][1,3]thiazol-2-yl]benzamide |
| 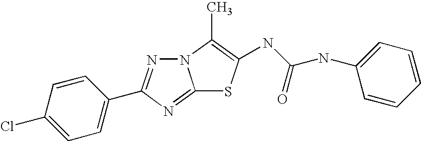 | N-[2-(4-chlorophenyl)-6-methyl(1,3-thiazolino[3,2- d]1,2,4-triazol-5-yl)](phenylamino)carboxamide |
| 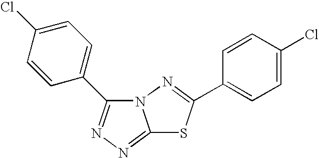 | 3,6-di(4-chlorophenyl)[1,2,4]triazol[3,4-b][1,3,4]thiadiazole |
| 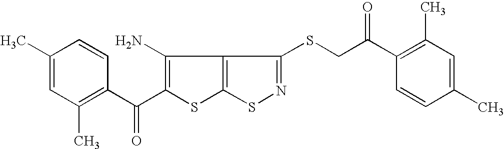 | 2-{4-amino-5-[(2,4-dimethylphenyl)carbonyl]thiopheno[3,2-d]isothiazol-3-ylthio}-1-(2,4-dimethylphenyl)ethan-1-one |
| 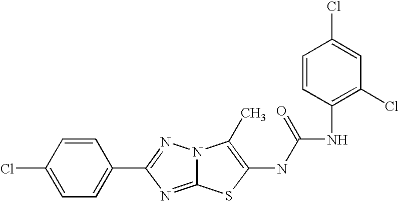 | [(2,4-dichlorophenyl)amino]-N-[2-(4-chloro- phenyl)-6-methyl(1,3-thiazolino[3,2-d]1,2,4-triazol-5-yl)]carboxamide |
| 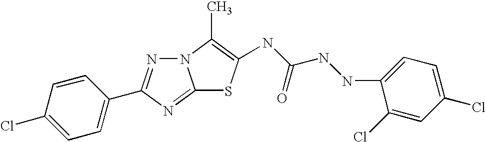 | [2-(2,4-dichlorophenyl)hydrazino]-N-[2-(4-chloro- phenyl)-6-methyl(1,3-thiazolino[3,2-d]1,2,4-triazol-5-yl)]carboxamide |
| 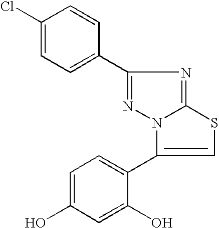 | 4-=[2-(4-chlorophenyl)[1,3]thiazolo[3,2-B][1,2,4]triazol-6-yl]benzene-1,3-diol |
| 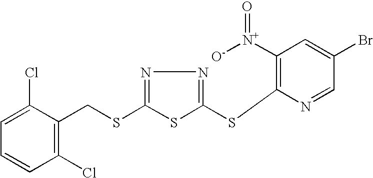 | 2-{5-[(2,6-dichlorophenyl)methylthio](1,3,4- thiadiazol-2-ylthio)}-5-bromo-3-nitropyridine |
| 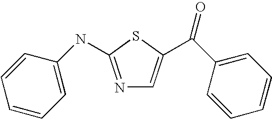 | (2-anilino-1,3-thiazol-5-yl)(phenyl)methanone |
| 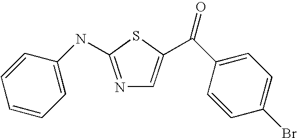 | (2-anilino-1,3-thiazol-5-yl)(4-bromo- phenyl)methanone |
| 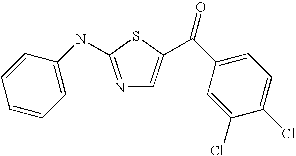 | (2-anilino-1,3-thiazol-5-yl)(3,4-dichloro- phenyl)methanone |
| 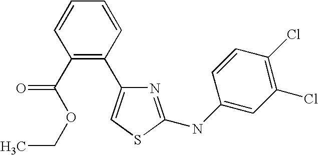 | ethyl 2-[2-(3,4-dichloroanilino)-1,3-thiazol- 4-yl]benzenecarboxylate |
| 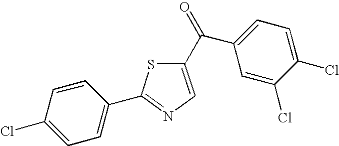 | [2-(4-chlorophenyl)-1,3-thiazol-5-yl](3,4- dichlorophenyl)methanone |
| 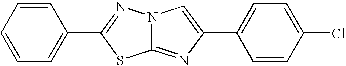 | 6-(4-chlorophenyl)-2-phenylimidazolo[2,1- b]1,3,4-thiadiazoline |
| 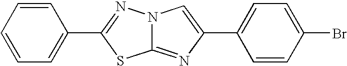 | 6-(4-bromophenyl)-2-phenylimidazolo[2,1- b]1,3,4-thiadiazoline |
| 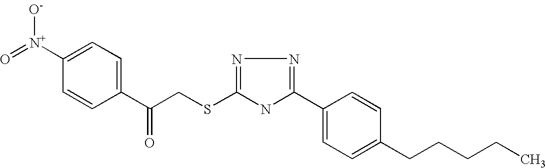 | 1-(4-nitrophenyl)-2-[5-(4-pentylphenyl)(4H- 1,2,4-triazol-3-ylthio)]ethan-1-one |
| 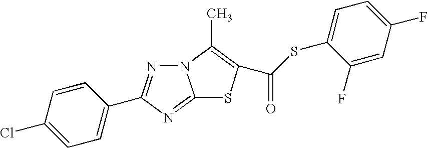 | 2,4-difluorophenylthio 2-(4-chlorophenyl)-6- methyl(1,3-thiazolino[3,2-d]1,2,4-triazol-5-yl) ketone |
| 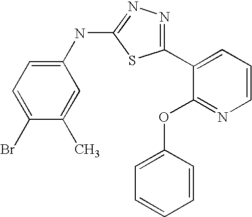 | N2-(4-bromo-3-methylphenyl)-5-(2-phenoxy-3- pyridyl)-1,3,4-thiadiazol-2-amine |
| 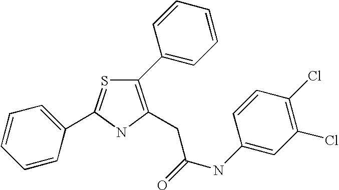 | N-(3,4-dichlorophenyl)-2-(2,4-diphenyl-1,3- thiazol-5-yl)acetamide |
| 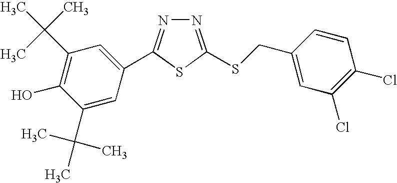 | 2,4-difluorophenylthio 2-(4-chlorophenyl)-6- methyl(1,3-thiazolino[3,2-d]1,2,4-5-yl) ketone |
| 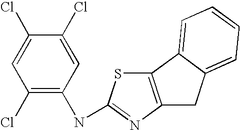 | N2-(2,4,5-trichlorophenyl)-8h-indeno[1,2-d][1,3]thiazol-2-amin hydrobromide |
| 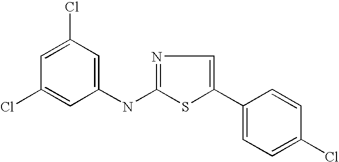 | (3,5-dichlorophenyl)[5-(4-chlorophenyl)(1,3- thiazol-2-yl)]amine |
| 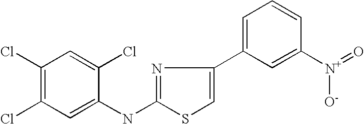 | N2-(2,4,5-trichlorophenyl)-4-(3-nitrophenyl)-1,3- thiazol-2-amine |
| 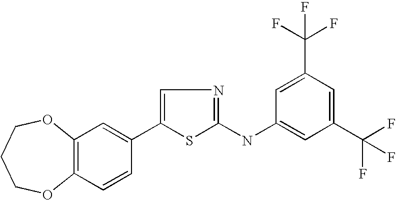 | (5-(2H,3H,4H-benzo[3,4-b]1,4-dioxepin-7-yl)(1,3- thiazol-2-yl))[3,5-bis(trifluoromethyl)phenyl]amine |
| 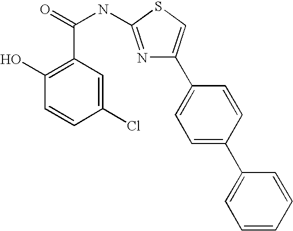 | N-(4-[1,1′-biphenyl]-4-yl-1,3-thiazol-2-yl)- 5-chloro-2-hydroxybenzamide |
| 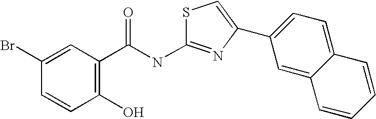 | N1-[4-(2-naphthyl)-1,3-thiazol-2-yl]-5-bromo-2- hydroxybenzamide |
| 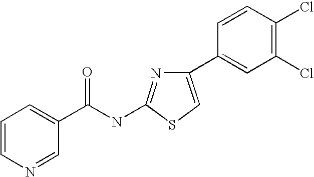 | N-[4-(3,4-dichlorophenyl)(1,3-thiazol-2-yl)]-3- pyridylcarboxamide |
| 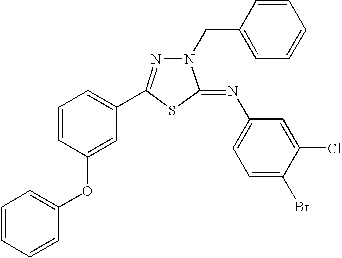 | 3-{2-[aza(4-bromo-3-chlorophenyl)methylene]-3- benzyl(1,3,4-thiadiazolin-5-yl)}-1-phenoxybenzene |
| 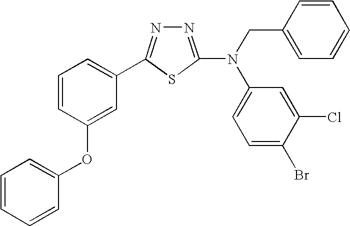 | (4-bromo-3-chlorophenyl)benzyl[5-(3-phenoxyphenyl) (1,3,4-thiadiazol-2-yl)]amine |
| 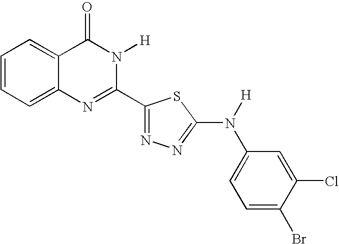 | 2-{5-[(4-benzo-3-chlorophenyl)amino]-1,3,4- thiadiazol-2-yl}-3-hydroquinazolin-4-one |
| 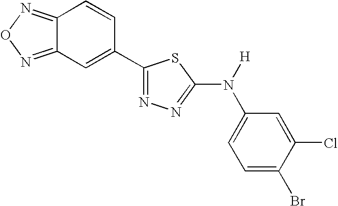 | (5-benzo[3,4-c]1,2,5-oxadiazol-5-yl(1,3,4- thiadiazol-2-yl))(4-bromo-3-chlorophenyl)amine |
| 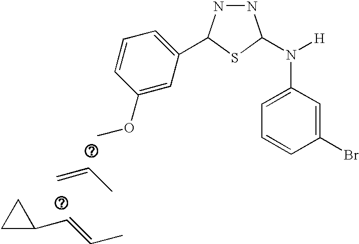 | (3-bromophenyl)[5-(3-{[3-(trifluoromethyl)- phenyl]methoxy}phenyl)(1,3,4-thiadiazol-2-yl)]amine |
| 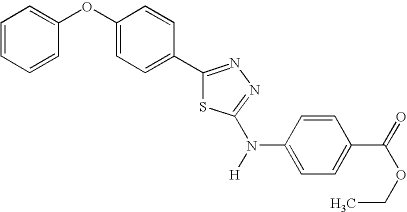 | ethyl 4-{[5-(4-phenoxyphenyl)-1,3,4-thiadiazol-2- yl]amino}benzoate |
| 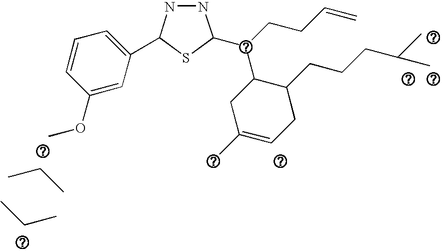 | (3,4-dichlorophenyl)[5-(3-{[4-(tert-butyl)phenyl]methoxy}phenyl)(1,3,4-thiadiazol-2-yl)]{[4-(tertbutyl)phenyl]methyl}amine |
| 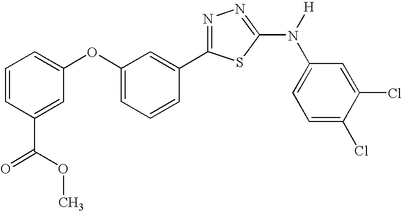 | methyl-3-(3-{5-[(3,4-dichlorophenyl)amino]-1,3,4- thiadiazol-2-yl}phenoxy)benzoate |
| 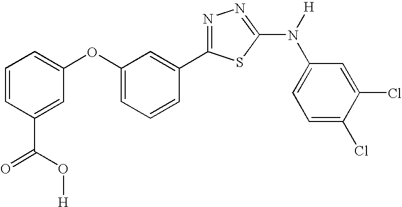 | 3-(3-{5-[(3,4-dichlorophenyl)amino]-1,3,4-thiadiazol- 2-yl}phenoxcy)benzoic acid |
| 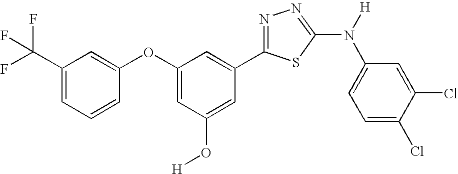 | 5-{5-[(3,4-dichlorophenyl)amino](1,3,4-thiadiazol- 2-yl)}-3-[3-(trifluoromethyl)phenoxy] phenol |
| 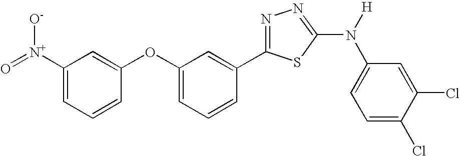 | (3,4-dichlorophenyl){5-[3-(3-nitrophenoxy)phenyl](1,3,4-thiadiazol-2-yl)}amine |
| 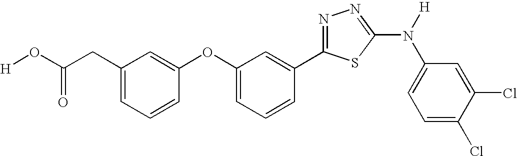 | 2-[3-(3-{5-[(3,4-dichlorophenyl)amino]-1,3,4- thiadiazol-2-yl}phenoxy)phenyl]acetic acid |
| 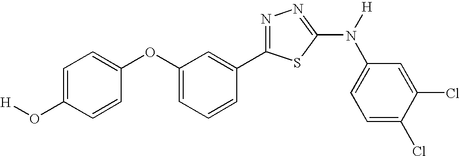 | 4-(3-{5-[(3,4-dichlorophenyl)amino]-1,3,4- thiadiazol-2-yl}phenoxy)phenol |
| 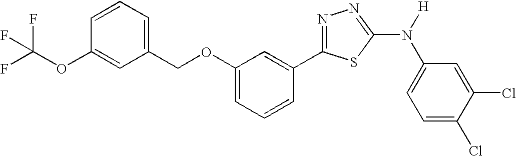 | (3,4-dichlorophenyl)[5-(3-{[3-(trifluoro- methoxy)phenyl]methoxy}phenyl)(1,3,4-thiadiazol-2-yl)]amine |
| 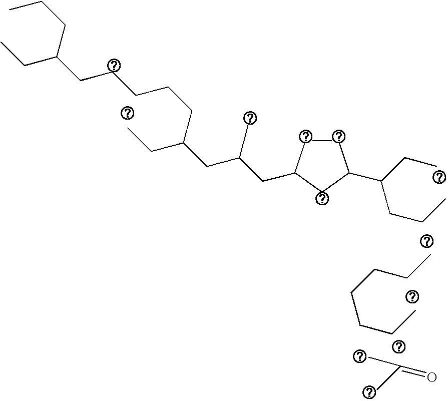 | 2-[4-(phenylmethoxy)phenyl]-N-(5-{3-[3-(tri- fluoromethyl)phenoxy]phenyl}(1,3,4-thiadiazol-2-yl))acetamide |
| 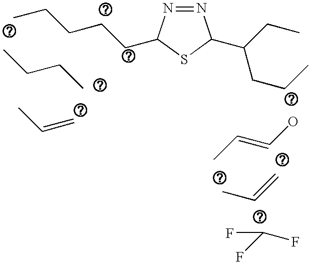 | naphthyl-N-(5-{3-[3-(trifluoromethyl)phenoxy]- phenyl}(1,3,4-thiadiazol-2-yl))carboxamide |
| 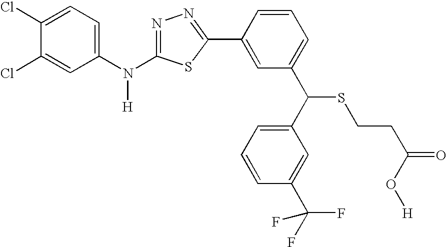 | 3-[(3-{5-[(3,4-dichlorophenyl)amino](1,3,4- thiadiazol-2-yl)}phenyl)[3-(trifluoromethyl)phenyl]methylthio]propanoic acid |
| 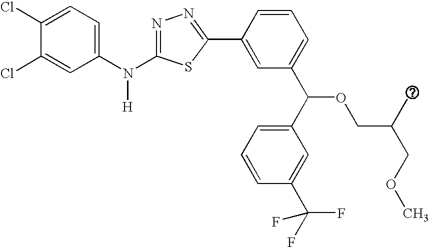 | ethyl 2-[(3-{5-[(3,4-dichlorophenyl)amino]- (1,3,4-thiadiazol-2-yl)}phenyl)[3-(trifluoromethyl)phenyl]meethoxy]acetate |
| 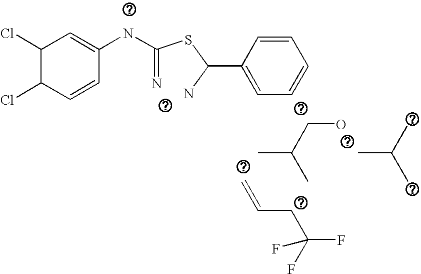 | 2-[(3-{5-[(3,4-dichlorophenyl)amino](1,3,4- thiadiazol-2-yl)}phenyl)[3-(trifluoromethyl)phenyl]methoxy]acetic acid |
| 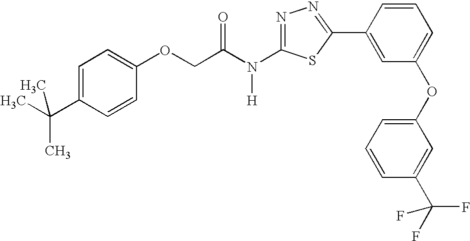 | 2-[4-(tert-butyl)phenoxy]-N-(5-{3-[3-(tri- fluoromethyl)phenoxy]phenyl}(1,3,4-thiadiazol-2-yl))acetamide |
| 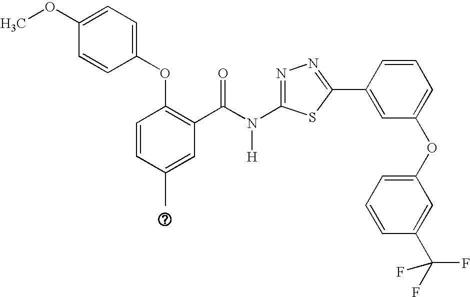 | [2-(4-methoxyphenoxy)-5-nitrophenyl]-N-(5-{3- [3-(trifluoromethyl)phenoxy]phenyl}(1,3,4-thiadiazol-2-yl))carboxamide |
| 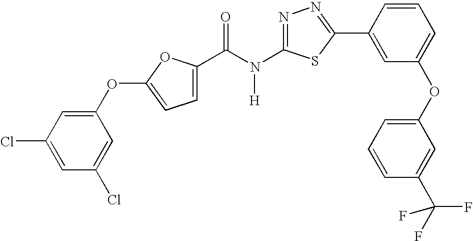 | [5-(3,5-dichlorophenoxy)(2-furyl)]-N-(5-{3-[3- (trifluoromethyl)phenoxy]phenyl}(1,3,4-thiadiazol-2-yl))carboxamide |
| 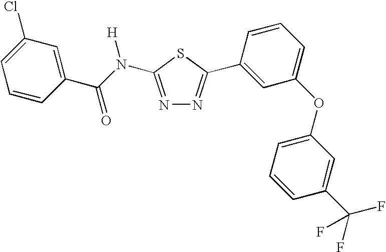 | (3-nitrophenyl)-N-(5-{3-[3-(trifluoromethyl)phen- oxy]phenyl}(1,3,4-thiadiazol-2-yl))carboxamide |
| 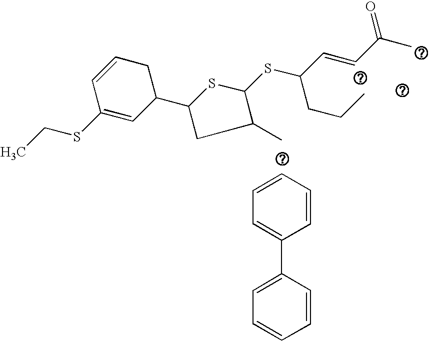 | meethyl 3-(azaq{5-(3-ethoxyphenyl)-3-[(4-phenyl- phenyl)methyl]*1,3,4-thiazolidin-2-ylidene)}methyl)benzoate |
| 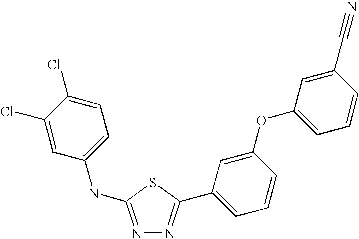 | 3-(3-{5-[(3,4-dichlorophenyl)amino]-1,3,4-thia- diazol-2-yl}phenoxy)benzenecarbonitrile |
| 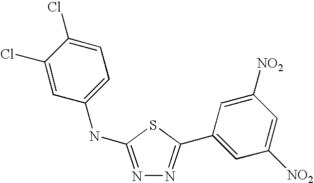 | 3-(3-{5-[(3,4-dichlorophenyl)amino]-1,3,4-thia- diazol-2-yl}phenoxy)benzenecarbonitrile |
| 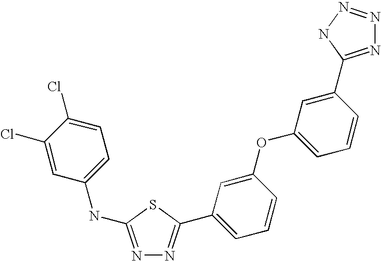 | {5-[3-(3-(1H-1,2,3,4-tetrazol-5-yl)phenoxy)phenyl]- (1,3,4-thiadiazol-2-yl)}(3,4-dichlorophenyl)amine |
| 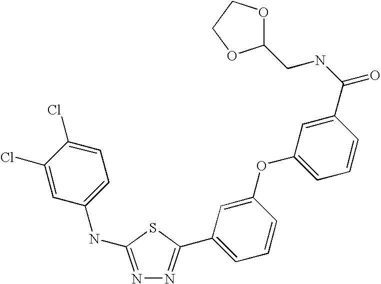 | [3-(3-{5-[(3,4-dichlorophenyl)amino](1,3,4-thia- diazol-2-yl)}phenoxy)phenyl]-N-(1,3-dioxolan-2-ylmethyl)carboxamide |
| 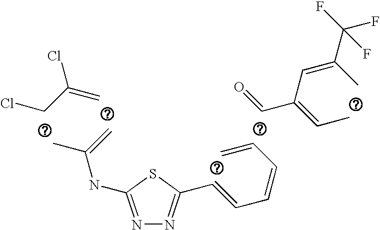 | 3-{5-[(3,4-dichlorophenyl)amino](1,3,4-thiadiazol- 2-yl)}phenyl 3-(trifluoromethyl)phenyl ketone |
| 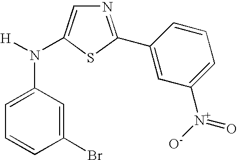 | (3-bromophenyl)[2-(3-nitrophenyl)(1,3-thiazol-5- yl)]amine |
| 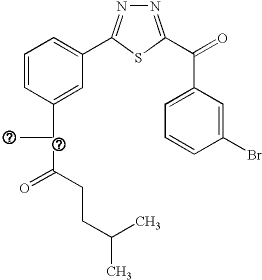 | N-(3-{5-[(3-bromophenyl)amino](1,3,4-thiadiazol- 2-yl)}phenyl)-4-methylpentanamide |
| 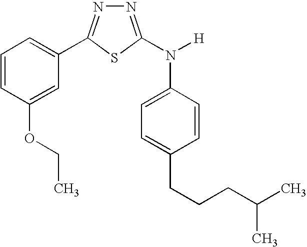 | [5-(3-ethoxyphenyl)(1,3,4-thiadiazol-2-yl)][4- (3-methylbutylthio)phenyl]amine |
| 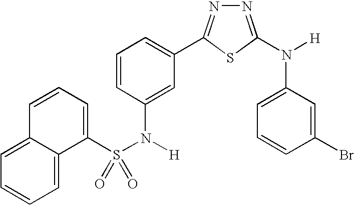 | (3-{5-[(3-bromophenyl)aqmino](1,3,4-thiadiazol- 2-yl)}phenyl)(naphthylsulfonyl)amine |
| 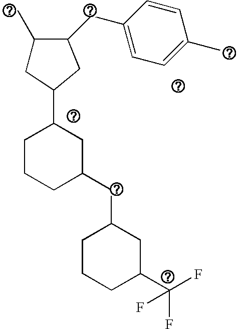 | 3-{2-[aza(3,4-dichlorophenyl)methylene]-3- methyl(1,3,4-thiadiazolin-5-yl)}-1-[3-(trifluoromethyl)phenoxy]benzene |
| 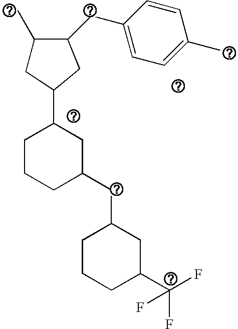 | (3,4-dichlorophenyl)methyl(5-{3-[3-(trifluoro- methyl)phenopxy]phenyl}(1,3,4-thiadiazol-2-yl))amine |
| 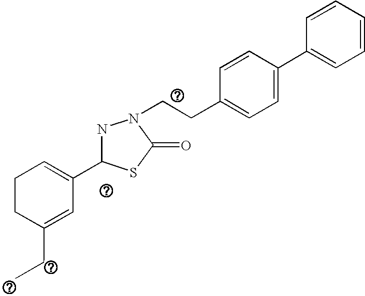 | 3-methoxy-1-[6-(4-phenylphenyl)imidazolo[2,1- b]1,3,4-thiadiazolin-2-yl]benzene |
| 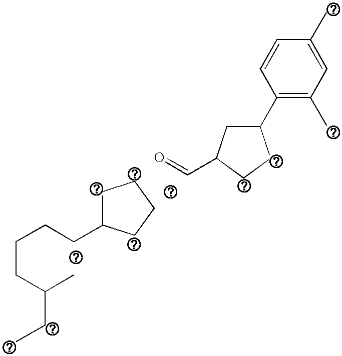 | 1-{6-[3-(2,4-dichlorophenyl)isoxazol-5-yl]- imidazolo[2,1-b]1,3,4-thiadiazoln-2-yl}-3-methoxybenzene |
| 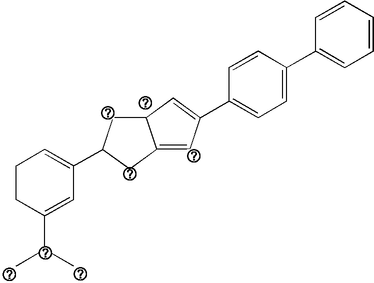 | 2-(3-nitrophenyl)-6-(4-phenylphenyl)imidazolo[2,1- b]1,3,4-thiadiazoline |
| 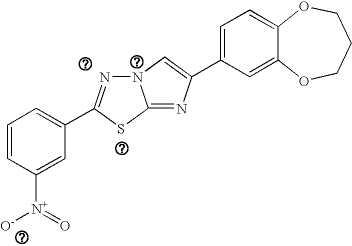 | 6-(2H,3H,4H-benzo[b]1,4-dioxepan-7-yl)-2-(3-nitro- phenyl)imidazolo[2,1-b]1,3,4-thiadiazoline |
| 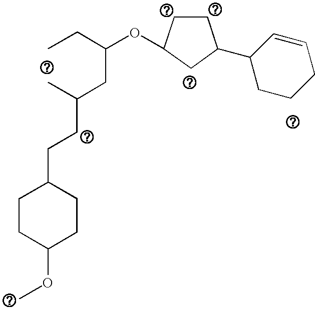 | {3-[(4-methoxyphenyl)methylthio]phenyl}[5- (3-nitrophenyl)(1,3,4-thiadiazol-2-yl)]amine |
| 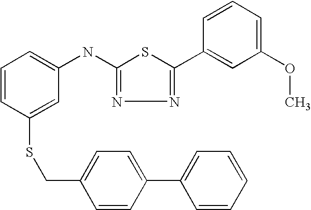 | [5-(3-methoxyphenyl)(1,3,4-thiadiazol-2-yl)]{3- [(4-phenylphenyl)methylthio]phenyl}amine |
| 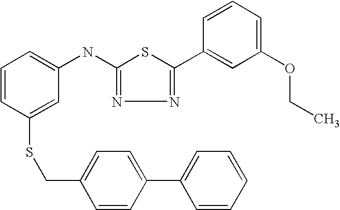 | [5-(3-ethoxyphenyl)(1,3,4-thiadiazol-2-yl)]{3- [(4-phenylphenyl)methylthio]phenyl}amine |
| 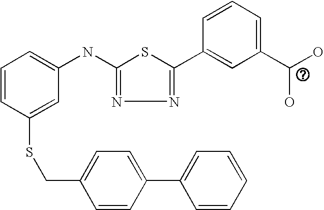 | [5-(3-nitrophenyl;)(1,3,4-thiadiazol-2-yl)]{3- [(4-phenylphenyl)methylthio]phenyl}amine |
| 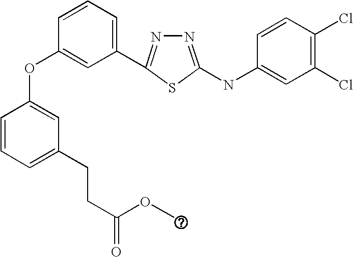 | 3-[3-(3-{5-[(3,4-dichlorophenyl)amino]-1,3,4- thiadiazol-2-yl}phenoxy)phenyl]propanoic acid |
| 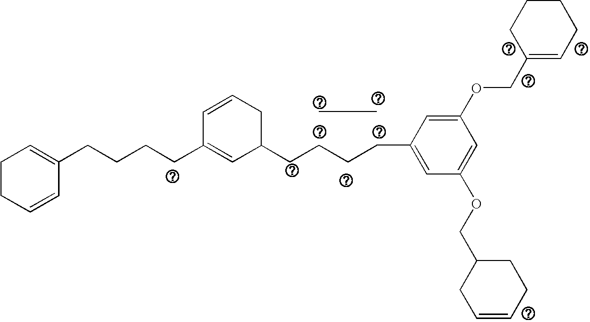 | {5-[3,5-bis(phenylmethoxy)phenyl](1,3,4-thia- diazol-2-yl)}[3-(3-phenylpropylthio)phenyl]amine |
| 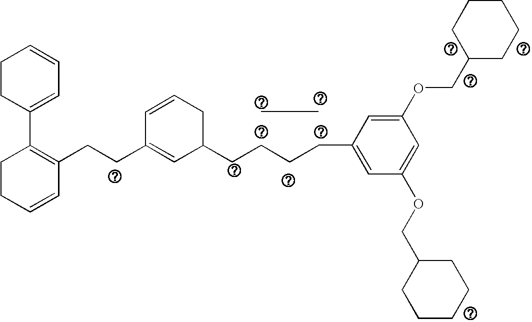 | {5-[3,5-bis(phenylmethoxy)phenyl](1,3,4-thia- diazol-2-yl)}{3-[(2-phenylphenyl)methylthio]phenyl}amine |
| 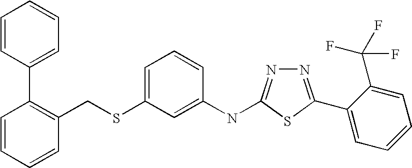 | {3-[(2-phenylphenyl)methylthio]phenyl}{5- [2-(trifluoromethyl)phenyl](1,3,4-thiadiazol-2-yl)}amine |
| 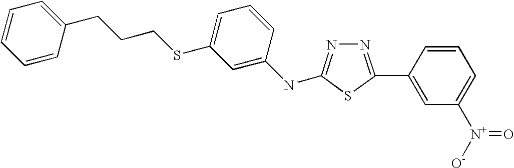 | [5-(3-nitrophenyl)(1,3,4-thiadiazol-2-yl)][3-(3- phenylpropylthio)phenyl]amine |
| 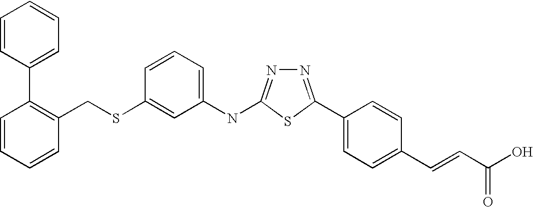 | 3-{4-[5-({3-[(2-phenylphenyl)methylthio]- phenyl}amino)(1,3,4-thiadiazol-2-yl)]phenyl}prop-2-enoic acid |
| 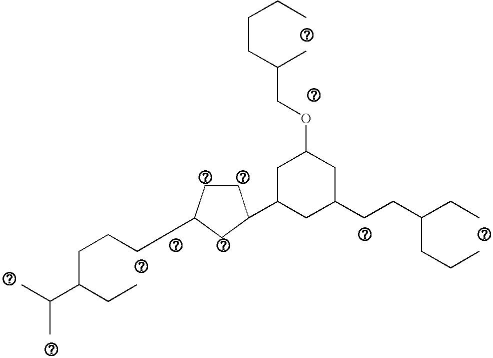 | 4-({5-[3,5-bis(phenylmethoxy)phenyl]-1,3,4-thia- diazol-2-yl}amino)benzoic acid |
| 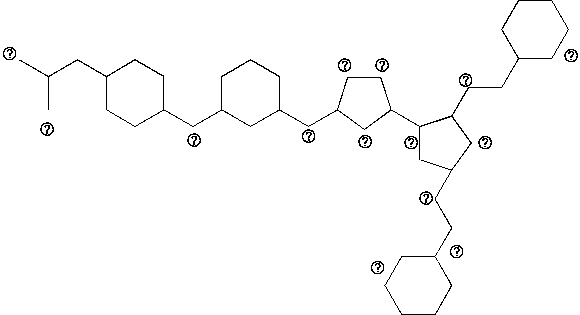 | 2-{4-[3-({5-[3,5-bis(phenylmethoxy)phenyl]- 1,3,4-thiadiazol-2-yl}amino)phenoxy]phenyl}acetic acid |
| 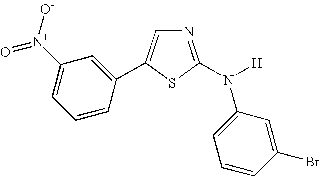 | (3-bromophen yl)[5-(3-nitrophenyl)(1,3-thiazol-2- yl)]amine |
| 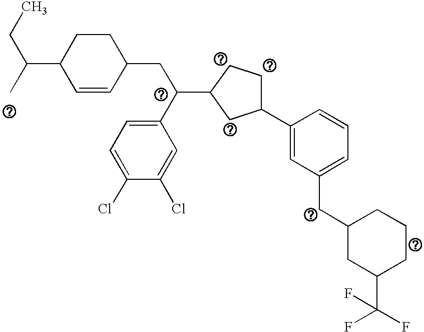 | methyl 4-{[(3,4-dichlorophenyl)(5-{3-[3-(tri- fluoromethyl)phenoxy]phenyl}(1,3,4-thiadiazol-2-yl))amino]methyl}benzoate |
| 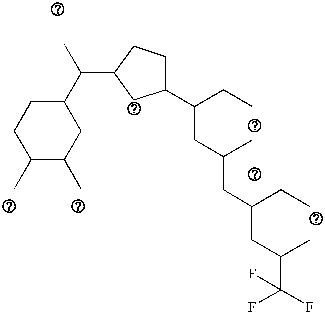 | 4-{[(3,4-dichlorophenyl)(5-{3-[3-(trifluoro- methyl)phenoxy]phenyl}(1,3,4-thiadiazol-2-yl))amino]methyl}benzoic acid |
| 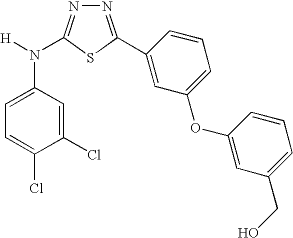 | (3,4-dichlorophenyl)(5-{3-[3-(oxymethyl)phenoxy]- phenyl}(1,3,4-thiadiazol-2-yl))amine |
| 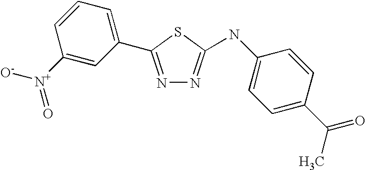 | 1-(4-{[5-(3-nitrophenyl)-1,3,4-thiadiazol-2-yl]- amino}phenyl)ethan-1-one |
| 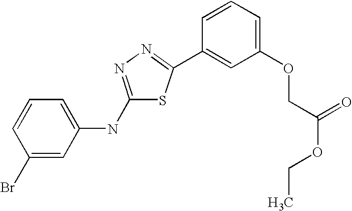 | ethyl 2-(3-{5-[(3-bromophenyl)amino]-1,3,4-thia- diazol-2-yl}phenoxy)acetate |
| 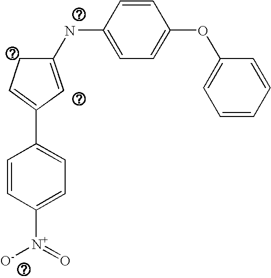 | [4-(4-nitrophenyl)(1,3-thiazol-2-yl)](4-phenoxy- phenyl)amine |
| 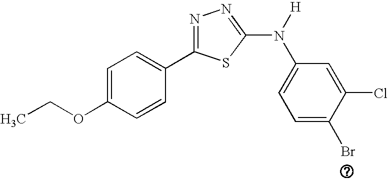 | (4-bromo-3-chlorophenyl)[5-(4-ethoxyphenyl)(1,3,4-thia- diazol-2-yl)]amine |
| 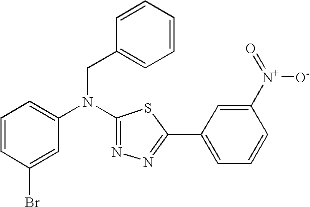 | (3-bromophenyl)[5-(3-nitrophenyl)(1,3,4-thiadiazol- 2-yl)]benzylamine |
| 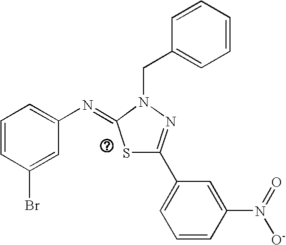 | 2-[aza-(3-bromophenyl)methylene]-5-(3-nitro- phenyl)-3-benzyl-1,3,4-thiadiazoline |
| 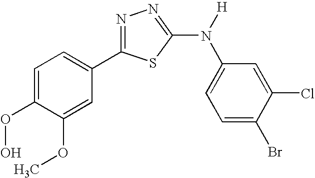 | [5-(3,4-dimethoxyphenyl)(1,3,4-thiadiazol-2- yl)](4-bromo-3-chlorophenyl)amine |
| 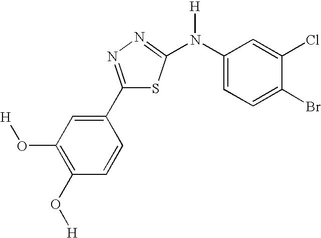 | 4-{5-[(4-bromo-3-chlorophenyl)amino]-1,3,4- thiadiazol-2-yl}benzene-1,2-diol |
| 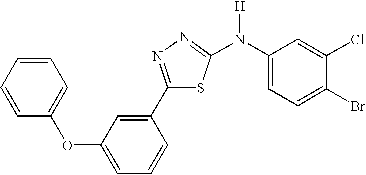 | (4-bromo-3-chlorophenyl)[5-(3-phenoxyphenyl)(1,3,4- thiadiazol-2-yl)]amine |
| 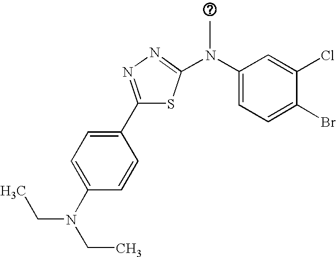 | (4-{5-[(4-bromo-3-chlorophenyl)amino](1,3,4- thiadiazol-2-yl)}phenyl)diethylamine |
| 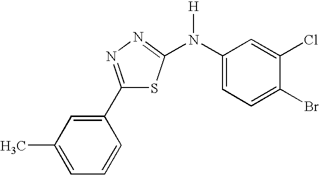 | (4-bromo-3-chlorophenyl)[5-(3-0methylphenyl)(1,3,4- thiadiazol-2-yl)]amine |
| 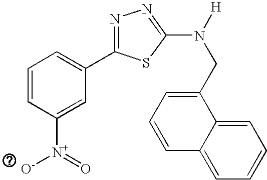 | (naphthylmethyl)[5-(3-nitrophenyl)(1,3,4-thiadiazol- 2-yl)]amine |
| 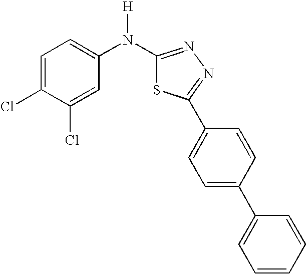 | (3,4-dichlorophenyl)[5-(4-phenylphenyl)(1,3,4-thia- diazol-2-yl)]amine |
| 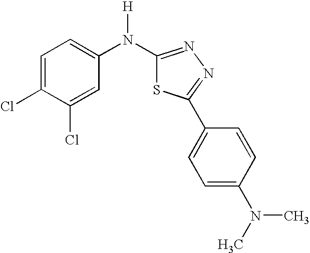 | (4-{5-[(3,4-dichlorophenyl)amino](1,3,4-thia- diazol-2-yl)}phenyl)dimethyamine |
| 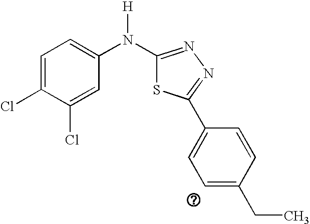 | (3,4-dichlorophenyl)[5-(4-methylthiophenyl)(1,3,4-thia- diazol-2-yl)]amine |
| 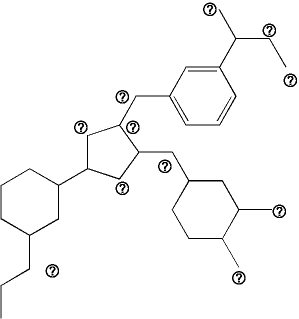 | methyl-3-({2-[aza(3,4-dichlorophenyl)methylene]- -(3-ethoxyphenyl)-1,m3,4-thiadiazol-3-yl}methyl)benzoate |
| 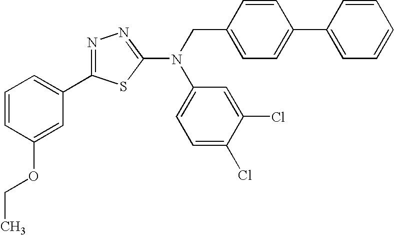 | (3,4-dichlorophenyl)[5-(3-ethoxyphenyl)(1,3,4-thia- diazol-2-yl)][(4-phenylphenyl)methyl]amineethyl)benzoate |
| 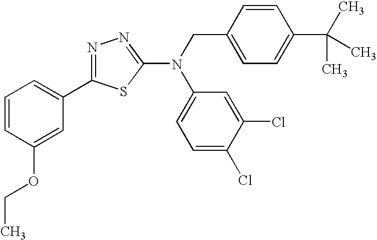 | (3,4-dichlorophenyl){[4-(tert-butyl)phenyl]- methyl}[5-(3-ethoxyphenyl)-1,3,4-thiadiazol-2-yl)]amine |
| 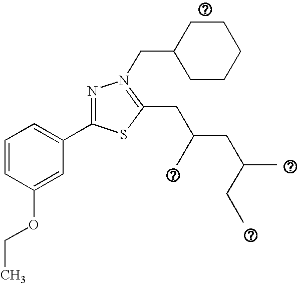 | 1-({2-[aza(3,4-dichlorophenyl)methylene]-5-(3- ethoxyphenyl)(1,3,4-thiadiazolin-3-yl)}methyl)-3-methoxybenzene |
| 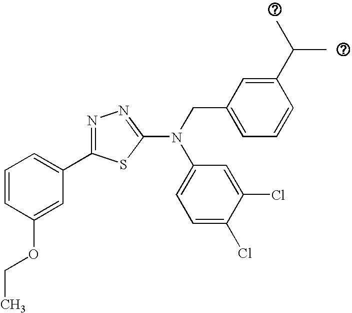 | (3,4-dichlorophenyl)[5-(3-ethoxyphenyl)(1,3,4-thia- diazol-2-yl)][(3-nitrophenyl)methyl]amine |
| 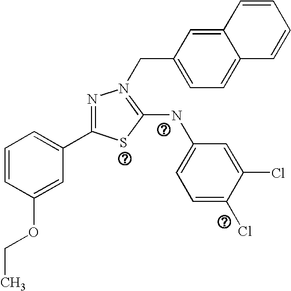 | 3-{2-[aza(3,4-dichlorophenyl)methylene]-3-(2- naphthylmethyl)(1,3,4-thiadiazolin-5-yl)}-1-ethoxybenzene |
| 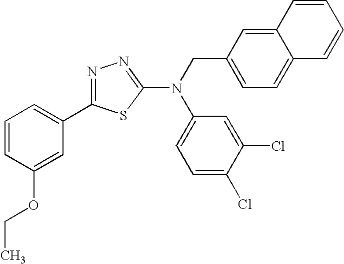 | (3,4-dichlorophenyl)[5-(3-ethoxyphenyl)(1,3,4-thia- diazol-2-yl)](2-naphthylmethyl)amine |
| 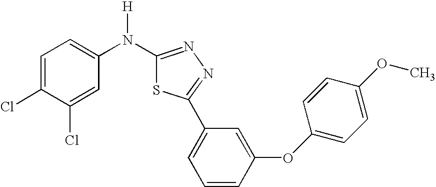 | (3,4-dichlorophenyl){5-[3-(4-methoxyphenoxy)- phenyl](1,3,4-thiadiazol-2-yl)}-amine |
| 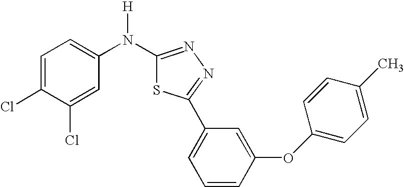 | (3,4-dichlorophenyl){5-[3-(4-methylphenoxy)- phenyl](1,3,4-thiadiazol-2-yl)}amine |
| 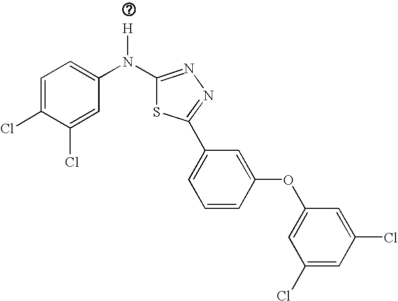 | (3,4-dichlorophenyl){5-[3-(3,5-dichlorophenoxy)- phenyl](1,3,4-thiadiazol-2-yl)}amine |
| 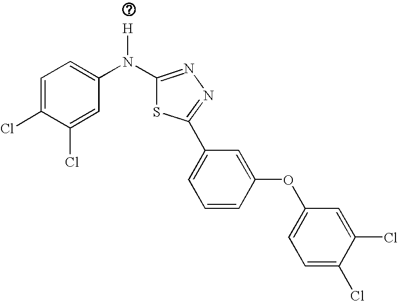 | (3,4-dichlorophenyl){5-[3-(3,4-dichlorophenoxy)- phenyl](1,3,4-thiadiazol-2-yl)}amine |
| 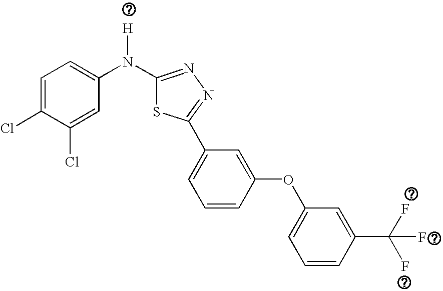 | (3,4-dichlorophenyl)(5-{3-[3-(trifluoromethyl)- phenoxy]phenyl}(1,3,4-thiadiazol-2-yl))amine |
| 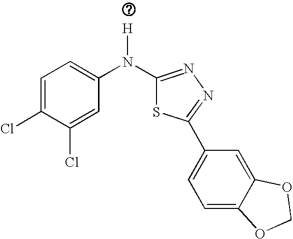 | (5-(2H-benzo[d]1,3-dioxolan-5-yl)(1,3,4-thia- diazol-2-yl))(3,4-dichlorophenyl)amine |
| 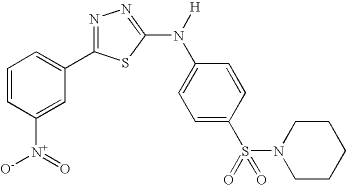 | [(4-{[5-(3-nitrophenyl)-1,3,4-thiadiazol-2- yl]amino}phenyl)sulfonyl]piperidine |
| 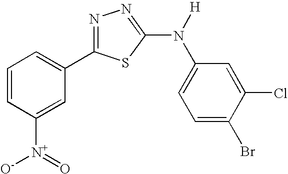 | (4-bromo-3-chlorophenyl)[5-(3-nitrophenyl)(1,3,4- thiadiazol-2-yl)]amine |
| 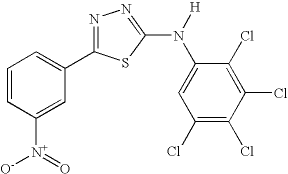 | [5-(3-nitrophenyl)(1,3,4-thiadiazol-2-yl)]- (2,3,4,5-tetrachlorophenyl)amine |
| 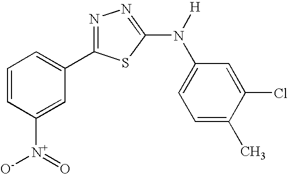 | (3-chloro-4-methylphenyl)[5-(3-nitrophenyl)- (1,3,4-thiadiazol-2-yl)]amine |
| 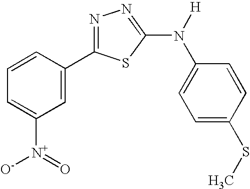 | (4-methylphenyl)[5-(3-nitrophenyl)(1,3,4-thia- diazol-2-yl)]amine |
| 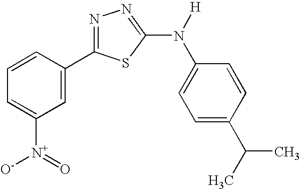 | [4-(methylethyl)phenyl][5-(3-nitrophenyl)- (1,3,4-thiadiazol-2-yl)]amine |
| 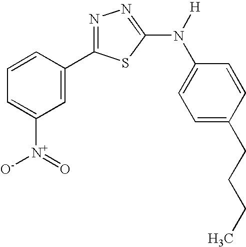 | (4-butylphenyl)[5-(3-nitrophenyl)(1,3,4-thia- diazol-2-yl)]amine |
| 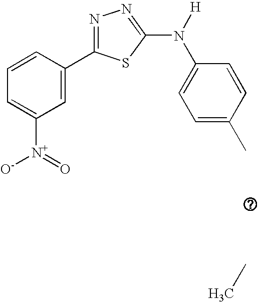 | (4-decylphenyl)[5-(3-nitrophenyl)(1,3,4-thia- diazol-2-yl)]amine |
| 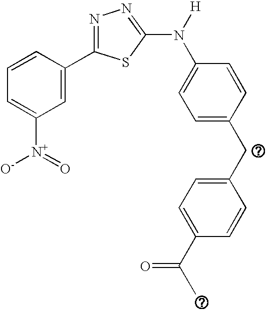 | [5-(3-nitrophenyl)(1,3,4-thiadiazol-2-yl)]- [4-(4-nitrophenoxy)phenyl]amine |
| 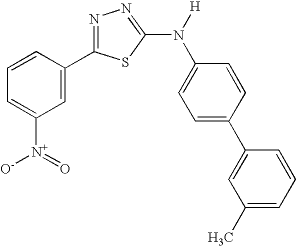 | (3-methylphenyl)[5-(3-nitrophenyl)(1,3,4-thia- diazol-2-yl)]amine |
| 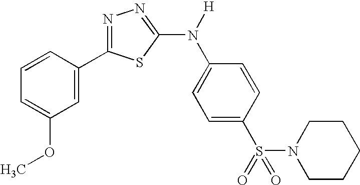 | [(4-{[5-(3-methoxyphenyl)-1,3,4-thiadia- zol-2-yl]amino}phenyl)sulfonyl]piperidine |
| 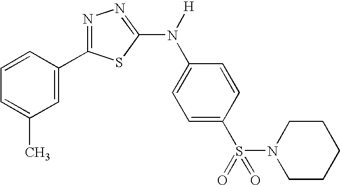 | [(4-{[5-(3-methylphenyl)-1,3,4-thiadia- zol-2-yl]amino}phenyl)sulfonyl]piperidine |
| 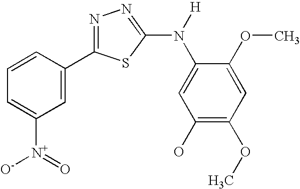 | (5-chloro-2,4-dimethoxyphenyl)[5-(3-nitro- phenyl)(1,3,4-thiadiazol-2-yl)]amine |
| 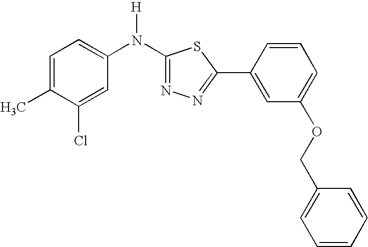 | (3-chloro-4-methylphenyl){5-[3-(phenylmeth- oxy)phenyl](1,3,4-thiadiazol-2-yl)}amine |
| 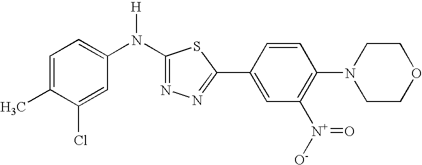 | (3-chloro-4-methylphenyl)[5-(4-morpholin-4-yl- 3-nitrophenyl)(1,3,4-thiadiazol-2-yl)]amine |
| 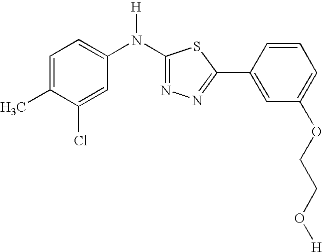 | 2-(3-{5-[(3-chloro-4-methylphenyl)amino]- 1,3,4-thiadiazol-2-yl}phenoxy)ethan-1-ol |
| 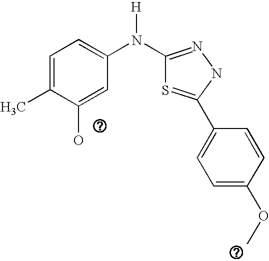 | (3-chloro-4-methylphenyl){5-[4-(trifluoro- methylthio)phenyl](1,3,4-thiadiazol-2-yl)}amine |
| 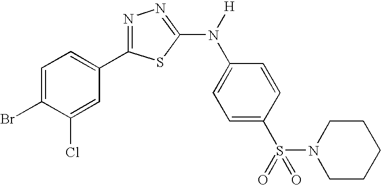 | [(4-{[5-(4-bromo-3-chlorophenyl)-1,3,4-thia- diazol-2-yl]amino}phenyl)sulfonyl]piperidine |
| 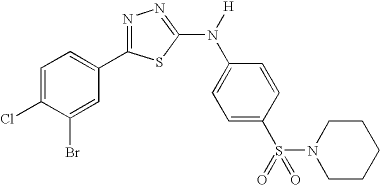 | [(4-{[5-(3-bromo-4-chlorophenyl)-1,3,4-thia- diazol-2-yl]amino}phenyl)sulfonyl]piperidine |
| 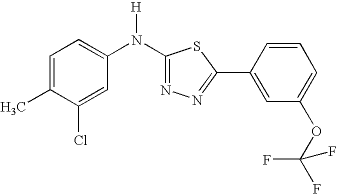 | (3-chloro-4-methylphenyl){5-[3-(trifluorometh- oxy)phenyl](1,3,4-thiadiazol-2-yl)}amine |
| 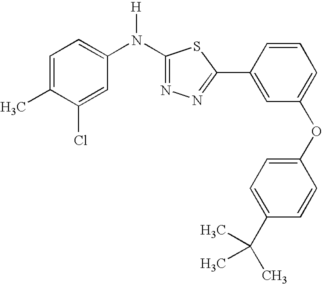 | (5-{3-[4-(tert-butyl)phenoxy]phenyl}(1,3,4- thiadiazol-2-yl))(3-chloro-4-methylphenyl)amine |
| 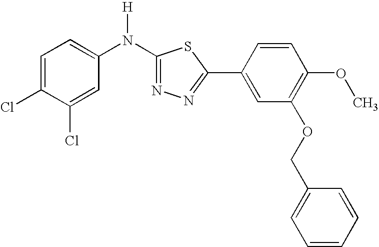 | (3,4-dichlorophenyl){5-[4-methoxy-3-(phenylmeth- oxy)phenyl](1,3,4-thiadiazol-2-yl)}amine |
| 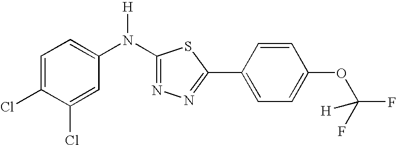 | (3,4-dichlorophenyl){5-[4-(trifluoromethoxy)- phenyl](1,3,4-thiadiazol-2-yl)}amine |
| 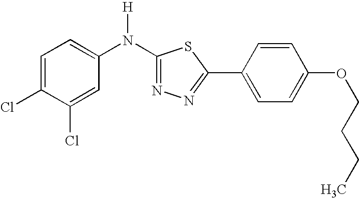 | (3,4-dichlorophenyl)[5-(4-butoxyphenyl)(1,3,4-thia- diazol-2-yl)]amine |
| 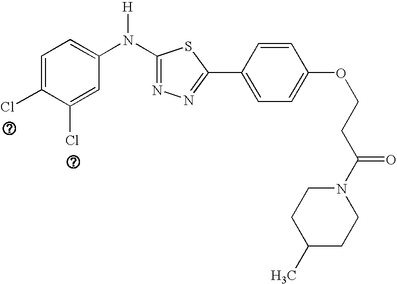 | 2-(3-{5-[(3,4-dichlorophenyl)amino](1,3,4-thia- diazol-2-yl)}phenoxy)-1-(4-methylpiperidyl)ethan-1-one |
| 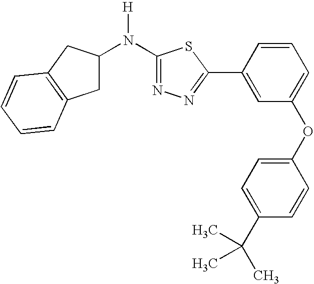 | (5-{3-[4-(tert-butyl)phenoxy]phenyl}(1,3,4- thiadiazol-2-yl))indan-2-ylamine |
| 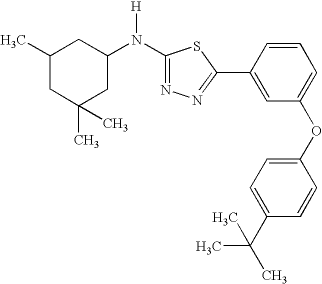 | (5-{3-[4-(tert-butyl)phenoxy]phenyl}(1,3,4- thiadiazol-2-yl))(3,3,5-trimethylcyclohexyl)amine |
| 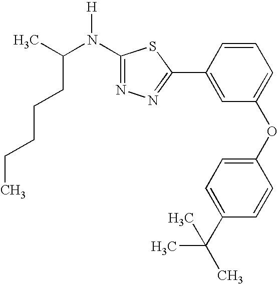 | (5-{3-[4-(tert-butyl)phenoxy]phenyl}(1,3,4- thiadiazol-2-yl))(methylhexyl)amine |
| 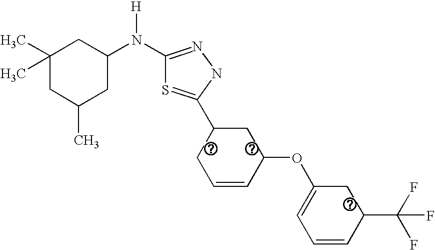 | (5-{3-[3-(trifluoromethyl)phenoxy]phenyl}- (1,3,4-thiadiazol-2-yl))(3,3,5-trimethylcyclohexyl)amine |
| 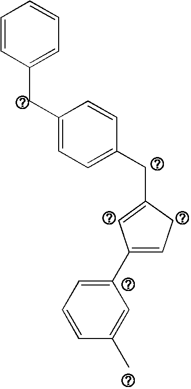 | (4-bromo-3-chlorophenyl)[5-(3-phenoxyphenyl)(1,3,4- thiadiazol-2-yl)]amine |
| 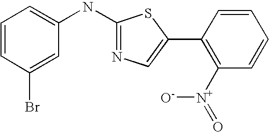 | (3-bromophenyl)[5-(2-nitrophenyl)(1,3-thiazol-2- yl)]amine |
| 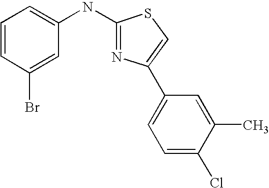 | (3-bromophenyl)[4-(4-chloro-3-meethylphenyl)(1,3- thiazol-2-yl)]amine |
| 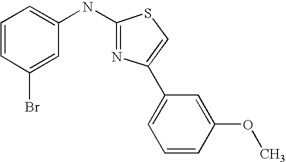 | (3-bromophenyl)[4-(3-methoxyphenyl)(1,3-thiazol- 2-yl)]amine |
| 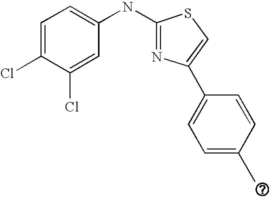 | (4-{2-[(3,4-dichlorophenyl)amino](1,3-thia- zol-4-yl)}phenyl)diethylamine |
| 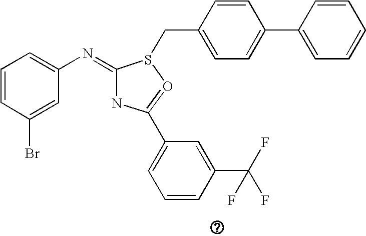 | N-{2-aza-2-(3-bromophenyl)-1-[(4-phenylphenyl)- methylthio]vinyl}[3-(trifluoromethyl)phenyl]carboxamide |
| 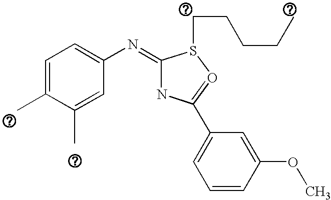 | (2Z)-2-aza-3-[(3,4-dichlorophenyl)amino]-1-(3- methoxyphenyl)-3-{[3-(trifluoromethylthio)phenyl]methylthio}prop-2-en-1-one |
| 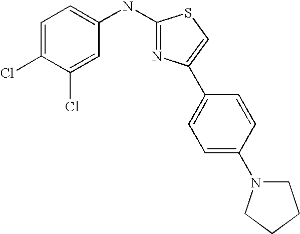 | (3,4-dichlorophenyl)[4-(4-pyrrolidinylphenyl)(1,3- thiazol-2-yl)]amine |
| 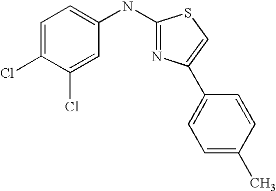 | 4-{2-[(3,4-dichlorophenyl)amino]-1,3-thiazol- 4-yl}benzenecarbonitrile |
| 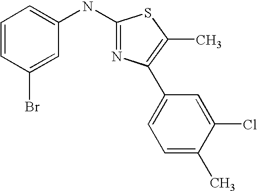 | (3-bromophenyl)[4-(3-chloro-4-methylphenyl)-5- methyl(1,3-thiazol-2-yl)]amine |
| 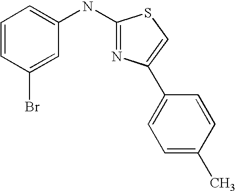 | (3-bromophenyl)[4-(4-methylphenyl)(1,3-thiazol- 2-yl)]amine |
| 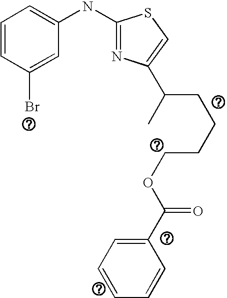 | 3-{2-[(3-bromophenyl)amino]-1,3-thiazol-4- yl}phenyl benzoate |
| 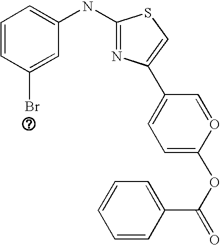 | 4-{2-[(3-bromophenyl)amino]-1,3-thiazol-4- yl}phenyl benzoate |
| 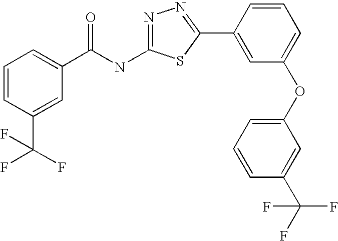 | [3-(trifluoromethyl)phenyl]-N-(5-{3-[3- (trifluoromethyl)phenoxy]phenyl}(1,3,4-thiadiazol-2-yl))carboxamide |
| 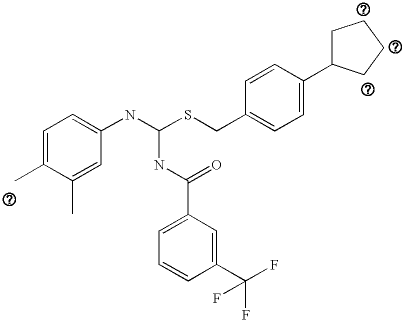 | N-{2-aza-2-(3,4-dichlorophenyl)-1-[(4-(1,2,3- thiadiazol-4-yl)phenyl)methylthio]vinyl}[3-(trifluoromethyl)phenyl]carboxamide |
| 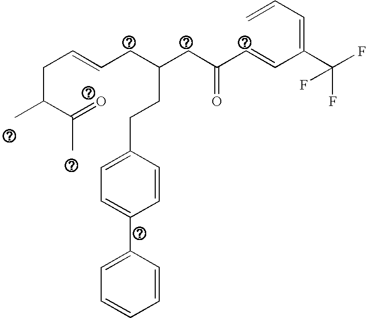 | N-{2-aza-2-(3,4-dichlorophenyl)-1-[(4-phenyl- phenyl)methylthio]vinyl}[3-(trifluoromethyl)phenyl]carboxamide |
| 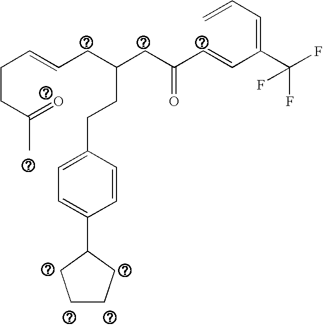 | N-{2-aza-2-(3-bromophenyl)-1-[(4-(1,2,3-thia- diazol-4-yl)phenyl)methylthio]vinyl}[3-(trifluoromethyl)phenyl}carboxamide |
| 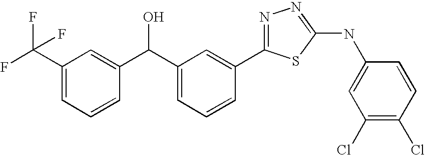 | (3-{5-[(3,4-dichlorophenyl)amino](1,3,4-thia- diazol-2-yl)}phenyl)[3-(trifluoromethyl)phenyl]methan-1-ol |
| 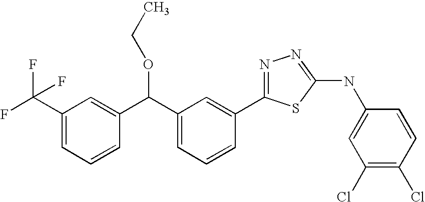 | (3,4-dichlorophenyl)[5-(3-(ethoxy[3-(trifluoro- methyl)phenyl]methyl}phenyl)(1,3,4-thiadiazol-2-yl)]amine |
| 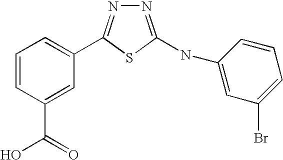 | 3-{5-[(3-bromophenyl)amino]-1,3,4-thiadiazol- -yl}benzoic acid |
| 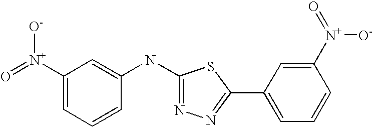 | (3-nitrophenyl)[5-(3-nitrophenyl)(1,3,4-thiadiazol- 2-yl)]amine |
| 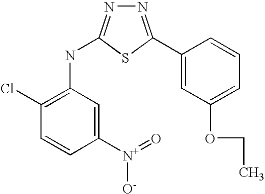 | (2-chloro-5-nitrophenyl)[5-(3-ethoxyphenyl)(1,3,4- thiadiazol-2-yl)]amine |
| 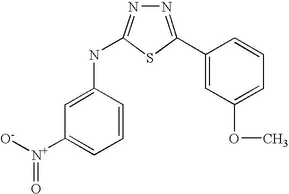 | [5-(3-methoxyphenyl)(1,3,4-thiadiazol-2-yl)](3- nitrophenyl)amine |
| 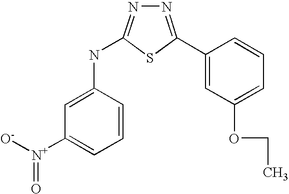 | [5-(3-ethoxyphenyl)(1,3,4-thiadiazol-2-yl)](3- nitrophenyl)amine |
| 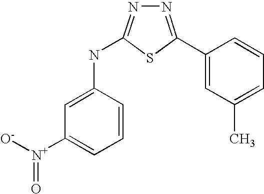 | [5-(3-methylphenyl)(1,3,4-thiadiazol-2-yl)](3- nitrophenyl)amine |
| 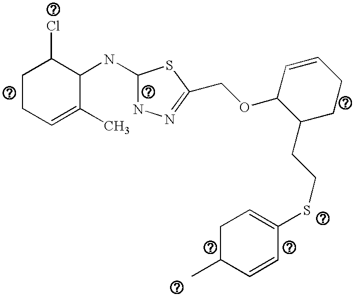 | Deleted Entry |
| 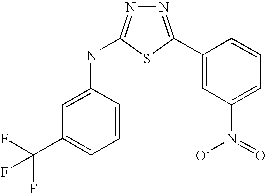 | [5-(3-nitrophenyl)(1,3,4-thiadiazol-2-yl)][3- (trifluoromethyl)phenyl]amine |
| 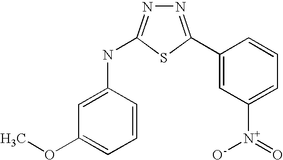 | (3-methoxyphenyl)[5-(3-nitrophenyl)(1,3,4-thia- diazol-2-yl)]amine |
| 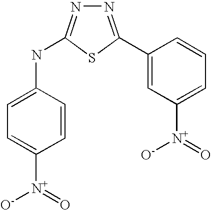 | (4-nitrophenyl)[5-(3-nitrophenyl)(1,3,4-thia- diazol-2-yl)]amine |
| 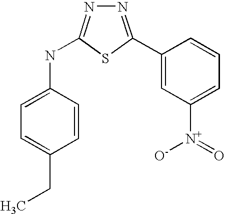 | (4-ethylphenyl)[5-(3-nitrophenyl)(1,3,4-thia- diazol-2-yl)]amine |
| 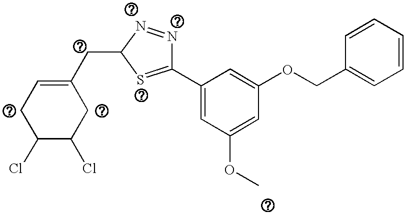 | (3,4-dichlorophenyl){5-[3,5-bis(phenyl- methoxy)phenyl](1,3,4-thiadiazol-2-yl)}-amine |
| 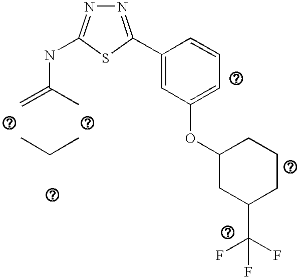 | [3-(phenylmethoxy)phenyl](5-{3-[3-(tri- fluoromethyl)phenoxy]phenyl}(1,3,4-thidiazol-2-yl))amine |
| 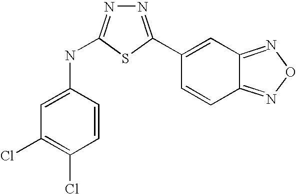 | (5-benzo[c]1,2,5-oxadiazol-5-yl(1,3,4-thia- diazol-2-yl))(3,4-dichlorophenyl)amine |
| 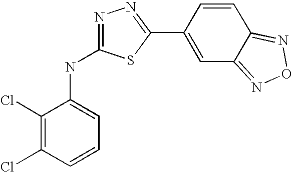 | (5-benzo[3,4-c]1,2,5-oxadiazol-5-yl(1,3,4-thia- diazol-2-yl))(2,3-dichlorophenyl)amine |
| 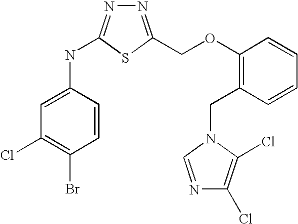 | [5-({2-[(4,5-dichloroimidazolyl)methyl]phen- oxy}methyl)(1,3,4-thiadiazol-2-yl)](4-bromo-3-chlorophenyl)amine |
| 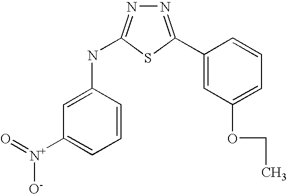 | Deleted Entry |
| 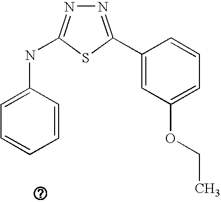 | (4-{[5-(3-ethoxyphenyl)(1,3,4-thiadiazol-2 -yl)]amino}phenyl)(phenylsulfonyl)amine |
| 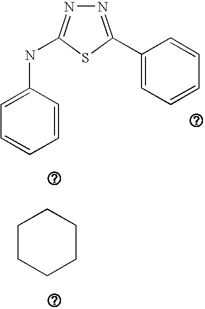 | [(4-methylphenyl)sulfonyl](4-{[5-(3- nitrophenyl)(1,3,4-thiadiazol-2-yl)amino}phenyl)amine |
| 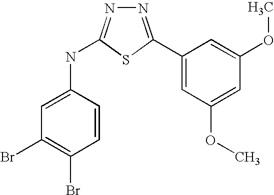 | (3,4-dibromophenyl)[5-(3,5-dimethoxyphenyl)- (1,3,4-thiadiazol-2-yl)]amine |
| 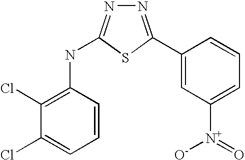 | (2,3-dichlorophenyl)[5-(3-nitrophenyl)(1,3,4- thiadiazol-2-yl)]amine |
| 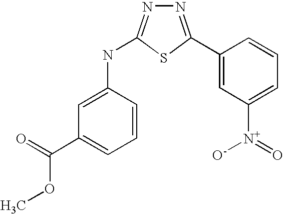 | methyl 3-{[5-(3-nitrophenyl)-1,3,4-thia- diazol-2-yl]amino}benzoate |
| 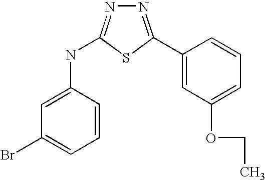 | (3-bromophenyl)[5-(3-ethoxyphenyl)(1,3,4-thia- diazol-2-yl)]amine |
| 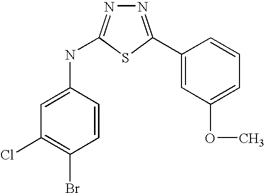 | (4-bromo-3-chlorophenyl)[5-(3-methoxyphenyl)- (1,3,4-thiadiazol-2-yl)]amine |
| 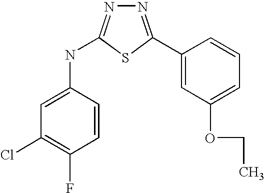 | (3-chloro-4-fluorophenyl)[5-(3-ethoxyphenyl)- (1,3,4-thiadiazol-2-yl)]amine |
| 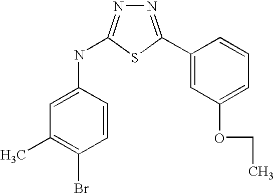 | (4-bromo-3-methylphenyl)[5-(3-ethoxyphenyl)- (1,3,4-thiadiazol-2-yl)]amine |
| 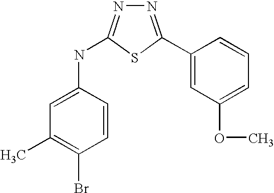 | (4-bromo-3-methylphenyl)[5-(3-methoxyphenyl)- (1,3,4-thiadiazol-2-yl)]amine |
| 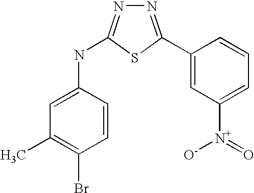 | (4-bromo-3-methylphenyl)[5-(3-nitrophenyl)- (1,3,4-thiadiazol-2-yl)]amine |
| 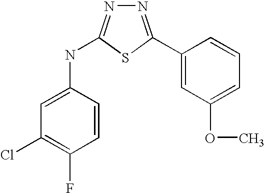 | (3-chloro-4-fluorophenyl)[5-(3-methoxyphenyl)- (1,3,4-thiadiazol-2-yl)]amine |
| 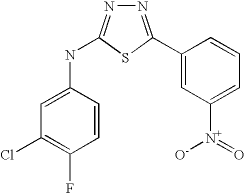 | (3-chloro-4-fluorophenyl)[5-(3-nitrophenyl)- (1,3,4-thiadiazol-2-yl)]amine |
| 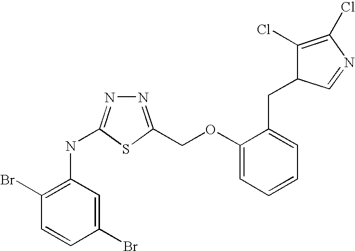 | Deleted Entry |
| 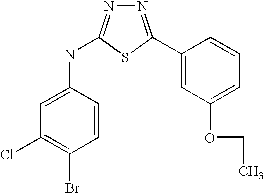 | (4-bromo-3-chlorophenyl)[5-(3-ethoxyphenyl)- (1,3,4-thiadiazol-2-yl)]amine |
| 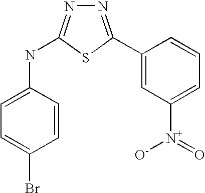 | (4-bromophenyl)[5-(3-nitrophenytl)(1,3,4-thia- diazol-2-yl)]amine |
| 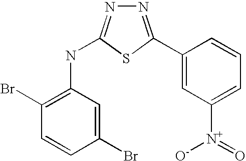 | (2,5-dibromophenyl)[5-(3-nitrophenyl)(1,3,4-thia- diazol-2-yl)]amine |
| 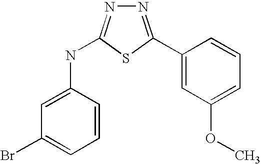 | (3-bromophenyl)[5-(3-methoxyphenyl)(1,3,4-thia- diazol-2-yl)]amine |
| 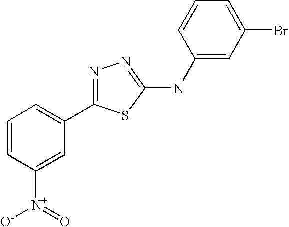 | (3-bromophenyl)[5-(3-nitrophenyl)(1,3,4-thia- diazol-2-yl)]amine |
| 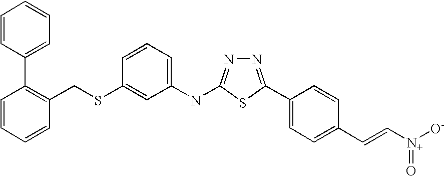 | ammonium 3-{4-[5-({3-[(2-phenylphenyl)- methylthio]phenyl}amino)(1,3,4-thiadiazol-2-yl)]phenyl}prop-2-enoate |
| 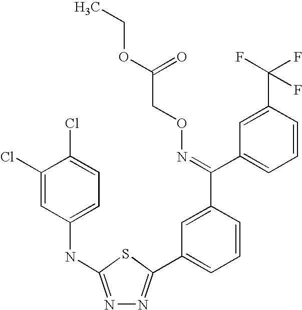 | ethyl 2-[(1Z)-1-aza-2-(3-{5-[(3,4-dichloro- phenyl)amino](1,3,4-thiadiazol-2-yl)}phenyl)-2-[3-(trifluoromethyl)phenyl]vinyloxy]acetate |
| 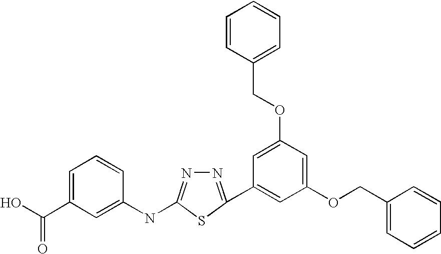 | 3-({5-[3,5-bis(phenylmethoxy)phenyl]-1,3,4- thiadiazol-2-yl}amino)benzoic acid |
| 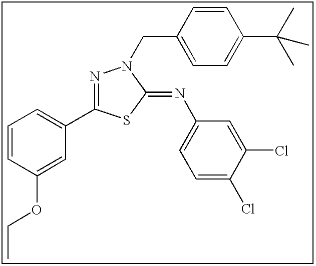 | (1-(2-[aza(3,4-dichlorophenyl)methylene]-3 -{[4-(tert-butyl)phenyl]methyl}(1,3,4-thiadiazolin-5-yl))-3-ethoxybenzene) |
| Structure | Name | Compound # |
|---|---|---|
| 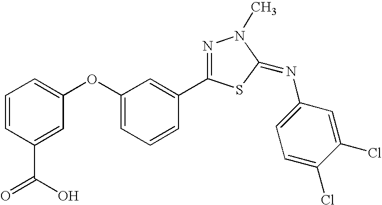 | 3-{3-[5-(3,4-Dichloro- phenylimino)-4-methyl-4,5- dihydro-[1,3,4]thiadiazol-2- yl]-phenoxy}-benzoic acid | 164 |
| 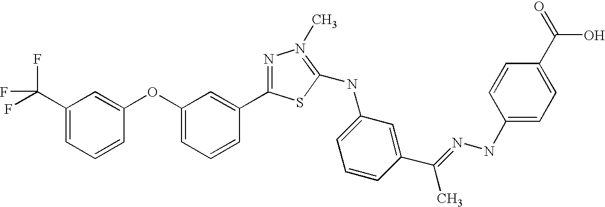 | 4-{N′-[1-(3-{5-[3-(3- Trifluoromethyl-phenoxy)- phenyl]-[1,3,4]thiadiazol-2- ylamino}-phenyl)-ethylidene]- hydrazino}-benzoic acid | 165 |
| 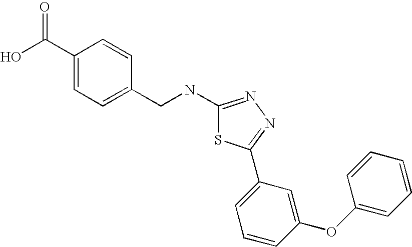 | 4-{[5-(3-Phenoxy-phenyl)- [1,3,4]thiadiazol-2-ylamino]- methyl}-benzoic acid | 166 |
| 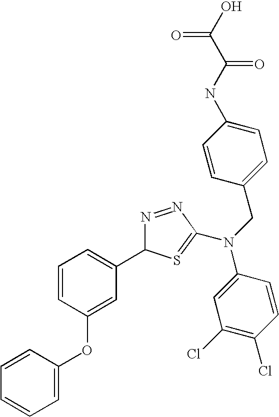 | 3,4[thiadiazol-2-yl]-amino}- methyl)-phenyl]-oxalamicacid | 167 |
| 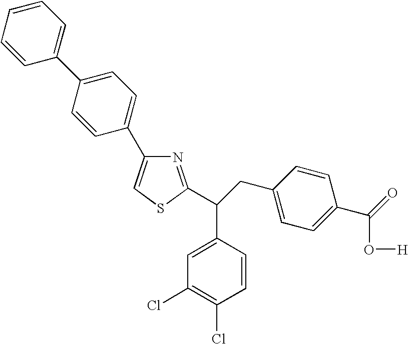 | 4-{[(4-Biphenyl-4-yl-thiazol- 2-yl)-(3,4-dichloro-phenyl)- amino]-methyl}-benzoic acid | 168 |
| 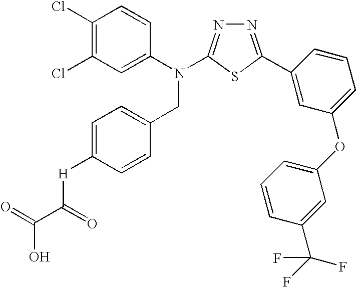 | N-{4-[((3,4-Dichloro-phenyl)- {5-[3-(3-trifluoromethyl- phenoxy)-phenyl]- [1,3,4]thiadiazol-2yl}-amino)- methyl]-phenyl}-oxalamic acid | 169 |
| 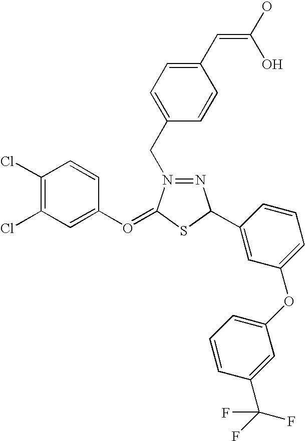 | (4-{2-(3,4-Dichloro- phenylimino)-5-[3-(3- trifluoromethyl-phenoxy)- phenyl]-[1,3,4]thiadiazol-3-yl- methyl}-phenyl)- acetic acid | 170 |
| 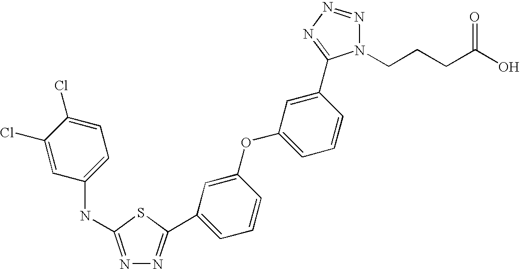 | 4-[5-(3-{3-[5-(3,4-Dichloro- phenylamino)- [1,3,4]thiadiazol-2-yl]- phenoxy}-phenyl)-tetrazol-1- yl]-butyric acid | 171 |
| 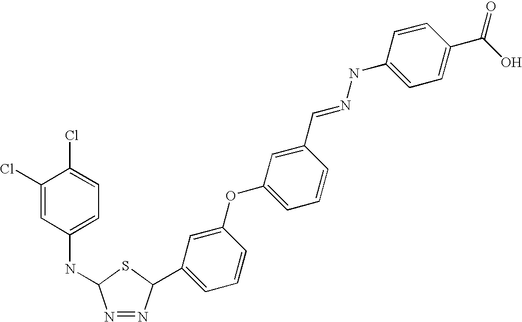 | 4-[N′-(3-{3-[5-(3,4-Dichloro- phenylamino)- [1,3,4]thiadiazol-2-yl]- phenoxy}-benzylidene)- hydrazino]-benzoic acid | 172 |
| 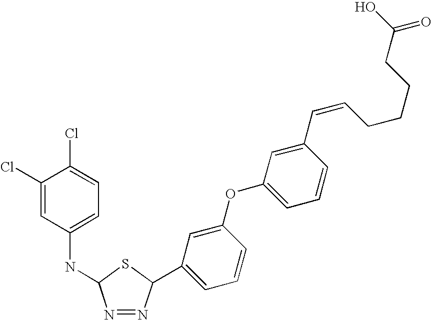 | 7-(3-{3-[5-(3,4-Dichloro- phenylamino)- [1,3,4]thiadiazol- 2yl]-phenoxy}-phenyl)-hept- 6-enoic acid | 173 |
| 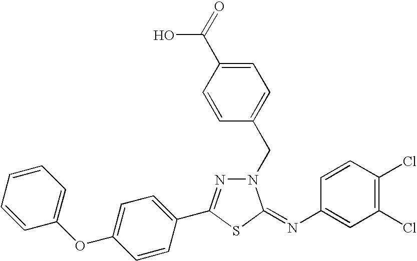 | 4-[2-(3,4-Dichloro- phenylimino)-5-(4-phenoxy- phenyl)-[1,3,4]thiadiazol-3- ylmethyl]-benzoic acid. | 174 |
| 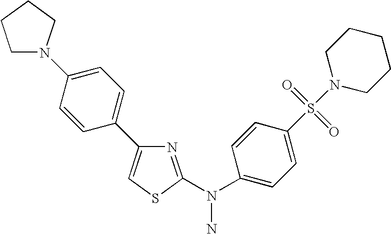 | [4-(Piperidine-1-sulfonyl)- phenyl]-[4-(4-pyrrolidin-1-yl- phenyl)-thiazol-2-yl]-amine | 175 |
| 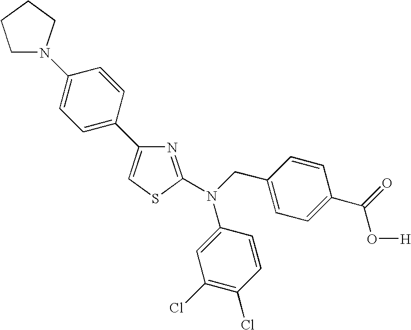 | 4-({(3,4-Dichloro-phenyl)- [4-(4-pyrrolidin-1-yl-phenyl)- thiazol-2-yl]-amino}-methyl)- benzoic acid | 176 |
| 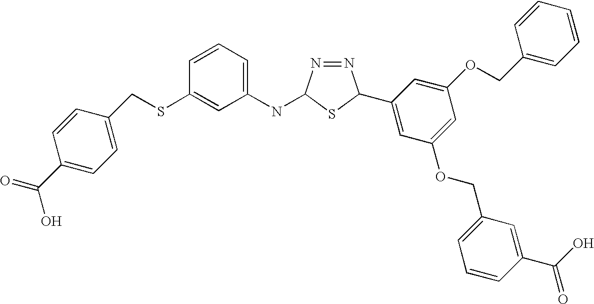 | 4-{3-[5-((3-(3- Hydroxycarbonylbenzyl)-5- Benzyloxy-phenyl)- [1,3,4]thiadiazol-2-ylamino]- phenylsulfanylmethyl}- benzoic acid | 177 |
| 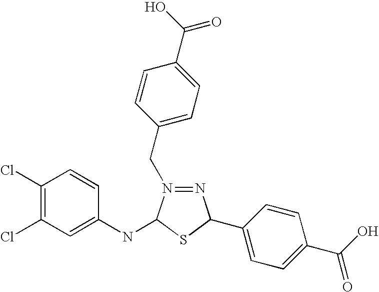 | 4-[4-(4-hydroxycarbonyl- benzyl)-5-(3,4-dichloro- phenylimino)-4,5-dihydro- [1,3,4]thiadiazol-2-yl]benzoic acid | 178 |
| 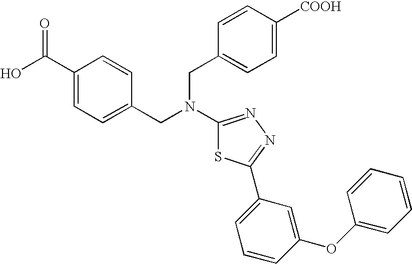 | 4-({(4-Hydroxycarbonyl- benzyl-[5-(3-phenoxy- phenyl)-[1,3,4]thiadiazol- 2-yl]-amino}-methyl)- benzoic acid | 179 |
| 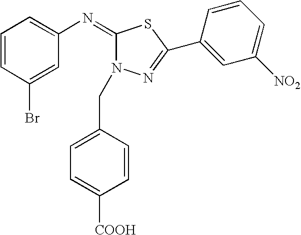 | 4-[2-(3-Bromo-phenylimino)- 5-(3-nitro-phenyl)- [1,3,4]thiadiazol-3-ylmethyl]- benzoic acid | 180 |
| 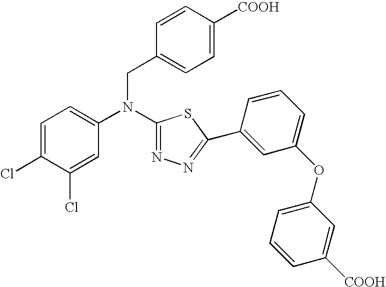 | 3-(3-{5-[(4-Hydroxycarbonyl- benzyl)-(3,4-dichloro-phenyl)- amino]-[1,3,4]thiadiazol-2- yl}-phenoxy)-benzoic acid | 181 |
| 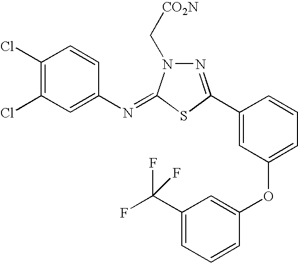 | {2-(3,4-Dichloro- phenylimino)-5-[3-(3- trifluoromethyl-phenoxy)- phenyl]-[1,3,4]thiadiazol-3- yl}-acetic acid | 182 |
| 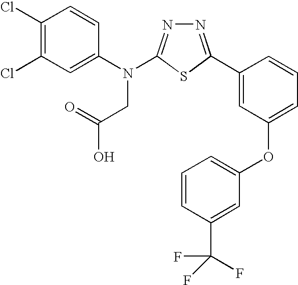 | ((3,4-Dichloro-phenyl)-{5- [3-(3-tifluoromethyl- phenoxy)-phenyl]- [1,3,4]thiadiazol-2-yl}- amino)-acetic acid | 183 |
| 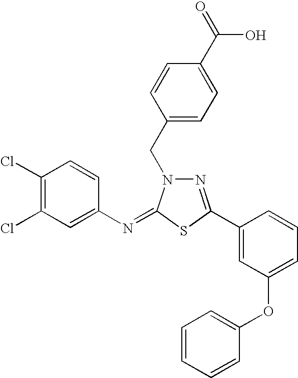 | 4-[2-(3,4-Dichloro- phenylimino)-5-(3-phenoxy- phenyl)-[1,3,4]thiadiazol-3- ylmethyl]-benzoic acid | 184 |
| 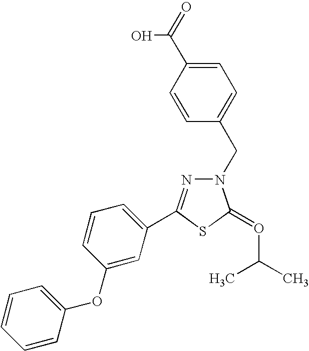 | 4-[2-Isopropylimino-5-(3- phenoxy-phenyl)- [1,3,4]thiadiazol-3-ylmethyl]- benzoic acid | 185 |
| 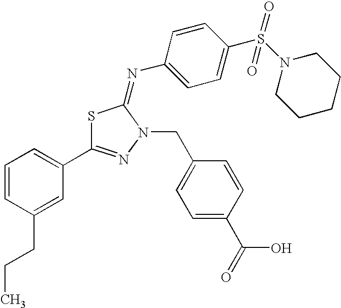 | 4-{5-(3-Ethoxy-phenyl)-2-[4- (piperidine-1-sulfonyl)- phenylimino]- [1,3,4]thiadiazol-3-ylmethyl}- benzoic acid | 186 |
| 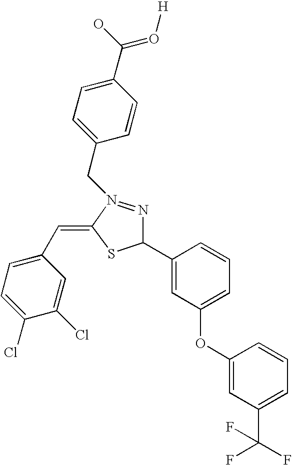 | 4-{2-(3,4-Dichloro- phenylimino)-5-[3{3- trifluoromethyl-phenoxy)- phenyl]-[1,3,4]thiadiaziol-3- ylmethyl}-benzoic acid | 187 |
| 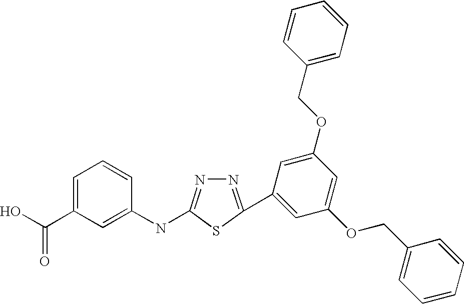 | 3-[5-(3,5-Bis-benzyloxy- phenyl)-[1,3,4]thiadiazol-2- ylamino]-benzoic acid | 188 |
| 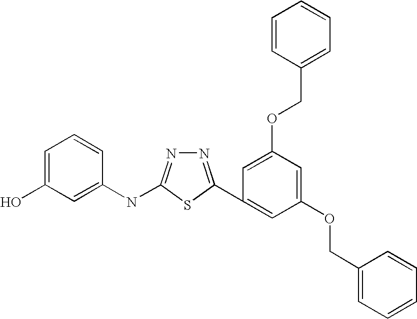 | 3-[5-(3,5-Bis-benzyloxy- phenyl)-[1,3,4]thiadiazol-2- ylamino]-phenol | 189 |
| 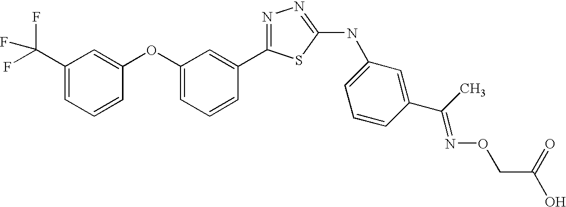 | [1-(3-{5-[3-(3- Trifluoromethyl-phenoxy)- phenyl]-[1,3,4]thiadiazol-2- ylamino}-phenyl)- ethylideneaminooxy]acetic acid | 190 |
| 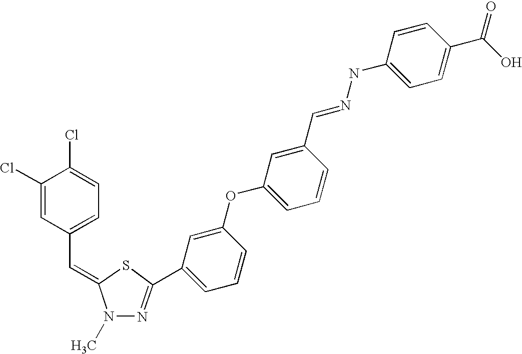 | 4-[N′-(3-{3-[5-(3,4-Dichloro- phenylimino-4-methyl-4,5- dihydro-[1,3,4]thiadiazol-2- yl]-phenoxy}-benzylidene)- hydrazino]-benzoic acid | 191 |
| 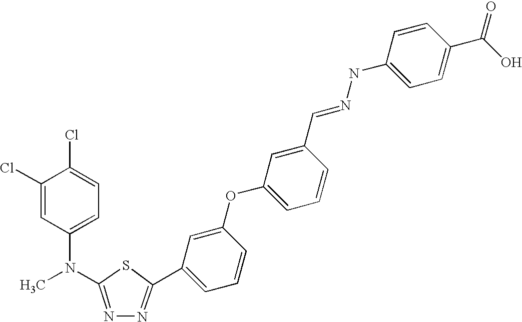 | 4-{N′[3-{5-[(3,4-Dichloro- phenyl)-methyl-amino)- [1,3,4]thiadiazol-2-yl}- phenoxy)-benzylidene[- hydrazino}-benzoic acid | 192 |
| 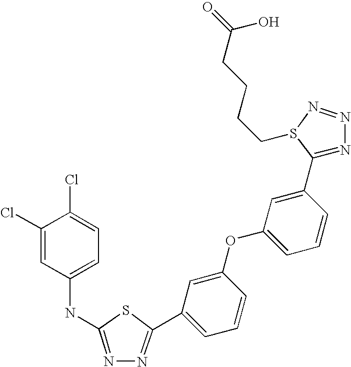 | 5-[5-(3-{3-[5-(3,4-Dichoro- phenylamino)- [1,3,4]thiadiazol-2-yl]- phenoxy}-phenyl)-tetrazol-1- yl]-pentanoic acid | 193 |
| 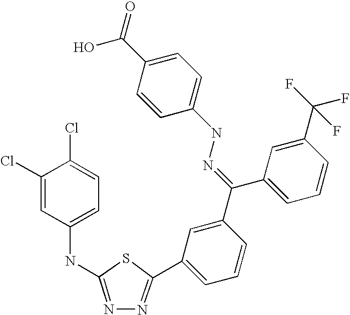 | 4-{N′-[{3-[5-(3,4-Dichloro- phenylamino)- [1,3,4]thiadiazol2-yl]- phenyl}-(3-trifluoromethyl- phenyl)-methylene]- hydrazino}-benzoic acid | 194 |
| 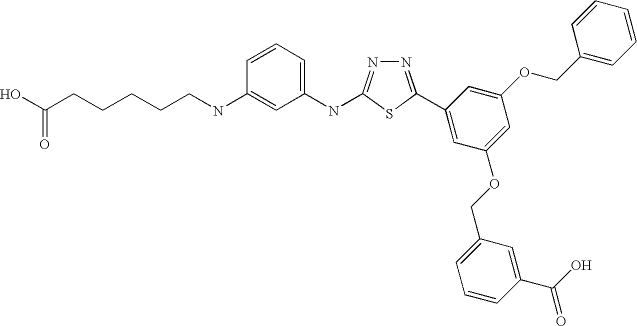 | 3-(3-Benzyloxy-5-{5-[3-(5- carboxy-pentylsulfanyl)- phenylamino]- [1,3,4]thiadiazol-2-yl}- phenoxymethyl)-benzoic acid | 195 |
| 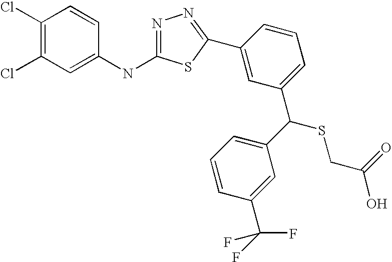 | {3-[5-(3,4-Dichloro- phenylamino)- [1,3,4]thiadiazol-2-yl]- phenyl}-(3-trifluoromethyl- phenyl)-methylsulfanyl]- acetic acid | 196 |
| 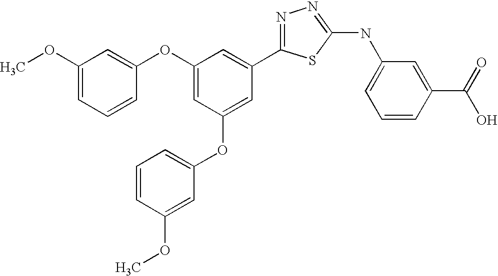 | 3-{5-[3,5-Bis-(3-methoxy- phenoxy)-phenyl]- [1,3,4]thiadiazol-2- ylamino}-benzoic acid | 197 |
| 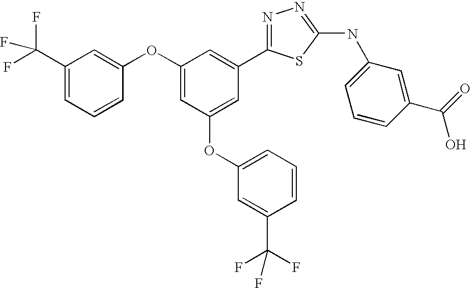 | 3-{5-[3,5-Bis-(3- trifluoromethyl-phenoxy)- phenyl]-[1,3,4]thiadiazol-2- ylamino}-benzoic acid | 198 |
| 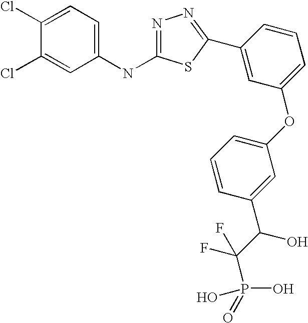 | [2-(3-{3-[5-(3,4-Dichloro- phenylamino)- [1,3,4]thiadiazol-2-yl]- phenoxy}-phenyl)-1,1- difluoro-2-hydroxy-ethyl]- phosphonic acid | 199 |
| 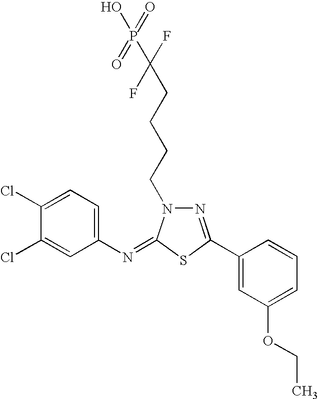 | {5-[2-(4-Bromo-3-chloro- phenylimino)-5-(3-ethoxy- phenyl)-[1,3,4]thiadiazol-3- yl]-1,1-difluoro-pentyl}- phosphonic acid | 200 |
| 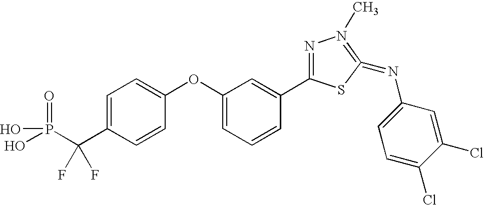 | [(4-{3-[5-(3,4-Dichloro- phenylimino)-4-methyl-4,5- dihydro-[1,3,4]thiadiazol-2- yl]-phenoxy}-phenyl)- difluoro-methyl]- phosphonic acid | 201 |
| 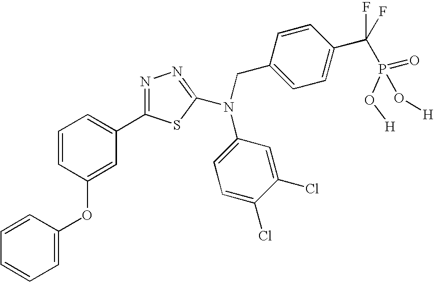 | {[4-({(3,4-Dichloro- phenyl)-[5-(3-phenoxy- phenyl)-[1,3,4]thiadiazol-2- yl]-amino}-methyl)-phenyl]- difluoro-methyl}- phosphonic acid | 202 |
| 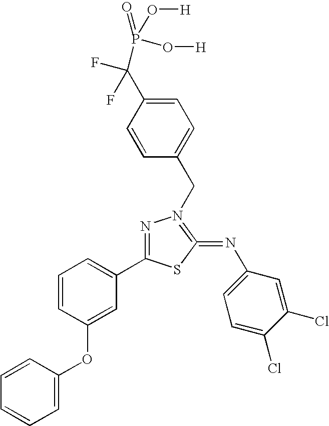 | ({4-[2-(3,4-Dichloro- phenylimino)-5-(3-phenoxy- phenyl)-[1,3,4]thiadiazol-3- ylmethyl]-phenyl}-difluoro- methyl)-phosphonic acid | 203 |
| 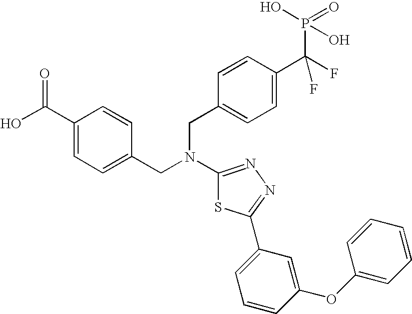 | 4-({[4-(Difluoro-phosphono- methyl)-benzyl]-[5-(3- phenoxy-phenyl)- [1,3,4]thiadiazol-2-yl]- amino}-methyl)-benzoic acid | 204 |
